(19) United States
(12) Patent Application Publication
(10) Pub. No.: US 2020/0330603 A1
(43) Pub. Date: Oct. 22, 2020

(54) PEPTIDES FOR RENAL THERAPY

(71) Applicant: FRED HUTCHINSON CANCER RESEARCH CENTER, Seattle, WA (US)

(72) Inventors: Richard A. Zager, Mercer Island, WA (US); James M. Olson, Seattle, WA (US); Emily J. Girard, Renton, WA (US); Colin E. Correnti, Seattle, WA (US); Theo L. Sottero, Seattle, WA (US)

(73) Assignee: FRED HUTCHINSON CANCER RESEARCH CENTER, Seattle, WA (US)

(21) Appl. No.: 16/061,179

(22) PCT Filed: Dec. 9, 2016
(86) PCT No.: PCT/US2016/066007

§ 371 Date: Jun. 11, 2018

Related U.S. Application Data
(60) Provisional application No. 62/266,520, filed on Dec. 11, 2015.

(51) Int. Cl. A61K 47/64; A61K 47/54; A61P 13/12

(52) U.S. Cl. CPC A61K 47/6415; A61P 13/12; A61K 47/545; A61K 47/554

(57) ABSTRACT

The present disclosure provides compositions and methods for renal therapy. In various aspects, the present disclosure provides a composition comprising a knotted peptide, wherein upon administration to a subject the knotted peptide homes, targets, is directed to, accumulates in, migrates to, is retained by, and/or binds to renal tissue of the subject. In various aspects, the composition further comprises an active agent coupled to the knotted peptide. The composition, when administered to the subject, may induce protective preconditioning or acquired cytoresistance.

### CROSS REFERENCE

This application claims the benefit of U.S. Provisional Patent Application No. 62/266,520, filed Dec. 11, 2015, the entire content of which is incorporated by reference.

### BACKGROUND

Approximately 9% of the world's population either has, or is expected to develop, chronic renal disease. The leading causes in the United States are diabetic nephropathy and progressive renal dysfunction following a bout of ischemic (e.g., post-cardiac surgery) or toxin-induced (e.g., radiocontrast media, cancer chemotherapy) kidney proximal tubule damage. At present, the US End Stage Renal Disease (ESRD) program consumes ˜7% of the entire Medicare budget. Furthermore, even modest declines in renal function can represent progressive, independent risk factors for rising hospital expenditures, morbidity and mortality. Thus, new ways to protect kidneys, and prophylactically prevent and treat progressive renal diseases are needed.

### SUMMARY

The present disclosure provides compositions and methods for renal therapy.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In various aspects, the present disclosure provides a composition, comprising: a knotted peptide, wherein upon administration to a subject the knotted peptide distributes, homes, targets, migrates to, accumulates in, binds to, is retained by, or is directed to renal tissue of the subject.

In various aspects, the present disclosure provides a composition, comprising: a knotted peptide of any of claims; and a renal therapeutic agent coupled to the knotted peptide.

In some aspects, the knotted peptide comprises a sequence of any one of SEQ ID NO: 1-SEQ ID NO: 59, or a fragment thereof. In other aspects, the knotted peptide comprises a sequence that has at least 80% sequence identity with any one of SEQ ID NO: 1-SEQ ID NO: 59, or a fragment thereof. In still other aspects, the knotted peptide comprises a sequence that has at least 85%, at least 90%, or at least 95% of sequence identity with any one of SEQ ID NO: 1-SEQ ID NO: 59, or a fragment thereof.

In some aspects, the knotted peptide comprises a sequence of any one of SEQ ID NO: 60-SEQ ID NO: 118, or a fragment thereof. In other aspects, the knotted peptide comprises a sequence that has at least 80% sequence identity with any one of SEQ ID NO: 60-SEQ ID NO: 118, or a fragment thereof. In still other aspects, the knotted peptide comprises a sequence that has at least 85%, at least 90%, or at least 95% of sequence identity with any one of SEQ ID NO: 60-SEQ ID NO: 118, or a fragment thereof.

In some aspects, the knotted peptide comprises at least 4, at least 5, at least 6, at least 7, at least 8, at least 9, at least 10, at least 11, at least 12, at least 13, at least 14, at least 15, or at least 16 cysteine residues.

In other aspects, the knotted peptide comprises three or more disulfide bridges formed between cysteine residues, wherein one of the disulfide bridges passes through a loop formed by two other disulfide bridges. In further aspects, the knotted peptide comprises a plurality of disulfide bridges formed between cysteine residues. In still further aspects, the knotted peptide comprises a disulfide through disulfide knot.

In some aspects, at least one amino acid residue of the knotted peptide is in an L configuration or, wherein at least one amino acid residue is in a D configuration.

In some aspects, the sequence comprises at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues.

In some aspects, the knotted peptide comprises or is derived from the group consisting of: chlorotoxins, brazzeins, circulins, stecrisps, hanatoxins, midkines, hefutoxins, potato carboxypeptidase inhibitors, bubble proteins, attractins, α-GI, α-GID, μ-pIIIA, ω-MVIIA, ω-CVID, χ-MrIA, ρ-TIA, conantokin G, contulakin G, GsMTx4, margatoxins, shK, toxin K, chymotrypsin inhibitors (CTI), EGF epiregulin core, hainantoxins, theraphotoxins, hexatoxins, opicalcins, imperatoxins, defensins, and insectotoxins.

In some aspects, the knotted peptide comprises or is derived from a human protein or peptide. In some aspects, the knotted peptide is arranged in a multimeric structure with at least one other knotted peptide.

In further aspects, the multimeric structure comprises a dimer, trimer, tetramer, pentamer, hexamer, or heptamer.

In some aspects, the knotted peptide comprises an isoelectric point less than or equal to about 7.5. In other aspects, the knotted peptide comprises an isoelectric point greater than or equal to about 7.5. In still other aspects, the knotted peptide comprises an isoelectric point within a range from about 3.0 to about 10.0.

In other aspects, the knotted peptide comprises a non-uniform charge distribution. In some aspects, the knotted peptide comprises one or more regions of concentrated positive charge. In other aspects, the knotted peptide comprises one or more regions of concentrated negative charge.

In some aspects, the composition comprises a mass-average molecular weight (Mw) less than or equal to 6 kDa, less than or equal to about 50 kDa, or less than or equal to about 60 kDa. In other aspects, the composition comprises a mass-average molecular weight (Mw) within a range from about 0.5 kDa to about 50 kDa, or within a range from about 0.5 kDa to about 60 kDa.

In some aspects, the knotted peptide is stable at pH values greater than or equal to about 7.0. In other aspects, the knotted peptide is stable at pH values less than or equal to about 5.0, less than or equal to about 3.0, or within a range from about 3.0 to about 5.0. In still other aspects, the knotted peptide is stable at pH values within a range from about 5.0 to about 7.0.

In further aspects, the knotted peptide being stable comprises one or more of: the knotted peptide being capable of performing its therapeutic effect, the knotted peptide being soluble, the knotted peptide being resistant to protease degradation, the knotted peptide being resistant to reduction, the knotted peptide being resistant to pepsin degradation, the knotted peptide being resistant to trypsin degradation, the knotted peptide being reduction resistant, or the knotted peptide being resistant to an elevated temperature.

In some aspects, upon administration to a subject, the knotted peptide homes, targets, is directed to, accumulates in, migrates to, is retained by, or binds to renal tissue of the subject.

In further aspects, the knotted peptide homes, targets, is directed to, accumulates in, migrates to, is retained by, or binds to one or more of: a cortex region, a glomerulus, a proximal tubule, a medulla region, a descending tubule, an ascending tubule, a loop of Henle, or a Bowman's capsule of the subject.

In some aspects, the knotted peptide homes, targets, is directed to, accumulates in, migrates to, is retained by, or binds to a proximal tubule of the subject. In further aspects, the knotted peptide homes, targets, is directed to, accumulates in, migrates to, is retained by, or binds to a cell of the proximal tubule. In still further aspects, the knotted peptide homes, targets, is directed to, accumulates in, migrates to, is retained by, or binds to a cell surface receptor expressed by the cell of the proximal tubule.

In other aspects, the knotted peptide homes, targets, is directed to, accumulates in, migrates to, is retained by, or binds to a glomerulus of the subject. In some aspects, the knotted peptide homes, targets, is directed to, accumulates in, migrates to, is retained by, or binds to a megalin receptor, a cubulin receptor, or a combination thereof. In some aspects, the knotted peptide is internalized by a cell. In some aspects, the knotted peptide is internalized by the cell via a scavenging mechanism.

In some aspects, the knotted peptide exhibits a renal therapeutic effect. In further aspects, the renal therapeutic effect comprises a renal protective effect or renal prophylactic effect.

In some aspects, the knotted peptide interacts with a renal ion channel, inhibits a protease, has antimicrobial activity, has anticancer activity, has anti-inflammatory activity, induces ischemic preconditioning or acquired cytoresistance, or produces a protective or therapeutic effect on a kidney of the subject, or a combination thereof.

In some aspects, at least one residue of the knotted peptide comprises a chemical modification. In further aspects, the chemical modification is blocking the N-terminus of the knotted peptide. In some aspects, the chemical modification is methylation, acetylation, or acylation. In further aspects, the chemical modification is: methylation of one or more lysine residues or analogue thereof; methylation of the N-terminus; or methylation of one or more lysine residue or analogue thereof and methylation of the N-terminus. In other aspects, the knotted peptide is linked to an acyl adduct.

In some aspects, the knotted peptide is linked to an active agent. In further aspects, the active agent is fused with the knotted peptide at an N-terminus or a C-terminus of the knotted peptide. In some aspects, the active agent is an antibody, antibody fragment, or single chain Fv.

In other aspects, the active agent is an Fc domain. In some aspects, the knotted peptide fused with an Fc domain comprises a contiguous sequence.

In some aspects, 1, 2, 3, 4, 5, 6, 7, 8, 9, or 10 active agents are linked to the knotted peptide. In some aspects, the knotted peptide is linked to the active agent via a cleavable linker.

In further aspects, the knotted peptide is linked to the active agent at an N-terminus, at the epsilon amine of an internal lysine residue, at the carboxylic acid of an aspartic acid or glutamic acid residue, or a C-terminus of the knotted peptide by a linker. In still further aspects, the composition comprises a non-natural amino acid, wherein the non-natural amino acid is an insertion, appendage, or substitution for another amino acid.

In some aspects, the knotted peptide is linked to the active agent at the non-natural amino acid by a linker. In some aspects, the linker comprises an amide bond, an ester bond, a carbamate bond, a carbonate bond, a hydrazone bond, an oxime bond, a disulfide bond, a thioester bond, a thioether bond, or a carbon-nitrogen bond. In some aspects, the cleavable linker comprises a cleavage site for matrix metalloproteinases, thrombin, cathepsins, or beta-glucuronidase.

In other aspects, the linker is a hydrolytically labile linker. In still other aspects, the knotted peptide is linked to the active agent via a noncleavable linker.

In some aspects, the active agent is selected from the group consisting of: a peptide, an oligopeptide, a polypeptide, a polynucleotide, a polyribonucleotide, a DNA, a cDNA, a ssDNA, a RNA, a dsRNA, a micro RNA, an oligonucleotide, an antibody fragment, a single chain Fv, an aptamer, a cytokine, an enzyme, a growth factor, a chemokine, a neurotransmitter, a chemical agent, a fluorophore, a metal, a metal chelate, an X-ray contrast agent, a PET agent, a radioisotope, a photosensitizer, a radiosensitizer, a radionuclide chelator, a therapeutic small molecule, a steroid, a corticosteroid, an anti-inflammatory agent, an immune modulator, an immunosuppressant, a protease inhibitor, an amino sugar, a chemotherapeutic, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, a dendrimer, a fatty acid, an Fc region, an iron chelator, a Nrf2 pathway activator, angiotensin-converting-enzyme (ACE) inhibitor, a glycine polymer, a PDGF inhibitor, or an antioxidant.

In some aspects, the iron chelator is deferoxamine. In some aspects, the steroid is dexamethasone or budesonide. In other aspects, the Nrf2 pathway activator is bardoxolone. In some aspects, the ACE inhibitor is enalapril. In still other aspects, the antioxidant is glutathione or N-acetyl cysteine. In some aspects, the NSAID is ketorolac. In other aspects, the NSAID is ibuprofen. In some aspects, the active agent comprises a renal therapeutic agent.

In some aspects, the renal therapeutic agent is selected from the group consisting of: dexamethasone, a steroid, budesonide, triamcinolone acetonide, an anti-inflammatory agent, an antioxidant, deferoxamine, feroxamine, a tin complex, a tin porphyrin complex, a metal chelator, ethylenediaminetetraacetic acid (EDTA), an EDTA-Fe complex, dimercaptosuccinic acid (DMSA), 2,3-dimercapto-1-propanesulfonic acid (DMPS), penicillamine, minocycline, prednisone, azathioprine, mycophenolate mofetil, mycophemolic acid, sirolimius, cyclorsporine, or tacrolimusan antibiotic, an iron chelator, a porphyrin, hemin, vitamin B12, an Nrf2 pathway activator, bardoxolone, ACE inhibitors, enalapril, glycine polymers, antioxidants, glutathione, N acetyl cysteine, a chemotherapeutic, QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an anti-fungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, and retinoic acid.

In some aspects, the iron chelator is deferoxamine. In other aspects, the steroid is dexamethasone or budesonide. In some aspects, the Nrf2 pathway activator is bardoxolone. In other aspects, the ACE inhibitor is enalapril, such as ramipril, captopril, lisinopril, benazepril, quinapril, fosinopril, trandolapril, moexipril, enalaprilat, or perindopril erbumine.

In other aspects, said antibiotic is gentamicin, vancomycin, minocin, or mitomyclin. In some aspects, said immunosuppressant is tacrolimus, mycophenolic acid, cyclosporine A, or azathioprine. In some aspects, the renal therapeutic agent accumulates in the kidney at a higher level when linked to the peptide than when not linked to the peptide.

In other aspects, the renal therapeutic agent comprises a renal protective agent or a renal prophylactic agent. In further aspects, the renal protective agent or renal prophylactic agent is selected from the group consisting of: thiazide, bemetanide, ethacrynic acid, furosemidem torsemide, glucose, mannitol, amiloride, spironolactone, eplerenone, triamterene, potassium canrenoate, bendroflumethiazide, hydrochlorothiazide, vasopressin, amphotericin B, acetazolamide, tovaptan, conivaptan, dopamine, dorzolamide, bendrolumethiazide, hydrochlorothiazide, caffeine, theophylline, theobromine, a statin, a senolytic, navitoclax obatoclax, a corticosteroid, prednisone, betamethasone, fludrocortisone, deoxycorticosterone, aldosterone, cortisone, hydrocortisone, belcometasone, mometasone, fluticasone, prednisolone, methylprednisolone, triamcinolone acetonide, a glucocorticoid, dexamethasone, a steroid, budesonide, triamcinolone acetonide, an anti-inflammatory agent, an antioxidant, a nonsteroidal anti-inflammatory drug (NSAID), deferoxamine, iron, tin, a metal, a metal chelate, ethylenediaminetetraacetic acid (EDTA), dimercaptosuccinic acid (DMSA), 2,3-dimercapto-1-propanesulfonic acid (DMPS), penicillamine, an antibiotic, an aminoglycoside, an iron chelator, a porphyrin, an Nrf2 pathway activator, bardoxolone, ACE inhibitors, enalapril, glycine polymers, antioxidants, glutathione, N acetyl cysteine, a PDGF inhibitor, lithium, ferroptosis inhibitors, vitamin B12QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an anti-fungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, SGLT2 modulator, and retinoic acid.

In some aspects, the iron chelator is deferoxamine. In some aspects, the steroid is dexamethasone or budesonide. In other aspects, the Nrf2 pathway activator is bardoxolone. In some asepects, the ACE inhibitor is enalapril. In some aspects, the NSAID is ketorolac. In other aspects, the NSAID is ibuprofen.

In some aspects, the renal therapeutic agent, renal protective agent, or renal prophylactic agent induces ischemic preconditioning or acquired cytoresistance in a kidney of the subject. In other aspects, the active agent interacts with a renal ion channel, inhibits a protease, has antimicrobial activity, has anticancer activity, has anti-inflammatory activity, induces ischemic preconditioning or acquired cytoresistance, produces a protective or therapeutic effect on a kidney of the subject, reduces a clearance rate of the composition, or a combination thereof.

In some aspects, the composition further comprises a detectable agent coupled to the knotted peptide. In some aspects, the detectable agent is fused with the knotted peptide at an N-terminus or a C-terminus of the knotted peptide. In further aspects, 1, 2, 3, 4, 5, 6, 7, 8, 9, or 10 detectable agents are linked to the knotted peptide.

In further aspects, the knotted peptide is linked to the detectable agent via a cleavable linker. In some aspects, the knotted peptide is linked to the detectable agent at an N-terminus, at the epsilon amine of an internal lysine residue, or a C-terminus of the knotted peptide by a linker.

In other aspects, the composition further comprises a non-natural amino acid, wherein the non-natural amino acid is an insertion, appendage, or substitution for another amino acid. In some aspects, the knotted peptide is linked to the detectable agent at the non-natural amino acid by a linker. In some aspects, the linker comprises an amide bond, an ester bond, a carbamate bond, a hydrazone bond, an oxime bond, or a carbon-nitrogen bond. In further aspects, the cleavable linker comprises a cleavage site for matrix metalloproteinases, thrombin, cathepsins, or beta-glucuronidase.

In other aspects, the knotted peptide is linked to the detectable agent via a noncleavable linker.

In some aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator. In further aspects, the detectable agent is a fluorescent dye.

In some aspects, administration of the composition to a patient mediates inflammation, cell death, fibrosis, or any combination thereof in the kidney.

In various aspects, the present disclosure provides a pharmaceutical composition comprising any one of the above compositions, or a salt thereof, and a pharmaceutically acceptable carrier. In some aspects, the pharmaceutical composition is formulated for administration to a subject. In further aspects, the pharmaceutical composition is formulated for oral administration, intravenous administration, subcutaneous administration, intramuscular administration, or a combination thereof.

In various aspects, the present disclosure provides a method of treating a condition in a subject in need thereof, the method comprising: administering to the subject any one of the compositions or pharmaceutical compositions described above. In some aspects, the composition is administered by inhalation, intranasally, orally, topically, intravenously, subcutaneously, intramuscularly administration, intraperitoneally, or a combination thereof.

In further aspects, the composition or pharmaceutical composition homes, targets, or migrates to renal tissue of the subject following administration.

In some aspects, the condition is associated with a function of the subject's kidneys. In further aspects, the condition is selected from the group consisting of: acute kidney diseases and disorders (AKD), acute kidney injury, acute and rapidly progressive glomerulonephritis, acute presentations of nephrotic syndrome, acute pyelonephritis, acute renal failure, idiopathic chronic glomerulonephritis, secondary chronic glomerulonephritis, chronic heart failure, chronic interstitial nephritis, chronic kidney disease (CKD), chronic liver disease, chronic pyelonephritis, diabetes, diabetic kidney disease, fibrosis, focal segmental glomerulosclerosis, Goodpasture's disease, diabetic nephropathy, hereditary nephropathy, interstitial nephropathy, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, renal fibrosis, graft versus host disease after renal transplant, and vasculitis.

In various aspects, the present disclosure provides a method of protecting a kidney of a subject from injury, the method comprising: administering to the subject the composition or pharmaceutical compositions described above. In some aspects, the composition is administered by inhalation, intranasally, orally, topically, intravenously, subcutaneously, intramuscularly administration, intraperitoneally, or a combination thereof.

In further aspects, the method further comprises inducing ischemic preconditioning or acquired cytoresistance in the kidney of the subject. In some aspects, the injury is associated with one or more of: surgery, radiocontrast imaging, radiocontrast nephropathy, cardiovascular surgery, cardiopulmonary bypass, extracorporeal membrane oxygenation (ECMO), balloon angioplasty, induced cardiac or cerebral ischemic-reperfusion injury, organ transplantation, kidney transplantation, sepsis, shock, low blood pressure, high blood pressure, kidney hypoperfusion, chemotherapy, drug administration, nephrotoxic drug administration, blunt force trauma, puncture, poison, or smoking.

In some aspects, the composition or pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours prior to a predicted occurrence of the injury.

In some aspects, the composition or pharmaceutical composition is administered once per day, week, or month, or once per two weeks, two months, or three months. In other aspects, the composition or pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours after an occurrence of the injury.

In some aspects, the method further comprises performing a medical procedure on the subject. In further aspects, the medical procedure comprises one or more of: surgery, radiocontrast imaging, cardiopulmonary bypass, balloon angioplasty, induced cardiac or cerebral ischemic-reperfusion injury, organ transplantation, chemotherapy, drug administration, or nephrotoxic drug administration.

In some aspects, the composition or the pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours prior to performing the medical procedure.

In other aspects, the composition or the pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours after performing the medical procedure.

In various aspects, the present disclosure provides a method of imaging an organ or body region of a subject, the method comprising: administering to the subject composition of any one of claims 1-103 or a pharmaceutical composition of any one of claims 104-106; and imaging the subject. In further aspects, the method further comprises detecting a cancer or diseased region, tissue, structure or cell.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a brief schematic of a method of manufacturing of a construct that expresses a peptide of the disclosure.

FIG. 2 illustrates a renal signal pattern for a fluoxetine control.

FIG. 3 shows renal signal patterns for a peptide of SEQ ID NO: 4. FIG. 3A shows accumulation of ¹⁴C signal for radiolabeled SEQ ID NO: 4 three hours after peptide administration. FIG. 3B shows accumulation of ¹⁴C signal for a peptide of SEQ ID NO: 4 twenty-four hours after peptide administration.

FIG. 4 shows whole body fluorescence images of mice after administration of SEQ ID NO: 55 conjugated to Cy5.5 (SEQ ID NO: 55-Cy5.5) (left) versus after administration of free Cy5.5-COOH alone (right). FIG. 4A shows a whole body fluorescence image of a mouse 3 hours after administration of 10 nmol SEQ ID NO: 55-Cy5.5. The arrow indicates the position and fluorescence signal in the kidney. FIG. 4B shows a whole body fluorescence image of a mouse 3 hours after administration of 10 nmol Cy5.5-COOH. The arrow indicates the position and fluorescence signal in the kidney. FIG. 4C shows a whole body fluorescence image of a mouse after 24 hours after administration of 10 nmol SEQ ID NO: 55-Cy5.5. The arrow indicates the position and fluorescence signal in the kidney. FIG. 4D shows a whole body fluorescence image of a mouse 24 hours after administration of 10 nmol Cy5.5-COOH. The arrow indicates the position and fluorescence signal in the kidney. FIG. 4E shows a whole body fluorescence image of a mouse 48 hours after administration of 10 nmol SEQ ID NO: 55-Cy5.5. The arrow indicates the position and fluorescence signal in the kidney. FIG. 4F shows a whole body fluorescence image of a mouse 48 hours after administration of 10 nmol Cy5.5-COOH. The arrow indicates the position and fluorescence signal in the kidney. FIG. 4G shows a whole body fluorescence image of a mouse 72 hours after administration of 10 nmol SEQ ID NO: 55-Cy5.5. The arrow indicates the position and fluorescence signal in the kidney. FIG. 4H shows a whole body fluorescence image of a mouse 72 hours after administration of 10 nmol Cy5.5-COOH. The arrow indicates the position and fluorescence signal in the kidney.

FIG. 5 shows fluorescence of kidney sections from mice, in which each mouse received 10 nmol free AlexFluor 647 fluorophore (AF647), 10 nmol SEQ ID NO: 54 conjugated to AF647, 10 nmol SEQ ID NO: 5 conjugated to AF647, or 10 nmol SEQ ID NO: 46 conjugated to AF647. Each kidney was from an independent mouse.

FIG. 6 shows SEQ ID NO: 5 conjugated to AF647 and SEQ ID NO: 54 conjugated to AF647 fluorescence signal in confocal images of the kidney cortex. FIG. 6A shows fluorescence signal of SEQ ID NO: 5 conjugated to AF647 in the kidney cortex 20 hours after of administration of 10 nmol of the peptide-dye conjugate at 6× magnification. FIG. 6B shows fluorescence signal of SEQ ID NO: 5 conjugated to AF647 in the kidney cortex 20 hours after administration of 10 nmol of the peptide-dye conjugate at 20× magnification. FIG. 6C shows fluorescence signal of SEQ ID NO: 5 conjugated to AF647 in the kidney cortex 20 hours after administration of 10 nmol of the peptide-dye conjugate at 6× magnification. FIG. 6D shows fluorescence signal of SEQ ID NO: 5 conjugated to AF647 in the kidney cortex 20 hours after of administration of 10 nmol of the peptide-dye conjugate at 20× magnification.

FIG. 7 shows SEQ ID NO: 46 conjugated to AF647 fluorescence signal in confocal images of the kidney cortex. FIG. 7A shows fluorescence signal of SEQ ID NO: 46 conjugated to AF647 in the kidney cortex 20 hours after administration of 10 nmol of the peptide-dye conjugate at 6× magnification. FIG. 7B shows fluorescence signal of SEQ ID NO: 46 conjugated to AF647 in the kidney cortex 20 hours after administration of 10 nmol of the peptide-dye conjugate at 20× magnification. FIG. 7C shows fluorescence signal in the kidney cortex 20 hours after administration of 10 nmol of a lysozyme-dye conjugate at 6× magnification. FIG. 7D shows fluorescence signal in the kidney cortex 20 hours after of administration of 10 nmol of a lysozyme-dye conjugate at 20× magnification.

FIG. 8 shows the peptide concentration in plasma, urine, and kidney over time. FIG. 8A shows peptide concentration in plasma, urine, and kidney after intravenous administration of 50 nmol of radiolabeled SEQ ID NO: 54 peptide. FIG. 8B shows the peptide concentration in plasma, urine, and kidney after intravenous administration of 50 nmol of radiolabeled peptide of SEQ ID NO: 5. FIG. 8C shows the peptide concentration in plasma, urine, and kidney after intravenous administration of 50 nmol of a radiolabeled peptide of SEQ ID NO: 46.

FIG. 9 shows the peptide concentration in plasma, urine, or kidney over time. FIG. 9A shows the peptide concentration in plasma after intravenous administration of 50 nmol radiolabeled SEQ ID NO: 54, 50 nmol radiolabeled SEQ ID NO: 5, or 50 nmol radiolabeled SEQ ID NO: 46. FIG. 9B shows the peptide concentration in urine after intravenous administration of 50 nmol radiolabeled SEQ ID NO: 54, 50 nmol radiolabeled SEQ ID NO: 5, or 50 nmol radiolabeled SEQ ID NO: 46 in urine. FIG. 9C shows the peptide concentration in kidney after intravenous administration of 50 nmol radiolabeled SEQ ID NO: 54, radiolabeled SEQ ID NO: 5, or radiolabeled SEQ ID NO: 46.

FIG. 10 shows quantified fluorescence signal, indicating renal uptake, of a peptide of SEQ ID NO: 4 conjugated to AlexaFluor647 (AF647) and an unlabeled SEQ ID NO: 4 peptide 4 hours after intravenous administration of 2 nmol of SEQ ID NO: 4-AF647, 10 nmol of SEQ ID NO: 4 (1:5) co-injected with 2 nmol of SEQ ID NO: 4-AF647 (5:1), or 50 nmol of SEQ ID NO: 4 co-injected with 2 nmol of SEQ ID NO: 4-AF647 (25:1). Kidneys from uninjected mice were used as a negative control.

FIG. 11 shows quantified fluorescence signal, indicating renal uptake, between a peptide of SEQ ID NO: 4 conjugated to AlexaFluor647 (AF647) and unlabeled KKEEEKKEEEKKEEEKK competitor peptide (SEQ ID NO: 121, a known renal targeting peptide) 1 hour after intravenous administration of 2 nmol of a peptide of SEQ ID NO: 4-AF647, 2 nmol of a peptide of SEQ ID NO: 4-AF647 co-injected with 100 nmol of an unlabeled peptide of SEQ ID NO: 121 (1:50), or 2 nmol of peptide of SEQ ID NO: 4-AF647 co-injected with 2000 nmol of an unlabeled peptide of SEQ ID NO: 121 (1:1000).

FIG. 12 shows quantified fluorescence signal, indicating renal uptake, between a peptide of SEQ ID NO: 4 conjugated to AlexaFluor647 (AF647) and a control peptide conjugated to AF647 (control peptide-AF647), 4 hours after intravenous administration of 10 nmol of a peptide of SEQ ID NO: 4-AF647 or 10 nmol of a peptide of control peptide-AF647.

FIG. 13 shows fluorescence signal in the kidneys 30 minutes after administration of either 10 nmol free AF647 fluorophore or 10 nmol SEQ ID NO: 4 conjugated to AF647 (SEQ ID NO: 4-AF647). Kidneys were isolated, sectioned, and imaged using a Zeiss confocal microscopy.

FIG. 13A shows fluorescence signal from free AF647 fluorophore at 10× magnification. FIG. 13B shows fluorescence signal of SEQ ID NO: 4-AF647 at 40× magnification.

FIG. 14 shows fluorescence signal in the kidney 30 minutes after administration of 10 nmol SEQ ID NO: 46 conjugated to AF647 (SEQ ID NO: 46-AF647). Kidneys were isolated, sectioned, and imaged using a Zeiss confocal microscope. FIG. 14A shows fluorescence signal at 10× magnification. FIG. 14B shows fluorescence signal at 40× magnification.

FIG. 15 shows stability of SEQ ID NO: 5 peptide. FIG. 15A shows the HPLC chromatogram of a non-reduced peptide of SEQ ID NO: 5. FIG. 15B shows an SDS-PAGE of a non-reduced (Lane 2) and reduced (Lane 3) of a peptide of SEQ ID NO: 5.

FIG. 16 shows stability of SEQ ID NO: 46 peptide. FIG. 16A shows the HPLC chromatogram of a non-reduced peptide of SEQ ID NO: 46. FIG. 16B shows an SDS-PAGE of a non-reduced (Lane 2) and reduced (Lane 3) of a peptide of SEQ ID NO: 46.

FIG. 17 shows stability of SEQ ID NO: 54 peptide. FIG. 17A shows the HPLC chromatogram of a non-reduced peptide of SEQ ID NO: 54. FIG. 17B shows an SDS-PAGE of a non-reduced (Lane 2) and reduced (Lane 3) of a peptide of SEQ ID NO: 54.

FIG. 18 shows stability of SEQ ID NO: 55 peptide. FIG. 18A shows the HPLC chromatogram of a non-reduced peptide of SEQ ID NO: 55. FIG. 18B shows an SDS-PAGE of a non-reduced (Lane 2) and reduced (Lane 3) of a peptide of SEQ ID NO: 55.

FIG. 19 shows stability of SEQ ID NO: 4 peptide. FIG. 19A shows the HPLC chromatogram of a non-reduced peptide of SEQ ID NO: 4. FIG. 19B shows an SDS-PAGE of a non-reduced (Lane 2) and reduced (Lane 3) of a peptide of SEQ ID NO: 4.

FIG. 20 shows stability of SEQ ID NO: 56 peptide. FIG. 20A shows the HPLC chromatogram of a non-reduced peptide of SEQ ID NO: 56. FIG. 20B shows an SDS-PAGE of a non-reduced (Lane 2) and reduced (Lane 3) of a peptide of SEQ ID NO: 56.

FIG. 21 shows stability of SEQ ID NO: 57 peptide. FIG. 21A shows the HPLC chromatogram of a non-reduced peptide of SEQ ID NO: 57. FIG. 21B shows an SDS-PAGE of a non-reduced (Lane 2) and reduced (Lane 3) of a peptide of SEQ ID NO: 57.

FIG. 22 shows stability of SEQ ID NO: 58 peptide. FIG. 22A shows the HPLC chromatogram of a non-reduced peptide of SEQ ID NO: 58. FIG. 22B shows an SDS-PAGE of a non-reduced (Lane 2) and reduced (Lane 3) of a peptide of SEQ ID NO: 58.

FIG. 23 shows stability of SEQ ID NO: 59 peptide. FIG. 23A shows the HPLC chromatogram of a non-reduced peptide of SEQ ID NO: 59. FIG. 23B shows an SDS-PAGE of a non-reduced (Lane 2) and reduced (Lane 3) of a peptide of SEQ ID NO: 59.

FIG. 24 shows mice had normal renal physiology 24 hours after intravenous administration of 100 nmol of a peptide of SEQ ID NO: 18, SEQ ID NO: 20, SEQ ID NO: 21, SEQ ID NO: 26, SEQ ID NO: 36, SEQ ID NO: 39, SEQ ID NO: 45, or SEQ ID NO: 53, or a PBS injected negative control. The kidneys were stained using periodic acid Schiff (PAS).

### DETAILED DESCRIPTION

The present disclosure relates generally to compositions and methods for renal therapy. In some embodiments, the compositions and methods herein utilize peptides that can home, target, are directed to, accumulate in, migrate to, are retained by and/or bind to the kidneys following administration to a subject. In certain embodiments, the peptides described herein can bind to or accumulate in a specific region, tissue, structure, or cell of a kidney, e.g., the proximal tubule, the glomerulus, or the glomerular filtrate (Bowman's space) tubular lumina. The properties of the peptide (e.g., isoelectric point (pI), molecular weight, pH stability, reduction resistance, protease resistance, hydrophobicity/hydrophilicity, charge, etc.) can be selected to provide improved renal localization and binding. In some embodiments, the renal homing peptides of the present disclosure are used to deliver an active agent to the kidney or a tissue, region, compartment or cell thereof. The active agent can exert a therapeutic effect on the kidney or a tissue or cell thereof. For example, in certain embodiments, the active agent induces a protective response such as ischemic preconditioning or acquired cytoresistance in the kidney or tissue or cell thereof. As another example, in certain embodiments, the active agent induces a therapeutic response in a diseased kidney or tissue, region, compartment or cell thereof. In certain embodiments, the peptide itself induces such protective and therapeutic responses, such as by binding to ion channels, exerting an antimicrobial effect, or inhibiting protease(s).

Iron (Fe) mediated oxidative stress and renal interstitial inflammation can lead to progressive nephron loss and renal interstitial fibrosis. The severity of the latter, as assessed on kidney biopsy, can be a predictor of subsequent loss of renal function. Despite recognition of their pathogenic roles, therapies targeted at Fe-mediated oxidative stress and renal inflammation have been hampered by two dominant factors: 1) an inability to achieve sufficient intrarenal concentrations of potent antioxidant/Fe binding agents (e.g., deferoxamine); and 2) associated systemic toxicities (e.g., with glucocorticoids, cyclophosphamide therapies). A molecule that can distribute sufficient levels of a therapeutic agent to the kidney while reducing the levels of the agent delivered to other areas of the body such as to reduce off-target toxicities may be able to achieve a therapeutic window that allows treatment of the kidney with the agent with a sufficient safety profile. Likewise, an active molecule that can accumulate in the kidney with reduced distribution to other tissues may be able to achieve a therapeutic effect in the kidney while sufficiently sparing other tissues from side effects. For example, steroid treatment of the kidney can be limited by toxicity side effects in other parts of the body and in particular, can be contraindicated in diabetic patients due to off-target toxicities.

In some embodiments, the present disclosure sets forth pro-drugs that specifically target the kidney. In some cases, low molecular weight proteins in plasma (LMWPs; <35 kDa) can be freely filtered by the glomerulus, and can be almost fully reabsorbed by proximal tubules (which represent ˜70% of total renal cortical mass). The reabsorbed protein can be degraded within the proximal tubular lysosomal system. Thus, by binding small therapeutic molecules to a specific LMWP, the bound agent(s) can be tunably released from its carrier protein within tubular cells, gaining access to the tubular cytosol, and subsequently, the renal interstitial compartment (the dominant site of the renal inflammatory response).

The present disclosure provides a number of peptides that can be rapidly, highly, and persistently taken up by or can accumulate in proximal tubule cells or in the glomerular filtrate (Bowman's space) tubular lumina. These peptides can prevent and treat a host of acute and progressive renal diseases or can be linked to a small therapeutic molecule that can prevent and treat a host of acute and progressive renal diseases. Given that many renal diseases, both acute and chronic, can be mediated in large part by both inflammation and iron mediated oxidative stress, the peptide-drug conjugates of the present disclosure can be applicable in a wide range of clinical settings.

The peptides disclosed herein also can provide several advantages over other known approaches for treatment of acute or progressive renal disease. For example, a peptide of this disclosure can deliver molecules intracellularly, and thus act on intracellular targets as compared to other approaches. Additionally, as compared to treatment using lysozyme or myoglobin, a peptide of the disclosure can have reduced immunogenicity, be soluble in kidney compartments, have a lack of toxicity or reduced toxicity to kidney, and can be resistant to reduction and/or to proteases. A peptide as disclosed herein can also have a controlled and/or single site for drug conjugation as compared to other known treatments. For example, both a lysozyme and another previously known kidney targeting peptide, KKEEEKKEEEKKEEEKK (SEQ ID NO: 121), can comprise multiple lysine residues as compared with a peptide of the disclosure, such as SEQ ID NO: 54-SEQ ID NO: 59, which have been or can be engineered to have no lysine residue. The absence of a lysine residue on a peptide of the disclosure can allow for site specific amine conjugation at the N-terminus of the peptide or can allow for a single lysine residue to be a site specific conjugation. Furthermore, lysozyme can have cardiovascular side effects in comparison with a peptide of this disclosure.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R). D amino acids are denoted with lower case letters.

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

### Peptides

In some embodiments, the present disclosure provides peptides that comprise or are derived from knotted peptides. As used herein, the term “knotted peptide” is considered to be interchangeable with the terms “knottin” and “peptide.” Knotted peptides are a class of peptides, usually ranging from about 11 to about 81 amino acids in length, that are often folded into a compact structure. In certain embodiments, knotted peptides are assembled into a complex tertiary structure that is characterized by a number of intramolecular disulfide crosslinks, and optionally contain beta strands and other secondary structures such as an alpha helix.

The peptides of the present disclosure can comprise cysteine amino acid residues. In some cases, the peptide has at least 4 cysteine amino acid residues. In some cases, the peptide has at least 6 cysteine amino acid residues. In other cases, the peptide has at least 8 cysteine amino acid residues, at least 10 cysteine amino acid residues, at least 12 cysteine amino acid residues, at least 14 cysteine amino acid residues or at least 16 cysteine amino acid residues.

For example, knotted peptides include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include, e.g., growth factor cysteine knots or inhibitor cysteine knots. Other possible peptide structures include peptide having two parallel helices linked by two disulfide bridges without β-sheets (e.g., hefutoxin). The presence of the disulfide bonds can give knottins remarkable environmental stability, allowing them to withstand extremes of temperature and pH and to resist the proteolytic enzymes of the blood stream and of the digestive tract.

A wider examination of the sequence structure and homology of knottins reveals that they have arisen by convergent evolution in all kinds of animals and plants. In animals, they are can be found in venoms, for example, the venoms of spiders and scorpions and have been implicated in the modulation of ion channels. Many of this class of peptide can be protease inhibitors, and as such can both home to kidneys. The knottin proteins of plants can inhibit the proteolytic enzymes of animals or have antimicrobial activity, suggesting that knottins can function in the native defense of plants.

The knotted peptides of the present disclosure can provide certain advantages. For instance, the presence of the disulfide bonds in a knotted structure can give a peptide remarkable environmental stability, allowing it to withstand extremes of temperature and pH and to resist the proteolytic enzymes of the blood stream, the gastrointestinal tract, and elsewhere in the body, and to resist reduction such as by glutathione inside a cell. The resistance of knotted peptides to degradation can be beneficial in terms of reducing immunogenicity. The rigidity of knotted peptides also can allow them to bind to targets without paying the “entropic penalty” that a floppy peptide can accrue upon binding a target (e.g., in renal tissue) compared to other types of molecules.

A knotted peptide can comprise at least one amino acid residue in an L configuration. A knotted peptide can comprise at least one amino acid residue in a D configuration. In some embodiments, a knotted peptide is 11-81 amino acid residues long. In some embodiments, a knotted peptide is 22-63 amino acid residues long. In some embodiments, a knotted peptide is 15-40 amino acid residues long. In other embodiments, a knotted peptide is 11-57 amino acid residues long. In further embodiments, a knotted peptide is at least 20 amino acid residues long.

In some embodiments, the peptides of the present disclosure (e.g., knotted peptides) are derived from a class of proteins known to be present or associated with toxins or venoms. In certain embodiments, the peptide is derived from toxins or venoms associated with scorpions or spiders. A peptide can be derived from venoms and toxins of spiders and scorpions of various genus and species. For example, a peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Grammostola rosea, Haplopelma hainanum, or another suitable genus or species of scorpion or spider. In certain embodiments, a peptide is derived from a Buthus martensii Karsh (scorpion) toxin.

In some embodiments, the peptides of the present disclosure comprise or are derived from one or more of the following: chlorotoxins, brazzeins, circulins, stecrisps, hanatoxins, midkines, hefutoxins, potato carboxypeptidase inhibitors, bubble proteins, attractins, α-GI, α-GID, μ-pIIIA, ω-MVIIA, ω-CVID, χ-MrIA, ρ-TIA, conantokin G, contulakin G, GsMTx4, margatoxins, shK, toxin K, chymotrypsin inhibitors (CTI), EGF epiregulin core, hainantoxins, theraphotoxins, hexatoxins, opicalcins, imperatoxins, defensins, or insectotoxins.

In certain embodiments, the peptides of the present disclosure comprise or are derived from a human protein or peptide that comprises a knotted peptide. Examples of such human proteins or peptides include but are not limited to: bone morphogenic protein 7, gremlin, Cerberus, human chorionic gonadotrophin (hCG), AgRP, siderocalin, receptor-associated protein (RAP), ANKRA2, LRP2BP, DAB2, lactoferrin, and other known megalin/cubulin interactors. Optionally, the human proteins or peptides provided herein are used for motif grafting onto knotted peptide scaffolds.

In alternative embodiments, the peptides of the present disclosure comprise or are derived from a non-human protein or peptide that comprises a knotted peptide, but are modified to include amino acid sequences found in human proteins or peptides. Such modifications can be performed in order to enable binding to human targets (e.g., grafting a known epitope from a human protein that binds to the megalin/cubulin receptor in order to promote proximal tubule binding).

The present disclosure further includes peptide scaffolds that can be used as a starting point for generating additional peptides. In some embodiments, these scaffolds are derived from a variety of knotted peptides or knottins. Some suitable peptides for scaffolds can include, but are not limited to, chlorotoxin, brazzein, circulin, stecrisp, hainantoxin, midkine, hefutoxin, potato carboxypeptidase inhibitor, bubble protein, attractin, α-GI, α-GID, μ-PIIIA, ω-MVIIA, ω-CVID, χ-MrIA, ρ-TIA, conantokin G, contulakin G, GsMTx4, margatoxin, shK, toxin K, chymotrypsin inhibitor (CTI), and EGF epiregulin core. In certain embodiments, the present disclosure relates to knotted peptides that can include 15 to 40, or in some embodiments 22 to 63, amino acid disulfide-linked peptides as potential drug scaffolds.

In some embodiments, the peptides of the present disclosure comprise one or more cysteine amino acid residues. In certain embodiments, the peptide comprises at least 4, at least 5, at least 6, at least 7, at least 8, at least 9, at least 10, at least 11, at least 12, at least 13, at least 14, at least 15, or at least 16 cysteine residues.

A knotted peptide can comprise disulfide bridges. A knotted peptide can be a peptide wherein 5% or more of the residues are cysteines forming intramolecular disulfide bonds. A disulfide-linked peptide can be a drug scaffold. In some embodiments, the peptides of the present disclosure comprise a plurality of disulfide bridges forming an inhibitor knot. In certain embodiments, the disulfide bridges are formed between cysteine residues of the peptide. For example, in various embodiments, the 1^{st} cysteine residue in the sequence is disulfide bonded with the 4^{th} cysteine residue in the sequence, the 2^{nd} cysteine residue in the sequence is disulfide bonded with the 5^{th} cysteine residue in the sequence, and/or the 3^{rd} cysteine residue in the sequence is disulfide bonded with the 6^{th} cysteine residue in the sequence. In alternative embodiments, the disulfide bridges can be formed between any two cysteine residues. In some cases, one disulfide bridge passes through a loop or ring formed by two other disulfide bridges, for example, to form a disulfide through disulfide knot (e.g., an inhibitor knot), also known as a “two-and-through” system.

In some embodiments, the peptide contains one or more disulfide bonds and has a positive net charge at neutral pH, where the net charge of the peptide is greater than or equal to 0 and less than or equal to +30 or where the net charge of the peptide is greater than or equal to −30 and less than or equal to 0. For example, in some embodiments, the peptide has a positive net charge at neutral pH, where the net charge is +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10, +11 or less than +11, +12 or less than +12, +13 or less than +13, +14 or less than +14, +15 or less than +15, +16 or less than +16, +17 or less than +17, +18 or less than +18, +19 or less than +19, +20 or less than +20, +21 or less than +21, +22 or less than +22, +23 or less than +23, +24 or less than +24, +25 or less than +25, +26 or less than +26, +27 or less than +27, +28 or less than +28, +29 or less than +29, or +30 or less than +30. In some embodiments, the peptide has a negative net charge at neutral pH, where the net charge is −0.5 or more than −0.5, −1 or more than −1, −1.5 or more than −1.5, −2 or more than −2, −2.5 or more than −2.5, −3 or more than −3, −3.5 or more than −3.5, −4 or more than −4, −4.5 or more than −4.5, −5 or more than −5, −5.5 or more than −5.5, −6 or more than −6, −6.5 or more than −6.5, −7 or more than −7, −7.5 or more than −7.5, −8 or more than −8, −8.5 or more than −8.5, −9 or more than −9.5, −10 or more than −10, −11 or more than −11, −12 or more than −12, −13 or more than −13, −14 or more than −14, −15 or more than −15, −16 or more than −16, −17 or more than −17, −18 or more than −18, −19 or more than −19, −20 or more than −20, −21 or more than −21, −22 or more than −22, −23 or more than −23, −24 or more than −24, −25 or more than −25, −26 or more than −26, −27 or more than −27, −28 or more than −28, −29 or more than −29, or −30 or more than −30.

In various embodiments, the peptides of the present disclosure comprise positively charged amino acid residues. In some embodiments, the peptide has at least 1 positively charged residue, at least 2 positively charged residues, at least 3 positively charged residues, at least 4 positively charged residues, at least 5 positively charged residues, at least 6 positively charged residues, at least 7 positively charged residues, at least 8 positively charged residues, at least 9 positively charged residues, at least 10 positively charged residues, at least 11 positively charged residues, at least 12 positively charged residues, at least 13 positively charged residues, at least 14 positively charged residues, at least 15 positively charged residues, at least 16 positively charged residues, or at least 17 positively charged residues. While the positively charged residues can be selected from any positively charged amino acid residues, in certain embodiments, the positively charged residues are either K, or R or a combination of K and R.

In various embodiments, the peptides of the present disclosure comprise negative amino acid residues. In some embodiments, the peptide has 1 or fewer negative amino acid residues, 2 or fewer negative amino acid residues, 3 or fewer negative amino acid residues, or 4 or fewer negative amino acid residues, 5 or fewer negative amino acid residues, 6 or fewer negative amino acid residues, 7 or fewer negative amino acid residues, 8 or fewer negative amino acid residues, 9 or fewer negative amino acid residues, or 10 or fewer negative amino acid residues. While negative amino acid residues can be selected from any negative charged amino acid residues, in certain embodiments, the negative amino acid residues are either E, or D or a combination of both E and D.

In various embodiments, the peptides of the present disclosure comprise neutral amino acid residues. In some embodiments, the peptide has 1 or fewer neutral amino acid residues, 2 or fewer neutral amino acid residues, 3 or fewer neutral amino acid residues, 4 or fewer neutral amino acid residues, 5 or fewer neutral amino acid residues, 6 or fewer neutral amino acid residues, 7 or fewer neutral amino acid residues, 8 or fewer neutral amino acid residues, 9 or fewer neutral amino acid residues, 10 or fewer neutral amino acid residues, 15 or fewer neutral amino acid residues, 20 or fewer neutral amino acid residues, 25 or fewer neutral amino acid residues, 30 or fewer neutral amino acid residues, 35 or fewer neutral amino acid residues, 40 or fewer neutral amino acid residues, or 60 or fewer neutral amino acid residues.

TABLE 1 lists exemplary peptides according to the present disclosure.

TABLE 1
Exemplary Peptides.

| SEQ ID NO | Amino Acid Sequence |
|---|---|
| SEQ ID NO: 1 | GSDCLPHLRRCRADNDCCGRRCRRRGTNAERRCR |
| SEQ ID NO: 2 | GSDCKYKFENWGACDGGTGTKVRQGTLKKARYNAQCQETIRVTKPC |
| SEQ ID NO: 3 | GSDCKYKFENWGACDGGTGTKVRQGTLKKARYNAQCQETIRVTKPCTP |
| | KTKAKAKAKKGKGKD |
| SEQ ID NO: 4 | GSSCEPGRTFRDRCNTCRCGADGRSAACTLRACPNQ |
| SEQ ID NO: 5 | GSQFTNVSCTTSRECWSVCQRLHNTSRGRCMNRRCRCYS |
| SEQ ID NO: 6 | GSMCMPCFTTDHQMARRCDDCCGGRGRGRCYGPQCLCR |
| SEQ ID NO: 7 | GSISIGIKCSPSIDLCEGQCRIRKYFTGYCSGDTCHCSG |
| SEQ ID NO: 8 | GSEVIRCSGSKQCYGPCKQQTGCTNSKCMNKVCKCYGCG |
| SEQ ID NO: 9 | GSSEKDCIKHLQRCRENKDCCSKKCSRRGTNPEKRCR |
| SEQ ID NO: 10 | GSSCAKPRENCNRMNILCCRGECVCPTFGDCFCYGD |
| SEQ ID NO: 11 | GSGVPINVKCRGSRDCLDPCKKAGMRFGKCINSKCHCTP |
| SEQ ID NO: 12 | GSVRIPVSCKHSGQCLKPCKDAGMRFGKCMNGKCDCTPK |
| SEQ ID NO: 13 | GSGIVCKVCKIICGMQGKKVNICKApIKCKCKKG |
| SEQ ID NO: 14 | GSDCVRFWGKCSQTSDCCPHLACKSKWPRNICVWDGSVG |
| SEQ ID NO: 15 | GSAVCVYRTCDKDCKRRGYRSGKCINNACKCYPYG |
| SEQ ID NO: 16 | GSGCFGYKCDYYKGCCSGYVCSPTWKWCVRPGPGR |
| SEQ ID NO: 17 | GSQVQTNVKCQGGSCASVCRREIGVAAGKCINGKCVCYRN |
| SEQ ID NO: 18 | GSGDCLPHLKRCKENNDCCSKKCKRRGANPEKRCR |
| SEQ ID NO: 19 | GSNFKVEGACSKPCRKYCIDKGARNGKCINGRCHCYY |
| SEQ ID NO: 20 | GSQKILSNRCNNSSECIPHCIRIFGTRAAKCINRKCYCYP |
| SEQ ID NO: 21 | GSDRDSCIDKSRCSKYGYYQECQDCCKKAGHNGGTCMFFKCKCA |
| SEQ ID NO: 22 | GSAVCNLKRCQLSCRSLGLLGKCIGDKCECVKHG |
| SEQ ID NO: 23 | GSQFCGTNGKPCVNGQCCGALRCVVTYHYADGVCLKMNP |
| SEQ ID NO: 24 | GSRPTDIKCSASYQCFPVCKSRFGKTNGRCVNGLCDCF |
| SEQ ID NO: 25 | GSNCAGYMRECKEKLCCSGYVCSSRWKWCVLPAPWRR |
| SEQ ID NO: 26 | GSQFTDVKCTGSKQCWPVCKQMFGKPNGKCMNGKCRCYS |
| SEQ ID NO: 27 | GSQIDTNVKCSGSSKCVKICIDRYNTRGAKCINGRCTCYP |
| SEQ ID NO: 28 | GSAEIIRCSGTRECYAPCQKLTGCLNAKCMNKACKCYGCV |
| SEQ ID NO: 29 | GSSDYCSNDFCFFSCRRDRCARGDCENGKCVCKNCHLN |
| SEQ ID NO: 30 | GSCIGEGVPCDENDPRCCFGLVCLKPTLHGIWYKSYYCYKK |
| SEQ ID NO: 31 | GSSCAKPGEMCMRIKCCDGQCGCNRGTGRCFCK |
| SEQ ID NO: 32 | GSACLAEYQKCEGSTVPCCPGLSCSAGRFRKTKLCTK |
| SEQ ID NO: 33 | GSVVIGQRCYRSPDCYSACKKLVGKATGKCTNGRCDC |
| SEQ ID NO: 34 | GSACQFWSCNSSCISRGYRQGYCWGIQYKYCQCQ |
| SEQ ID NO: 35 | GSRCPPCFTTNPNMEADCRKCCGGRGYCASYQCICPGG |
| SEQ ID NO: 36 | GSVFINVKCRGSPECLPKCKEAIGKSAGKCMNGKCKCYP |
| SEQ ID NO: 37 | GSQVSTNKKCSNTSQCYKTCEKVVGVAAGKCMNGKCICYP |
| SEQ ID NO: 38 | GSECLEIFKACNPSNDQCCKSSKLVCSRKTRWCKYQIG |
| SEQ ID NO: 39 | GSQDKCKKVYENYPVSKCQLANQCNYDCKLDKHARSGECFYDEKRNL |
| | QCICDYCEY |
| SEQ ID NO: 40 | GSGHACYRNCWREGNDEETCKERC |
| SEQ ID NO: 41 | GSMCMPCFTTDTQMQERCDRCCGGGGRGRCWGPQCLCI |
| SEQ ID NO: 42 | GSMCMPCFTTEQRMAIICDDCCGGFGRGRCYGPQCLCR |
| SEQ ID NO: 43 | GSICIPCFTTDHQIARRCDDCCGGRGRGRCYGPQCICR |
| SEQ ID NO: 44 | GSRCQLQGFNCVVRSYGLPTIPCCRGLTCRSYFPGSTYGRCQRY |
| SEQ ID NO: 45 | GSSFGLCRLRRGFCARGRCRFPSIPIGRCSRFVQCCRRVW |
| SEQ ID NO: 46 | GSSCEPGTTFRDRCNTCRCGSDGRSAACTLRACPQ |
| SEQ ID NO: 47 | GSSCTPGTTFRDRCNTCRCSSNGRSAACTLRACPPGSY |
| SEQ ID NO: 48 | GSSCTPGTTFRNRCNTCRCGSNGRSASCTLMACPPGSY |
| SEQ ID NO: 49 | GSSCTPGATFRNRCNTCRCGSNGRSASCTLMACPPGSY |
| SEQ ID NO: 50 | GSSCQPGTTYQRGCNTCRCLEDGQTEACTLRLC |
| SEQ ID NO: 51 | GSSCTPGATYREGCNICRCRSDGRSGACTRRICPVDSN |
| SEQ ID NO: 52 | GSSCQPGTTFRRDCNTCVCNRDGTNAACTLRACL |
| SEQ ID NO: 53 | GGYSRCQLQGFNCVVRSYGLPTIPCCRGLTCRSYFPGSTYGRCQRY |
| SEQ ID NO: 54 | GSSCARPRENCNRMNILCCRGECVCPTFGDCFCYGD |
| SEQ ID NO: 55 | GSGVPINVRCRGSRDCLDPCRRAGMRFGRCINSRCHCTP |
| SEQ ID NO: 56 | GSSERDCIRHLQRCRENRDCCSRRCSRRGTNPERRCR |
| SEQ ID NO: 57 | GSVRIPVSCRHSGQCLRPCRDAGMRFGRCMNGRCDCTPR |
| SEQ ID NO: 58 | GSQVQTNVRCQGGSCASVCRREIGVAAGRCINGRCVCYRN |
| SEQ ID NO: 59 | GSGDCLPHLRRCRENNDCCSRRCRRRGANPERRCR |
| SEQ ID NO: 60 | DCLPHLRRCRADNDCCGRRCRRRGTNAERRCR |
| SEQ ID NO: 61 | DCKYKFENWGACDGGTGTKVRQGTLKKARYNAQCQETIRVTKPC |
| SEQ ID NO: 62 | DCKYKFENWGACDGGTGTKVRQGTLKKARYNAQCQETIRVTKPCTPK |
| | TKAKAKAKKGKGKD |
| SEQ ID NO: 63 | SCEPGRTFRDRCNTCRCGADGRSAACTLRACPNQ |
| SEQ ID NO: 64 | QFTNVSCTTSRECWSVCQRLHNTSRGRCMNRRCRCYS |
| SEQ ID NO: 65 | MCMPCFTTDHQMARRCDDCCGGRGRGRCYGPQCLCR |
| SEQ ID NO: 66 | ISIGIKCSPSIDLCEGQCRIRKYFTGYCSGDTCHCSG |
| SEQ ID NO: 67 | EVIRCSGSKQCYGPCKQQTGCTNSKCMNKVCKCYGCG |
| SEQ ID NO: 68 | SEKDCIKHLQRCRENKDCCSKKCSRRGTNPEKRCR |
| SEQ ID NO: 69 | SCAKPRENCNRMNILCCRGECVCPTFGDCFCYGD |
| SEQ ID NO: 70 | GVPINVKCRGSRDCLDPCKKAGMRFGKCINSKCHCTP |
| SEQ ID NO: 71 | VRIPVSCKHSGQCLKPCKDAGMRFGKCMNGKCDCTPK |
| SEQ ID NO: 72 | GIVCKVCKIICGMQGKKVNICKApIKCKCKKG |
| SEQ ID NO: 73 | DCVRFWGKCSQTSDCCPHLACKSKWPRNICVWDGSVG |
| SEQ ID NO: 74 | AVCVYRTCDKDCKRRGYRSGKCINNACKCYPYG |
| SEQ ID NO: 75 | GCFGYKCDYYKGCCSGYVCSPTWKWCVRPGPGR |
| SEQ ID NO: 76 | QVQTNVKCQGGSCASVCRREIGVAAGKCINGKCVCYRN |
| SEQ ID NO: 77 | GDCLPHLKRCKENNDCCSKKCKRRGANPEKRCR |
| SEQ ID NO: 78 | NFKVEGACSKPCRKYCIDKGARNGKCINGRCHCYY |
| SEQ ID NO: 79 | QKILSNRCNNSSECIPHCIRIFGTRAAKCINRKCYCYP |
| SEQ ID NO: 80 | DRDSCIDKSRCSKYGYYQECQDCCKKAGHNGGTCMFFKCKCA |
| SEQ ID NO: 81 | AVCNLKRCQLSCRSLGLLGKCIGDKCECVKHG |
| SEQ ID NO: 82 | QFCGTNGKPCVNGQCCGALRCVVTYHYADGVCLKMNP |
| SEQ ID NO: 83 | RPTDIKCSASYQCFPVCKSRFGKTNGRCVNGLCDCF |
| SEQ ID NO: 84 | NCAGYMRECKEKLCCSGYVCSSRWKWCVLPAPWRR |
| SEQ ID NO: 85 | QFTDVKCTGSKQCWPVCKQMFGKPNGKCMNGKCRCYS |
| SEQ ID NO: 86 | QIDTNVKCSGSSKCVKICIDRYNTRGAKCINGRCTCYP |
| SEQ ID NO: 87 | AEIIRCSGTRECYAPCQKLTGCLNAKCMNKACKCYGCV |
| SEQ ID NO: 88 | SDYCSNDFCFFSCRRDRCARGDCENGKCVCKNCHLN |
| SEQ ID NO: 89 | CIGEGVPCDENDPRCCFGLVCLKPTLHGIWYKSYYCYKK |
| SEQ ID NO: 90 | SCAKPGEMCMRIKCCDGQCGCNRGTGRCFCK |
| SEQ ID NO: 91 | ACLAEYQKCEGSTVPCCPGLSCSAGRFRKTKLCTK |
| SEQ ID NO: 92 | VVIGQRCYRSPDCYSACKKLVGKATGKCTNGRCDC |
| SEQ ID NO: 93 | ACQFWSCNSSCESRGYRQGYCWGIQYKYCQCQ |
| SEQ ID NO: 94 | RCPPCFTTNPNMEADCRKCCGGRGYCASYQCICPGG |
| SEQ ID NO: 95 | VFINVKCRGSPECLPKCKEAIGKSAGKCMNGKCKCYP |
| SEQ ID NO: 96 | QVSTNKKCSNTSQCYKTCEKVVGVAAGKCMNGKCICYP |
| SEQ ID NO: 97 | ECLEIFKACNPSNDQCCKSSKLVCSRKTRWCKYQIG |
| SEQ ID NO: 98 | QDKCKKVYENYPVSKCQLANQCNYDCKLDKHARSGECFYDEKRNLQC |
| | ICDYCEY |
| SEQ ID NO: 99 | GHACYRNCWREGNDEETCKERC |
| SEQ ID NO: 100 | MCMPCFTTDTQMQERCDRCCGGGGRGRCWGPQCLCI |
| SEQ ID NO: 101 | MCMPCFTTEQRMAIICDDCCGGFGRGRCYGPQCLCR |
| SEQ ID NO: 102 | ICIPCFTTDHQIARRCDDCCGGRGRGRCYGPQCICR |
| SEQ ID NO: 103 | RCQLQGFNCVVRSYGLPTIPCCRGLTCRSYFPGSTYGRCQRY |
| SEQ ID NO: 104 | SFGLCRLRRGFCARGRCRFPSIPIGRCSRFVQCCRRVW |
| SEQ ID NO: 105 | SCEPGTTFRDRCNTCRCGSDGRSAACTLRACPQ |
| SEQ ID NO: 106 | SCTPGTTFRDRCNTCRCSSNGRSAACTLRACPPGSY |
| SEQ ID NO: 107 | SCTPGTTFRNRCNTCRCGSNGRSASCTLMACPPGSY |
| SEQ ID NO: 108 | SCTPGATFRNRCNTCRCGSNGRSASCTLMACPPGSY |
| SEQ ID NO: 109 | SCQPGTTYQRGCNTCRCLEDGQTEACTLRLC |
| SEQ ID NO: 110 | SCTPGATYREGCNICRCRSDGRSGACTRRICPVDSN |
| SEQ ID NO: 111 | SCQPGTTFRRDCNTCVCNRDGTNAACTLRACL |
| SEQ ID NO: 112 | YSRCQLQGFNCVVRSYGLPTIPCCRGLTCRSYFPGSTYGRCQRY |
| SEQ ID NO: 113 | SCARPRENCNRMNILCCRGECVCPTFGDCFCYGD |
| SEQ ID NO: 114 | GVPINVRCRGSRDCLDPCRRAGMRFGRCINSRCHCTP |
| SEQ ID NO: 115 | SERDCIRHLQRCRENRDCCSRRCSRRGTNPERRCR |
| SEQ ID NO: 116 | VRIPVSCRHSGQCLRPCRDAGMRFGRCMNGRCDCTPR |
| SEQ ID NO: 117 | QVQTNVRCQGGSCASVCRREIGVAAGRCINGRCVCYRN |
| SEQ ID NO: 118 | GDCLPHLRRCRENNDCCSRRCRRRGANPERRCR |
| SEQ ID NO: 122 | GGDCLPHLRRCRADNDCCGRRCRRRGTNAERRCR |
| SEQ ID NO: 123 | GGDCKYKFENWGACDGGTGTKVRQGTLKKARYNAQCQETIRVTKPC |
| SEQ ID NO: 124 | GGDCKYKFENWGACDGGTGTKVRQGTLKKARYNAQCQETIRVTKPCT |
| | PKTKAKAKAKKGKGKD |
| SEQ ID NO: 125 | GGSCEPGRTFRDRCNTCRCGADGRSAACTLRACPNQ |
| SEQ ID NO: 126 | GGQFTNVSCITSRECWSVCQRLHNTSRGRCMNRRCRCYS |
| SEQ ID NO: 127 | GGMCMPCFTTDHQMARRCDDCCGGRGRGRCYGPQCLCR |
| SEQ ID NO: 128 | GGISIGIKCSPSIDLCEGQCRIRKYFTGYCSGDTCHCSG |
| SEQ ID NO: 129 | GGEVIRCSGSKQCYGPCKQQTGCTNSKCMNKVCKCYGCG |
| SEQ ID NO: 130 | GGSEKDCIKHLQRCRENKDCCSKKCSRRGTNPEKRCR |
| SEQ ID NO: 131 | GGSCAKPRENCNRMNILCCRGECVCPTFGDCFCYGD |
| SEQ ID NO: 132 | GGGVPINVKCRGSRDCLDPCKKAGMRFGKCINSKCHCTP |
| SEQ ID NO: 133 | GGVRIPVSCKHSGQCLKPCKDAGMRFGKCMNGKCDCTPK |
| SEQ ID NO: 134 | GGGIVCKVCKIICGMQGKKVNICKApIKCKCKKG |
| SEQ ID NO: 135 | GGDCVREWGKCSQTSDCCPHLACKSKWPRNICVWDGSVG |
| SEQ ID NO: 136 | GGAVCVYRTCDKDCKRRGYRSGKCINNACKCYPYG |
| SEQ ID NO: 137 | GGGCFGYKCDYYKGCCSGYVCSPTWKWCVRPGPGR |
| SEQ ID NO: 138 | GGQVQTNVKCQGGSCASVCRREIGVAAGKCINGKCVCYRN |
| SEQ ID NO: 139 | GGGDCLPHLKRCKENNDCCSKKCKRRGANPEKRCR |
| SEQ ID NO: 140 | GGNFKVEGACSKPCRKYCIDKGARNGKCINGRCHCYY |
| SEQ ID NO: 141 | GGQKILSNRCNNSSECIPHCIRIFGTRAAKCINRKCYCYP |
| SEQ ID NO: 142 | GGDRDSCIDKSRCSKYGYYQECQDCCKKAGHNGGTCMFFKCKCA |
| SEQ ID NO: 143 | GGAVCNLKRCQLSCRSLGLLGKCIGDKCECVKHG |
| SEQ ID NO: 144 | GGQFCGTNGKPCVNGQCCGALRCVVTYHYADGVCLKMNP |
| SEQ ID NO: 145 | GGRPTDIKCSASYQCFPVCKSRFGKTNGRCVNGLCDCF |
| SEQ ID NO: 146 | GGNCAGYMRECKEKLCCSGYVCSSRWKWCVLPAPWRR |
| SEQ ID NO: 147 | GGQFTDVKCTGSKQCWPVCKQMFGKPNGKCMNGKCRCYS |
| SEQ ID NO: 148 | GGQIDTNVKCSGSSKCVKICIDRYNTRGAKCINGRCTCYP |
| SEQ ID NO: 149 | GGAEIIRCSGTRECYAPCQKLTGCLNAKCMNKACKCYGCV |
| SEQ ID NO: 150 | GGSDYCSNDFCFFSCRRDRCARGDCENGKCVCKNCHLN |
| SEQ ID NO: 151 | GGCIGEGVPCDENDPRCCFGLVCLKPTLHGIWYKSYYCYKK |
| SEQ ID NO: 152 | GGSCAKPGEMCMRIKCCDGQCGCNRGTGRCFCK |
| SEQ ID NO: 153 | GGACLAEYQKCEGSTVPCCPGLSCSAGRFRKTKLCTK |
| SEQ ID NO: 154 | GGVVIGQRCYRSPDCYSACKKLVGKATGKCTNGRCDC |
| SEQ ID NO: 155 | GGACQFWSCNSSCISRGYRQGYCWGIQYKYCQCQ |
| SEQ ID NO: 156 | GGRCPPCFTTNPNMEADCRKCCGGRGYCASYQCICPGG |
| SEQ ID NO: 157 | GGVFINVKCRGSPECLPKCKEAIGKSAGKCMNGKCKCYP |
| SEQ ID NO: 158 | GGQVSTNKKCSNTSQCYKTCEKVVGVAAGKCMNGKCICYP |
| SEQ ID NO: 159 | GGECLEIFKACNPSNDQCCKSSKLVCSRKTRWCKYQIG |
| SEQ ID NO: 160 | GGQDKCKKVYENYPVSKCQLANQCNYDCKLDKHARSGECFYDEKRNL |
| | QCICDYCEY |
| SEQ ID NO: 161 | GGGHACYRNCWREGNDEETCKERC |
| SEQ ID NO: 162 | GGMCMPCFTTDTQMQERCDRCCGGGGRGRCWGPQCLCI |
| SEQ ID NO: 163 | GGMCMPCFTTEQRMAIICDDCCGGFGRGRCYGPQCLCR |
| SEQ ID NO: 164 | GGICIPCFTTDHQIARRCDDCCGGRGRGRCYGPQCICR |
| SEQ ID NO: 165 | GGRCQLQGFNCVVRSYGLPTIPCCRGLTCRSYFPGSTYGRCQRY |
| SEQ ID NO: 166 | GGSFGLCRLRRGFCARGRCRFPSIPIGRCSRFVQCCRRVW |
| SEQ ID NO: 167 | GGSCEPGTTFRDRCNTCRCGSDGRSAACTLRACPQ |
| SEQ ID NO: 168 | GGSCTPGTTFRDRCNTCRCSSNGRSAACTLRACPPGSY |
| SEQ ID NO: 169 | GGSCTPGTTFRNRCNTCRCGSNGRSASCTLMACPPGSY |
| SEQ ID NO: 170 | GGSCTPGATFRNRCNTCRCGSNGRSASCTLMACPPGSY |
| SEQ ID NO: 171 | GGSCQPGTTYQRGCNTCRCLEDGQTEACTLRLC |
| SEQ ID NO: 172 | GGSCTPGATYREGCNICRCRSDGRSGACTRRICPVDSN |
| SEQ ID NO: 173 | GGSCQPGTTFRRDCNTCVCNRDGTNAACTLRACL |
| SEQ ID NO: 174 | GSYSRCQLQGFNCVVRSYGLPTIPCCRGLTCRSYFPGSTYGRCQRY |
| SEQ ID NO: 175 | GGSCARPRENCNRMNILCCRGECVCPTFGDCFCYGD |
| SEQ ID NO: 176 | GGGVPINVRCRGSRDCLDPCRRAGMRFGRCINSRCHCTP |
| SEQ ID NO: 177 | GGSERDCIRHLQRCRENRDCCSRRCSRRGTNPERRCR |
| SEQ ID NO: 178 | GGVRIPVSCRHSGQCLRPCRDAGMRFGRCMNGRCDCTPR |
| SEQ ID NO: 179 | GGQVQTNVRCQGGSCASVCRREIGVAAGRCINGRCVCYRN |
| SEQ ID NO: 180 | GGGDCLPHLRRCRENNDCCSRRCRRRGANPERRCR |

Identifying sequence homology can be important for determining key residues that preserve kidney targeting function. For example, conservation of hydrophilic residues, such as N, Q, S, T, D, E, K, R, and H, can be important for preserving peptide kidney targeting function by keeping the peptide from sticking to albumin. Additionally, basic amino acids such as Lys and/or Arg can important to binding and retention of a peptide in the kidney. Two or more peptides can share a degree of homology and share similar properties in vivo. For instance, a peptide of the present disclosure can share a degree of homology with a peptide of any of SEQ ID NO: 1-SEQ ID NO: 118, or a fragment thereof. In some cases, a peptide of the disclosure can have up to about 20% pairwise homology, up to about 25% pairwise homology, up to about 30% pairwise homology, up to about 35% pairwise homology, up to about 40% pairwise homology, up to about 45% pairwise homology, up to about 50% pairwise homology, up to about 55% pairwise homology, up to about 60% pairwise homology, up to about 65% pairwise homology, up to about 70% pairwise homology, up to about 75% pairwise homology, up to about 80% pairwise homology, up to about 85% pairwise homology, up to about 90% pairwise homology, up to about 95% pairwise homology, up to about 96% pairwise homology, up to about 97% pairwise homology, up to about 98% pairwise homology, up to about 99% pairwise homology, up to about 99.5% pairwise homology, or up to about 99.9% pairwise homology with a second peptide. In some cases, a peptide of the disclosure can have at least about 20% pairwise homology, at least about 25% pairwise homology, at least about 30% pairwise homology, at least about 35% pairwise homology, at least about 40% pairwise homology, at least about 45% pairwise homology, at least about 50% pairwise homology, at least about 55% pairwise homology, at least about 60% pairwise homology, at least about 65% pairwise homology, at least about 70% pairwise homology, at least about 75% pairwise homology, at least about 80% pairwise homology, at least about 85% pairwise homology, at least about 90% pairwise homology, at least about 95% pairwise homology, at least about 96% pairwise homology, at least about 97% pairwise homology, at least about 98% pairwise homology, at least about 99% pairwise homology, at least about 99.5% pairwise homology, at least about 99.9% pairwise homology with a second peptide. Various methods and software programs can be used to determine the homology between two or more peptides, such as NCBI BLAST, Clustal W, MAFFT, Clustal Omega, AlignMe, Praline, or another suitable method or algorithm.

In still other instances, the variant nucleic acid molecules of a peptide of any one of SEQ ID NO: 1-SEQ ID NO: 118 can be identified by either a determination of the sequence identity or homology of the encoded peptide amino acid sequence with the amino acid sequence of any one of SEQ ID NO: 1-SEQ ID NO: 118, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 118 (or any complement of the previous sequences) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity or homology to the amino acid sequence of any one SEQ ID NO: 1-SEQ ID NO: 118. Alternatively, peptide variants of any one SEQ ID NO: 1-SEQ ID NO: 118 can be characterized as nucleic acid molecules (1) that remain hybridized with a nucleic acid molecule having the nucleotide sequence of any one SEQ ID NO: 1-SEQ ID NO: 118 (or any complement of the previous sequences) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 118.

Percent sequence identity or homology can be determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol.183:63 (1990).

FASTA can also be used to determine the sequence identity or homology of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that can be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2, or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments determination of structure can typically be accompanied by evaluating activity of modified molecules.

Pairwise sequence alignment is used to identify regions of similarity that can indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

In some embodiments, the first two N-terminal amino acids of SEQ ID NO: 1-SEQ ID NO: 59 (GS for SEQ ID NO: 1-SEQ ID NO: 52 and SEQ ID NO: 54-SEQ ID NO: 59, GG for SEQ ID NO: 53) serve as a spacer or linker in order to facilitate conjugation or fusion to another molecule, as well as to facilitate cleavage of the peptide from such conjugated or fused molecules. In some embodiments, the peptide may or may not include the first two N-terminal amino acids shown in SEQ ID NO: 1-SEQ ID NO: 59, or such N-terminal amino acids can be substituted by any other one or two amino acids, as shown in SEQ ID NO: 60-SEQ ID NO: 118. For example, in certain embodiments, the first two N-terminal amino acids (GS) of SEQ ID NO: 1-SEQ ID NO: 52 and SEQ ID NO: 54-SEQ ID NO: 59 are substituted with GG as in SEQ ID NO: 122-SEQ ID NO: 173 and SEQ ID NO: 175-SEQ ID NO: 180. As another example, in certain embodiments, the first two N-terminal amino acids (GG) of SEQ ID NO: 53 are substituted with GS as in SEQ ID NO: 174.

In some embodiments, the peptide sequence is flanked by additional amino acids. One or more additional amino acids can, for example, confer a desired charge under physiological conditions, isoelectric point, chemical conjugation site, stability, or physiologic property to a peptide. For instance, the amine in a lysine residue or the N-terminus can serve as a chemical conjugation site. Other lysine residues can be mutated out, such as by substitution with arginine, to provide a single site for amine conjugation.

The present disclosure encompasses various modifications to the peptides provided herein. In some embodiments, a peptide of the present disclosure contains or is modified to contain only one lysine residue, or no lysine residues. In some embodiments, some or all of the lysine residues in the peptide are replaced with arginine residues. In some embodiments, some or all of the methionine residues in the peptide are replaced by leucine or isoleucine. In some embodiments, some or all of the tryptophan residues in the peptide are replaced by phenylalanine or tyrosine. In some embodiments, some or all of the asparagine residues in the peptide are replaced by glutamine. In some embodiments, some or all of the cysteine residues in the peptide are replaced by serine to produce a linearized form of the peptide. In some embodiments, the N-terminus of the peptide is blocked, such as by an acetyl group. In some embodiments, the N-terminus of the peptide is blocked with pyroglutamic acid. Alternatively or in combination, in some instances, the C-terminus of the peptide is blocked, such as by an amide group. In some embodiments, the peptide is modified by methylation on free amines. For example, full methylation can be accomplished through the use of reductive methylation with formaldehyde and sodium cyanoborohydride.

At physiological pH, peptides can have a net charge, for example, of −5, −4, −3, −2, −1, 0, +1, +2, +3, +4, +5, +6, +7, +8, +9, or +10. When the net charge is zero, the peptide can be uncharged or zwitterionic. In some embodiments, the engineering of one or more mutations within a peptide yields a peptide with an altered isoelectric point, charge, surface charge, or rheology at physiological pH. Such engineering of a mutation to a peptide of the present disclosure (e.g., a peptide derived from a scorpion or spider) can change the net charge of the complex, for example, by decreasing the net charge by 1, 2, 3, 4, or 5, or by increasing the net charge by 1, 2, 3, 4, or 5.

In certain embodiments, the engineered mutation can facilitate the ability of the peptide to bind to renal tissue. Suitable amino acid modifications for improving the rheology and potency of a peptide can include conservative or non-conservative mutations. A peptide can comprise at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the peptide scaffold (e.g., venom or toxin component) that the peptide is derived from. In other cases, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the peptide scaffold (e.g., venom or toxin component) that the peptide is derived from. In some embodiments, mutations can be engineered within a peptide to provide a peptide that has a desired charge or stability at physiological pH.

In some embodiments, the peptide can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to modify, add or delete binding behavior in vivo, add new targeting function, modify surface charge and hydrophobicity, or allow conjugation sites.

In some embodiments, more than one peptide sequence is present on a particular peptide. For example, a peptide of the present disclosure can include sequences from at least 1, at least 2, at least 3, at least 4, at least 5, at least 6, at least 7, at least 8, at least 9, or at least 10 different peptides, or fragments thereof.

In some embodiments, the peptide described herein can be attached to another molecule. For example, the peptide sequence also can be attached to another active agent (e.g., small molecule, peptide, polypeptide, polynucleotide, antibody, antibody fragment, single chain Fv, aptamer, cytokine, growth factor, neurotransmitter, an active fragment or modification of any of the preceding, fluorophore, radioisotope, radionuclide chelator, acyl adduct, chemical linker, or sugar, etc.). In some embodiments, the peptide can be fused with, or covalently or non-covalently linked to an active agent.

In some embodiments, a peptide of the present disclosure is incorporated into a biomolecule, e.g., a protein. A peptide can be incorporated into a biomolecule by various techniques. A peptide can be incorporated by a chemical transformation, such as the formation of a covalent bond (e.g., an amide bond). A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

The present disclosure also encompasses multimers of the various peptides described herein. Examples of multimers include dimers, trimers, tetramers, pentamers, hexamers, heptamers, and so on. A multimer can be a homomer formed from a plurality of identical subunits or a heteromer formed from a plurality of different subunits. In some embodiments, a peptide of the present disclosure is arranged in a multimeric structure with at least one other peptide, e.g., two, three, four, five, six, seven, eight, nine, ten, or more other peptides. In certain embodiments, the peptides of a multimeric structure each have the same sequence. In alternative embodiments, some or all of the peptides of a multimeric structure have different sequences.

### Peptide Properties for Renal Localization, Binding and Internalization

The present disclosure provides peptides that can distribute to, home, target, be directed to, accumulate in, migrate to, be retained in, and/or bind to one or more specific regions, tissue, structures, regions, compartments, or cells of the kidney, collectively referred to herein as “renal tissue.” Examples of regions, tissue, structures, or cells of the kidney applicable to the embodiments presented herein include but are not limited to: the cortex region, the glomerulus, the glomerular filtrate (Bowman's space) tubular lumina, the proximal tubule, the S1, S2, and S3 segments, the medulla region, the descending tubule, the ascending tubule, the distal tubule, the loop of Henle, the Bowman's capsule, the renal interstitium, the renal microvasculature, vasa rectae, or any cells or cell types thereof.

In some embodiments, the peptides of the present disclosure interact with renal tissue of the subject, e.g., by binding to the renal tissue. The binding between the peptide and the renal tissue can be a specific binding interaction (e.g., a receptor-ligand interaction) or non-specific binding interaction (e.g., electrostatic interaction). For example, in certain embodiments, upon administration to a subject, a peptide of the present disclosure binds to a proximal tubule of the subject, e.g., a cell of the proximal tubule. As another example, in certain embodiments, upon administration to a subject, a peptide of the present disclosure binds to a glomerulus of the subject, e.g., a cell of the glomerulus. As another example, in certain embodiments, a peptide of the present disclosure binds to podocytes. In various embodiments, the peptides bind to receptors expressed by a renal cell. For instance, a peptide can bind to a cell surface receptor expressed by a cell of the proximal tubule, a megalin receptor, a cubulin receptor, or a combination thereof.

In some embodiments, the peptides are internalized by a cell of the renal tissue of the subject. The present disclosure encompasses various types of internalization mechanisms, including but not limited to pinocytosis, phagocytosis, endocytosis, receptor-mediated endocytosis, scavenging mechanisms, membrane penetration or translocation mechanisms, or combinations thereof. For example, a peptide can be internalized following binding to the cell or a receptor thereof, e.g., via receptor-mediated endocytosis.

Certain embodiments of the peptides described herein exhibit properties that enhance localization, binding, accumulation in, and/or internalization by renal tissues, regions, compartments, or cells. Examples of peptide properties that can be relevant to renal binding and internalization include but are not limited to isoelectric point, net charge, charge distribution, molecular weight, hydrodynamic radius, pH stability, hydrophilicity, and protein-protein binding.

For example, in various embodiments, the peptides of the present disclosure exhibit an isoelectric point (pI) favorable for renal localization, binding, and/or internalization. In certain embodiments, the pI of a peptide is less than or equal to about 2.0, less than or equal to about 2.5, less than or equal to about 3.0, less than or equal to about 3.5, 4.0, less than or equal to about 4.5, less than or equal to about 5.5, less than or equal to about 6.0, less than or equal to about 6.5, less than or equal to about 7.0, less than or equal to about 7.5, less than or equal to about 8.0, less than or equal to about 8.5, less than or equal to about 9.0, less than or equal to about 9.5, less than or equal to about 10.0, less than or equal to about 10.5, less than or equal to about 11.0, less than or equal to about 11.5, less than or equal to about 12.0, less than or equal to about 12.5, less than or equal to about 13.0, less than or equal to about 13.5, less than or equal to about 14.0, less than or equal to about 14.5, or less than or equal to about 15.0. In certain embodiments, the pI of a peptide is greater than or equal to about 2.0, greater than or equal to about 2.5, greater than or equal to about 3.0, greater than or equal to about 3.5, 4.0, greater than or equal to about 4.5, greater than or equal to about 5.5, greater than or equal to about 6.0, greater than or equal to about 6.5, greater than or equal to about 7.0, greater than or equal to about 7.5, greater than or equal to about 8.0, greater than or equal to about 8.5, greater than or equal to about 9.0, greater than or equal to about 9.5, or greater than or equal to about 10.0, greater than or equal to about 10.5, greater than or equal to about 11.0, greater than or equal to about 11.5, greater than or equal to about 12.0, greater than or equal to about 12.5, greater than or equal to about 13.0, greater than or equal to about 13.5, greater than or equal to about 14.0, greater than or equal to about 14.5, or greater than or equal to about 15.0. The pI of a peptide can be within a range from about 3.0 to about 10.0, within a range from about 3.0 to about 6.0, or within a range from about 4.0 to about 9.0.

In some embodiments, the pI (the pH at which the net charge of the peptide is zero) of the peptides of this disclosure can be calculated by the EMBOSS method. The pI value is the isoelectric point of fully reduced form of protein sequences. The value can be calculated with the Henderson-Hasselbalch equation using EMBOSS scripts and a pKa table provided by the European Bioinformatics Institute. The EMBOSS method of calculating pI has been described by Rice et al. (EMBOSS: the European Molecular Biology Open Software Suite. Trends Genet. 2000 June; 16(6):276-7) and Carver et al. (The design of Jemboss: a graphical user interface to EMBOSS. Bioinformatics. 2003 Sep. 22; 19(14):1837-43). In some embodiments, peptides of the present disclosure with a pI value greater than 9 can have higher accumulation in the kidneys.

In some embodiments, the pI of the peptide influences its localization within the kidney. For example, in certain embodiments, higher pI values (e.g., greater than or equal to about 7.5) promote localization and/or binding to the glomerulus, while lower pI values (e.g., lower than 7.5) promote localization and/or binding to the proximal tubule. Accordingly, different localization patterns within the kidney can be achieved by varying the pI of the peptide. In certain embodiments, the osmotic concentration of the urine and/or urine flow rates have an impact on intratubular localization.

As another example, in various embodiments, the peptides of the present disclosure exhibit a charge distribution at neutral pH favorable for renal localization, binding, and/or internalization. In certain embodiments, the peptide exhibits a substantially uniform charge distribution. In alternative embodiments, the peptide exhibits a non-uniform charge distribution, e.g., including one or more regions of concentrated positive charge and/or one or more regions of concentrated negative charge. The charge distribution can impact the localization, binding and/or internalization of the peptide. For example, the glomerular capillary wall and/or slit processes are negatively charged, which in certain embodiments influences glomerular localization of middle sized positively charged molecules (e.g., having a mass-average molecular weight (Mw) within a range from about 30 kDa to about 60 kDa), while being less likely to influence localization of smaller molecules (e.g., having a Mw less than 30 kDa) such as knotted peptides. In certain embodiments, the charge distribution of the peptide influences electrostatic interactions with a target, e.g., the megalin/cubulin receptor.

In yet another example, in various embodiments, the peptides of the present disclosure exhibit a molecular weight favorable for renal targeting, localization, binding, accumulation, and/or internalization. In certain embodiments, the peptide comprises a mass-average molecular weight (Mw) less than or equal to about 1 kDa, less than or equal to about 2 kDa, less than or equal to about 3 kDa, less than or equal to about 4 kDa, less than or equal to about 5 kDa, less than or equal to about 6 kDaor less than or equal to about 10 kDa, less than or equal to about 20 kDa, less than or equal to about 30 kDa, less than or equal to about 40 kDa, less than or equal to about 50 kDa, less than or equal to about 60 kDa, or less than or equal to about 70 kDa. In certain embodiments, the peptide comprises a Mw within a range from about 0.5 kDa to about 50 kDa, or within a range from about 0.5 kDa to about 60 kDa.

In some embodiments, molecules (e.g., proteins or peptides) having relatively low Mw (e.g., less than or equal to about 1 kDa, less than or equal to about 2 kDa, less than or equal to about 3 kDa, less than or equal to about 4 kDa, less than or equal to about 5 kDa, less than or equal to about 10 kDa, less than or equal to about 20 kDa, less than or equal to about 30 kDa, or less than or equal to about 60 kDa) are rapidly targeted to, localized, bound, accumulated, and/or internalized by the kidney. In certain embodiments, low Mw molecules are freely filtered, presented to the proximal tubules of the kidney, and optionally taken up by megalin/cubulin receptors. In certain embodiments, low molecular weight molecules undergo endocytic reabsorption via the megalin/cubulin pathway and are then trafficked to renal tubular lysosomes for processing. In some embodiments, molecules (e.g., proteins or peptides) having higher Mw (e.g., greater than about 70 kDa) are generally excluded from glomerular filtration, but can still be able to achieve interstitial localization via the microcirculation.

In a further example, in various embodiments, the peptides of the present disclosure exhibit stability at pH values favorable for renal localization, binding, and/or internalization. A peptide can be considered to be stable at a certain pH if it is capable of performing its functional or therapeutic effect, is soluble, is resistant to protease degradation, is resistant to reduction, retains secondary or tertiary structure, or a combination thereof. In certain embodiments, the peptide is stable at pH values less than or equal to about 3.0, less than or equal to about 3.5, 4.0, less than or equal to about 4.5, less than or equal to about 5.5, less than or equal to about 6.0, less than or equal to about 6.5, less than or equal to about 7.0, less than or equal to about 7.5, less than or equal to about 8.0, less than or equal to about 8.5, less than or equal to about 9.0, less than or equal to about 9.5, or less than or equal to about 10.0. In certain embodiments, the peptide is stable at pH values greater than or equal to about 3.0, greater than or equal to about 3.5, 4.0, greater than or equal to about 4.5, greater than or equal to about 5.5, greater than or equal to about 6.0, greater than or equal to about 6.5, greater than or equal to about 7.0, greater than or equal to about 7.5, greater than or equal to about 8.0, greater than or equal to about 8.5, greater than or equal to about 9.0, greater than or equal to about 9.5, or greater than or equal to about 10.0. In certain embodiments, the peptide is stable at pH values within a range from about 3.0 to about 5.0, and/or within a range from about 5.0 to about 7.0.

As previously discussed, in some embodiments, the disulfide knot structure of knotted peptides confers improved stability over a wide range of pH values, which can be advantageous for renal applications. For example, stability at low pH values can be advantageous in order to avoid cast formation leading to intratubular obstruction. In some embodiments, cast formation occurs via co-precipitation of proteins with an endogenously produced glycoprotein known as Tamm Horsall protein. In certain embodiments, this precipitation is affected by urinary pH and osmolality, as precipitation typically occurs under acidic conditions (e.g., pH less than about 5) and high salt concentrations and/or osmolality. Alternatively or in combination, stability at low pH value can reduce or prevent lysosomal degradation, which can improve delivery precision and avoid broader cellular or systemic toxicity.

### Chemical Modifications and Conjugates of Peptides

A peptide can be chemically modified one or more of a variety of ways. For example, N-methylation is one example of methylation that can occur in a peptide of the disclosure. A chemical modification can, for instance, change the biodistribution or pharmacokinetic profile. A chemical modification can comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region can be a fusion Fc-peptide. A polyamino acid can include, for example, a polyamino acid sequence with repeated single amino acids (e.g., polyglycine), and a polyamino acid sequence with mixed polyamino acid sequences (e.g., gly-ala-gly-ala) that may or may not follow a pattern, or any combination of the foregoing.

Peptides according to the present disclosure can be conjugated or fused to an agent for use in the treatment of renal diseases, disorders, or injuries. For example, in certain embodiments, a peptide as described herein can be fused to another molecule, such as an active agent that provides a functional capability. The active agent can function as a renal therapeutic agent, a renal protective agent, or renal prophylactic agent. A peptide can be fused with an active agent through expression of a vector containing the sequence of the peptide with the sequence of the active agent. In various embodiments, the sequence of the peptide and the sequence of the active agent are expressed from the same Open Reading Frame (ORF). In various embodiments, the sequence of the peptide and the sequence of the active agent can comprise a contiguous sequence. The peptide and the active agent can each retain similar functional capabilities in the fusion peptide compared with their functional capabilities when expressed separately.

Furthermore, for example, in certain embodiments, the peptides described herein are attached to another molecule, such as an active agent that provides a functional capability.

In some embodiments, 1, 2, 3, 4, 5, 6, 7, 8, 9, or 10 active agents can be linked to a peptide. Multiple active agents can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, a single chain variable fragment (scFv, or a single chain Fv), an antibody fragment, an aptamer, a cytokine, an interferon, a hormone, an enzyme, a growth factor, a checkpoint inhibitor, a PD-1 inhibitor, a PD-L1 inhibitor, a CTLA4 inhibitor, a CD antigen, aa chemokine, a neurotransmitter, an ion channel inhibitor, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a tumor necrosis factor (TNF) soluble receptor or antibody, caspase protease activator or inhibitor, an NF-κB a RIPK1 and/or RIPK3 inhibitor or activator (e.g., through Toll-like receptors (TLRs) TLR-3 and/or TLR-4, or T-cell receptor (TCR) and the like), a death-receptor ligand (e.g., Fas ligand) activator or inhibitor, TNF receptor family (e.g., TNFR1, TNFR2, lymphotoxin f3 receptor/TNFRS3, OX40/TNFRSF4, CD40/TNFRSF5, Fas/TNFRSF6, decoy receptor 3/TNFRSF6B, CD27/TNFRSF7, CD30/TNFRSF8, 4-1BB/TNFRSF9, DR4 (death receptor 4/TNFRS10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TAC1/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17), GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27, ectodysplasin 1, and anhidrotic receptor, a TNF receptor superfamily ligand including—TNF alpha, lymphotoxin-α, tumor necrosis factor membrane form, tumor necrosis factor shed form, LIGHT, lymphotoxin β₂α₁ heterotrimer, OX-40 ligand, compound 1 [PMID: 24930776], CD40 ligand, Fas ligand, TL1A, CD70, CD30 ligand, TRAF1, TRAF2, TRAF3, TRAIL, RANK ligand, APRIL, BAFF, B and T lymphocyte attenuator, NGF, BDNF, neurotrophin-3, neurotrophin-4, TL6, ectodysplasin A2, ectodysplasin A1—a TIMP-3 inhibitor, a BCL-2 family inhibitor, an IAP disruptor, a protease inhibitor, an amino sugar, a chemotherapeutic (whether acting through an apoptotic or non-apoptotic pathway) (Ricci et al. Oncologist 11(4):342-57 (2006)), a cytotoxic chemical, a toxin, a tyrosine kinase inhibitor (e.g. imatinib mesylate), QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO and EPO derivatives, agents that stimulate erthyropoietin such as epoeitn alfa or darbepoietin alfa, PDGF inhibitors, CRMD-001, Atrasentan, Tolvaptan, RWJ-676070, Abatacept, Sotatercept, the binding site of the extracellular domain of the activing receptor 2A, an anti-infective agent, an antibiotic such as gentamicin, vancomycin, minocin or mitomyclin, an anti-viral agent, an anti-fungal agent, an aminoglycoside, a nonsteroidal anti-inflammatory drug (NSAID) such as ketorolac or ibuprofen, an immunosuppresant such tacrolimus, mycophenolic acid (e.g., mycophenolate mofetil), cyclosporine A, or azathioprine, a diuretic drug such as thiazides, bemetanide, ethacrynic acid, furosemidem torsemide, glucose, mannitol, amiloride, spironolactone, eplerenone, triamterene, potassium canrenoate, bendroflumethiazide, hydrochlorothiazide, vasopressin, amphotericin B, acetazolamide, tovaptan, conivaptan, dopamine, dorzolamide, bendrolumethiazide, hydrochlorothiazide, caffeine, theophylline, or theobromine, a statin, a senolytic such as navitoclax or obatoclax, a corticosteroid such as prednisone, betamethasone, fludrocortisone, deoxycorticosterone, aldosterone, cortisone, hydrocortisone, belcometasone, dexamethasone, mometasone, fluticasone, prednisolone, methylprednisolone, triamcinolone acetonide or triamcinolone, a glucocorticoid, a liposome, renin, angiotensin, ACE inhibitors such as ramipril, captopril, lisinopril, benazepril, quinapril, fosinopril, trandolapril, moexipril, enalaprilat, enalapril maleate, or perindopril erbumine, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, retinoic acid, SGLT2 modulator, a polymer, a biopolymer, a polysaccharide, a proteoglycan, a glycosaminoglycan, polyethylene glycol, a lipid, a dendrimer, a fatty acid, or an Fc domain or an Fc region, or an active fragment or a modification thereof.

Any combination of the above active agents can be co-delivered with peptides or peptide conjugates of this disclosure. Additionally, in some embodiments, other co-therapies such as proton therapy or ablative radiotherapy can be administered to a subject in need thereof along with peptides or peptide conjugates of this disclosure. In some embodiments, the peptide is covalently or non-covalently linked to an active agent, e.g., directly or via a linker. TNF blockers suppress the immune system by blocking the activity of TNF, a substance in the body that can cause inflammation and lead to immune-system diseases, such as Crohn's disease, ulcerative colitis, rheumatoid arthritis, ankylosing spondylitis, psoriatic arthritis and plaque psoriasis. The peptide disclosed herein can be used to home, distribute to, target, directed to, is retained by, accumulate in, migrate to, and/or bind to the kidneys, and thus also be used for localizing the attached or fused active agent. Furthermore, knotted chlorotoxin peptide can be internalized in cells (Wiranowska, M., Cancer Cell Int., 11: 27 (2011)). Therefore, cellular internalization, subcellular localization, and intracellular trafficking after internalization of the active agent peptide conjugate or fusion peptide can be important factors in the efficacy of an active agent conjugate or fusion. (Ducry, L., Antibody Drug Conjugates (2013); and Singh, S. K., Pharm Res. 32(11): 3541-3571 (2015)).

In some embodiments, the peptides of the present disclosure are coupled (e.g., conjugated) to other moieties that, e.g., can modify or effect changes to the properties of the peptides. For example, in certain embodiments, the peptides described herein are attached to another molecule, such as an active agent that provides a functional capability. Examples of active agents include but are not limited to: a peptide, an oligopeptide, a polypeptide, a polynucleotide, a polyribonucleotide, a DNA, a cDNA, a ssDNA, a RNA, a dsRNA, a micro RNA, an oligonucleotide, an antibody fragment, a single chain Fv, an aptamer, a cytokine, an enzyme, a growth factor, a chemokine, a neurotransmitter, a chemical agent, a fluorophore, a metal, a metal chelate, an X-ray contrast agent, a PET agent, a radioisotope, a photosensitizer, a radiosensitizer, a radionuclide chelator, a therapeutic small molecule, a steroid, a corticosteroid, an anti-inflammatory agent, an immune modulator, a protease inhibitor, an amino sugar, a chemotherapeutic, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID) such as ketorolac or ibuprofen, a statin, a nanoparticle, a liposome, a polymer, a biopolymer, a polysaccharide, a proteoglycan, a glycosaminoglycan, a dendrimer, a fatty acid, or an Fc region, or an active fragment or a modification thereof. In some embodiments, the peptide is covalently or non-covalently linked to an active agent, e.g., directly or via a linker. Exemplary linkers suitable for use with the embodiments herein are discussed in further detail below.

Optionally, certain embodiments of the present disclosure provide peptides conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide is covalently or non-covalently linked to the agent, e.g., directly or via a linker. Exemplary linkers suitable for use with the embodiments herein are discussed in further detail below.

In some embodiments, the active agent interacts with a renal ion channel, inhibits a protease, has antimicrobial activity, has anticancer activity, has anti-inflammatory activity, induces ischemic preconditioning or acquired cytoresistance, produces a protective or therapeutic effect on a kidney of the subject, reduces a clearance rate of the composition, or a combination thereof. Optionally, the active agent is a renal therapeutic agent, such as a renal protective agent or renal prophylactic agent that induces ischemic preconditioning and/or acquired cytoresistance in a kidney of a subject. Additional details regarding renal therapeutic agents are provided below.

In some embodiments, the peptides of the present disclosure can be modified such that the modification increases the stability and/or the half-life of the peptides. In some embodiments, the attachment of a hydrophobic moiety, such as to the N-terminus, the C-terminus, or on an internal amino acid, can be used to extend half-life of a peptide of the present disclosure. In some embodiments, simple carbon chains (e.g., by myristoylation and/or palmitylation) can be conjugated to the fusion proteins or peptides. In some embodiments, the simple carbon chains can render the peptides easily separable from the unconjugated material. For example, methods that can be used to separate the peptides from the unconjugated material include, but are not limited to, solvent extraction and reverse phase chromatography. The lipophilic moieties can extend half-life through reversible binding to serum albumin. The conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof.

Other modifications to the peptides or of the present disclosure can be used. For example, the peptides of the present disclosure can include post-translational modifications (e.g., methylation and/or amidation), which can affect, e.g., serum half-life. In some embodiments, the can be conjugated to other moieties that, e.g., can modify or effect changes to the properties of the peptides. The conjugated moieties can, e.g., be lipophilic moieties that extend half-life of the peptides through reversible binding to serum albumin. In some embodiments, simple carbon chains (e.g., by myristoylation) can be conjugated to the peptides. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof.

In some embodiments, the peptides of the present disclosure are coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. The linker can be cleavable or noncleavable.

A peptide can be conjugated to an agent used in imaging, research, therapeutics, theranostics, pharmaceuticals, chemotherapy, chelation therapy, targeted drug delivery, and radiotherapy. In some embodiments, the peptides of the present disclosure are coupled (e.g., conjugated) to a detectable agent using any of the linkers described herein and any conjugation method described herein. Examples of detectable agents include metals, radioisotopes, dyes, fluorophores, or any other suitable material that can be used in imaging. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, ZQ800, or indocyanine green (ICG). In some aspects, near infrared dyes often include cyanine dyes. Additional non-limiting examples of fluorescent dyes for use as a conjugating molecule in the present disclosure include acradine orange or yellow, Alexa Fluors and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DApI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

The peptides of the present disclosure can also be conjugated to other moieties that can serve other roles, such as providing an affinity handle (e.g., biotin) for retrieval of the peptides from tissues or fluids. For example, the peptides of the present disclosure can also be conjugated to biotin. Biotin can also act as an affinity handle for retrieval of peptides from tissues or other locations. In some embodiments, fluorescent biotin conjugates that can act both as a detectable label and an affinity handle can be used. Non-limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa Fluor 488 biocytin, Alexa Fluor 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates can include chemiluminescent compounds, colloidal metals, luminescent compounds, enzymes, radioisotopes, and paramagnetic labels.

### Linkers

As discussed above and herein, the peptides of the present disclosure can be conjugated to another moiety (e.g., an active agent), such as a small molecule, a second peptide, a protein, an antibody, an antibody fragment, a single chain Fv, an aptamer, polypeptide, polynucleotide, a fluorophore, a radioisotope, a radionuclide chelator, a polymer, a biopolymer, a fatty acid, an acyl adduct, a chemical linker, or sugar or other active agent described herein through a linker, or directly, in the absence of a linker. Direct attachment is possible by covalent attachment of a peptide to a region of the larger molecule. For example, in some embodiments, the peptide is attached to a terminus of the amino acid sequence of the larger molecule, or could be attached to a side chain, such as the side chain of a lysine, serine, threonine, cysteine, tyrosine, aspartic acid, a non-natural amino acid residue, or glutamic acid residue. The attachment can be via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond, or linker as described herein) can be used to link other molecules.

In certain embodiments, attachment via a linker involves incorporation of a linker moiety between the larger molecule and the peptide. The peptide and the larger molecule can both be covalently attached to the linker. The linker can be cleavable, non-cleavable, self-immolating, hydrophilic, or hydrophobic. In various embodiments, the linker has at least two functional groups, one bonded to the larger molecule, and one bonded to the peptide, and a linking portion between the two functional groups.

Non-limiting examples of the functional groups for attachment include functional groups capable of forming, for example, an amide bond, an ester bond, an ether bond, a carbonate bond, a carbamate bond, or a thioether bond. Non-limiting examples of functional groups capable of forming such bonds include amino groups; carboxyl groups; aldehyde groups; azide groups; alkyne and alkene groups; ketones; hydrazides; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion include alkylene, alkenylene, alkynylene, polyether, such as polyethylene glycol (PEG), oligoethylene glycol, polyester, polyamide, polyamino acids, polypeptides, cleavable peptides, valine-citrulline, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, and ester groups.

Non-limiting examples of linkers include:

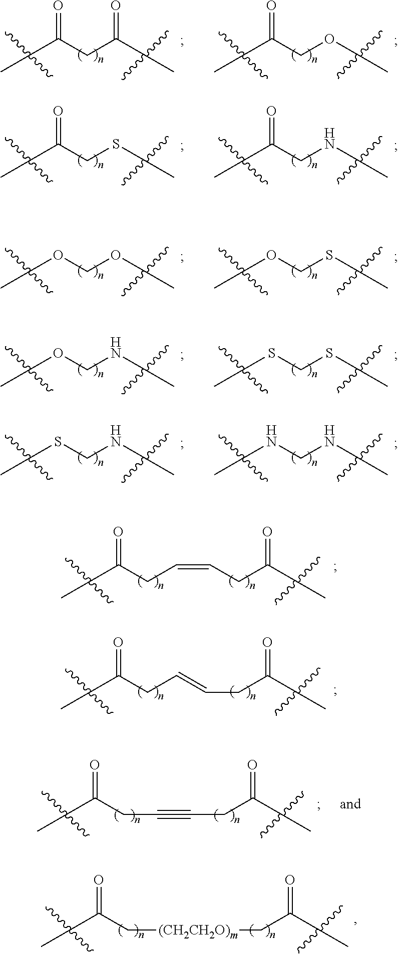

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50.

In some embodiments, the linker is a succinic linker, and a moiety is attached to a peptide via an ester bond or an amide bond with two methylene carbons in between. In other cases, a linker can be any linker with both a hydroxyl group and a carboxylic acid, such as hydroxy hexanoic acid or lactic acid.

The linker can be a cleavable linker or a noncleavable linker. A noncleavable linker can be referred to as a “stable” linker. In some embodiments, the linker is enzyme cleavable, e.g., a valine-citrulline linker. In some embodiments, the linker contains a self-immolating portion. In some embodiments, the linker includes one or more cleavage sites for a specific protease, such as a cleavage site for matrix metalloproteases (MMPs), thrombin, or cathepsin. Alternatively or in combination, the linker is cleavable by other mechanisms, such as via pH, reduction, thiol exchange, or hydrolysis. The use of a cleavable linker permits release of the conjugated moiety (e.g., a therapeutic agent) from the peptide, e.g., after targeting to the renal tissue. A hydrolytically labile linker, (amongst other cleavable linkers described herein) can be advantageous in terms of releasing active agents from the peptide. For example, an active agent in a conjugate form with the peptide may not be active, but upon release from the conjugate after targeting to the renal tissue, the active agent is active. Alternatively, a stable linker can still permit release of an active cleavage product after catabolism in a cell.

In some embodiments, a peptide can be conjugated to an active agent by common techniques known in the art, such those described in Bioconjugate Techniques by Greg T. Hermanson (2013).

The rate of hydrolysis of the linker can be tuned. For example, the rate of hydrolysis of linkers with unhindered esters is faster compared to the hydrolysis of linkers with bulky groups next an ester carbonyl. As additional examples, the rate of disulfide cleavage or exchange with unhindered disulfides is faster compared to the rate of disulfide cleavage or exchange of linkers with bulky groups near disulfide bonds. Protease sites can also affect cleavage rates. A bulky group can be a methyl group, an ethyl group, a phenyl group, a ring, or an isopropyl group, or any group that provides steric bulk. In some cases, the steric bulk can be provided by the drug itself, such as by ketorolac when conjugated via its carboxylic acid. The rate of hydrolysis of the linker can be tuned according to the residency time of the conjugate in the kidneys. For example, when a peptide is cleared from the kidneys relatively quickly, the linker can be tuned to rapidly hydrolyze. In contrast, for example, when a peptide has a longer residence time in the kidneys, a slower hydrolysis rate can allow for extended delivery of an active agent. This can be important when the peptide is used to deliver a drug to the kidneys. “Programmed hydrolysis in designing paclitaxel prodrug for nanocarrier assembly” Sci Rep 2015, 5, 12023 Fu et al., provides an example of modified hydrolysis rates.

### Peptide Stability

A peptide of the present disclosure can be stable in various biological conditions. For example, any peptide of SEQ ID NO: 1-SEQ ID NO: 118 can exhibit resistance to reducing agents, proteases, oxidative conditions, or acidic conditions.

In some cases, biologic molecules (such as peptides and proteins) can provide therapeutic functions, but such therapeutic functions are decreased or impeded by instability caused by the in vivo environment. (Moroz et al. Adv Drug Deliv Rev 101:108-21 (2016), Mitragotri et al. Nat Rev Drug Discov 13(9):655-72 (2014), Bruno et al. Ther Deliv (11):1443-67 (2013), Sinha et al. Crit Rev Ther Drug Carrier Syst. 24(1):63-92 (2007), Hamman et al. BioDrugs 19(3):165-77 (2005)). For instance, the GI tract can contain a region of low pH (e.g. pH ˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering reasonable dosing regimens. Likewise, proteolytic activity in cellular compartments such as lysosomes and reduction activity in lysosomes and the cytosol can degrade peptides and proteins such that they may be unable to provide a therapeutic function on intracellular targets. Therefore, peptides that are resistant to reducing agents, proteases, and low pH may be able to provide enhanced therapeutic effects or enhance the therapeutic efficacy of co-formulated or conjugated active agents in vivo.

Additionally, oral delivery of drugs can be desirable in order to target certain areas of the body (e.g., disease in the GI tract such as colon cancer, irritable bowel disorder, infections, metabolic disorders, and constipation) despite the obstacles to the delivery of functionally active peptides and polypeptides presented by this method of administration. For example, oral delivery of drugs can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides without nullifying their therapeutic function.

### Peptide Resistance to Reducing Agents

In some embodiments, a knotted peptide of the present disclosure can be reduction resistant. Peptides of this disclosure can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, glutathione (GSH) is a reducing agent that can be present in many areas of the body and in cells, and can reduce disulfide bonds. As another example, a peptide can become reduced upon cellular internalization during trafficking of a peptide across the gastrointestinal epithelium after oral administration A peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life. Reduction of the disulfide bonds can also lead to increased susceptibility of the peptide to subsequent degradation by proteases, resulting in rapid loss of intact peptide after administration. In some embodiments, a peptide that is resistant to reduction can remain intact and can impart a functional activity for a longer period of time in various compartments of the body and in cells, as compared to a peptide that is more readily reduced.

In certain embodiments, the peptides of this disclosure can be analyzed for the characteristic of resistance to reducing agents to identify stable peptides. In some embodiments, the peptides of this disclosure can remain intact after being exposed to different molarities of reducing agents such as 0.00001M-0.0001M, 0.0001M-0.001M, 0.001M-0.01M, 0.01 M-0.05 M, 0.05 M-0.1 M, for greater 15 minutes or more. In some embodiments, the reducing agent used to determine peptide stability can be dithiothreitol (DTT), Tris(2-carboxyethyl)phosphine HCl (TCEP), 2-Mercaptoethanol, (reduced) glutathione (GSH), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a reducing agent.

### Peptide Resistance to Proteases

The stability of peptides of this disclosure can be determined by resistance to degradation by proteases. In some embodiments, a knotted peptide of the present disclosure can be resistant to protease degradation. Proteases, also referred to as peptidases or proteinases, can be enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, esterases, serum proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, the knotted peptides of this disclosure can resist degradation by any class of protease. In certain embodiments, the knotted peptides of this disclosure resist degradation by pepsin (which can be found in the stomach), trypsin (which can be found in the duodenum), serum proteases, or any combination thereof. In certain embodiments, peptides of this disclosure can resist degradation by lung proteases (e.g., serine, cysteinyl, and aspartyl proteases, metalloproteases, neutrophil elastase, alpha-1 antitrypsin, secretory leucoprotease inhibitor, elafin), or any combination thereof. In some embodiments, the proteases used to determine peptide stability can be pepsin, trypsin, chymotrypsin, or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a protease.

### Peptide Stability in Acidic Conditions

Peptides of this disclosure can be administered in biological environments that are acidic. For example, after oral administration, peptides can experience acidic environmental conditions in the gastric fluids of the stomach and gastrointestinal (GI) tract. The pH of the stomach can range from −1-4 and the pH of the GI tract ranges from acidic to normal physiological pH descending from the upper GI tract to the colon. In addition, the vagina, late endosomes, and lysosomes can also have acidic pH values, such as less than pH 7. The pH of various compartments of the kidney can also vary. These acidic conditions can lead to denaturation of peptides and proteins into unfolded states. Unfolding of peptides and proteins can lead to increased susceptibility to subsequent digestion by other enzymes as well as loss of biological activity of the peptide.

In certain embodiments, the peptides of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5-10%, at least 10%-20%, at least 20%-30%, at least 30%-40%, at least 40%-50%, at least 50%-60%, at least 60%-70%, at least 70%-80%, at least 80%-90%, or at least 90-100% of the peptide remains intact after exposure to simulated gastric fluid. In some embodiments, low pH solutions such as simulated gastric fluid or citrate buffers can be used to determine peptide stability.

### Peptide Stability at High Temperatures

In some embodiments, the knotted peptides of the present disclosure are resistant to an elevated temperature. Peptides of this disclosure can be administered in biological environments with high temperatures. For example, after oral administration, peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 25° C. for 6 months to 5 years. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 70° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 100° C. for 15 minutes to 1 hour.

### Methods of Manufacture

Various expression vector/host systems can be utilized for the production of the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g., adenovirus, vaccinia virus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secretes minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, Peptides can also be synthesized in a cell-free system using a variety of known techniques employed in protein and peptide synthesis.

In some cases, a host cell produces a peptide that has an attachment point for a drug. An attachment point could comprise a lysine residue, an N-terminus, a cysteine residue, a cysteine disulfide bond, or a non-natural amino acid. The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can be then be joined together enzymatically or synthetically.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000) or by conventional solution phase peptide synthesis. Refolding and disulfide bond formation can be executed by methods known in the art, such as incubation of the peptide at a mildly basic pH in the presence of a redox pair such as reduced and oxidized cysteine, either after cleavage and protecting group removal and purification, or while still on the resin. Peptide fragments can also be made synthetically or recombinantly and then joined together.

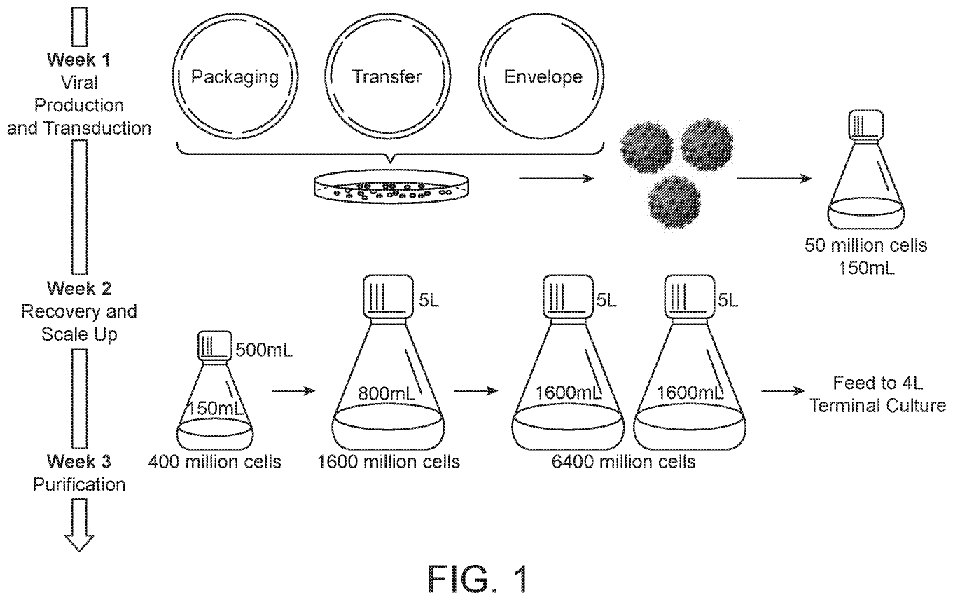

FIG. 1 illustrates a brief schematic of a method of manufacturing a construct that expresses a peptide of the disclosure.

### Pharmaceutical Compositions of Peptides and Peptide-Conjugates

A pharmaceutical composition of the disclosure can be a combination of any peptide or peptide-conjugate described herein, or a salt thereof, with other chemical components, such as carriers, stabilizers, diluents, dispersing agents, suspending agents, thickening agents, and/or excipients. In some embodiments, the pharmaceutical composition facilitates administration of a peptide or peptide-conjugate described herein to an organism. Pharmaceutical compositions can be formulated for administration to a subject by various routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, or topical administration, or a combination thereof. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduces the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously.

A peptide or peptide-conjugate of the disclosure can be applied directly to an organ, or an organ tissue or cells, such as renal tissue or cells, during a surgical procedure. The peptides or peptide-conjugates described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptides or peptide-conjugates described herein described herein can be administered in pharmaceutical compositions to a subject suffering from a condition that affects the renal system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of the pharmaceutical compositions described herein include formulating the peptide or peptide-conjugate described herein, or a salt thereof, with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

### Administration of Pharmaceutical Compositions

A pharmaceutical composition of the disclosure can be a combination of any plant, venom, toxin or artifically derived disulfide-rich peptide described herein with other chemical components, such as carriers, stabilizers, diluents, dispersing agents, suspending agents, thickening agents, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, inhalation, dermal, intrathecal, intranasal, and topical administration. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduces the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously. A peptide described herein can be administered to a subject, homes, targets, is directed to, accumulates in, migrates to, is retained by, or binds to an organ, e.g., the kidneys.

A peptide of the disclosure can be applied directly to an organ, or an organ tissue or cells, such as the kidneys or renal tissue or cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein are administered in pharmaceutical compositions to a subject suffering from a condition. In some instances the pharmaceutical composition will affect the physiology of the animal, such as the immune system, inflammatory response, or other physiologic affect. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Use of Peptide in Imaging and Surgical Methods

In some embodiments, the present disclosure provides a method for detecting a cancer, cancerous tissue, or tumor tissue, the method comprising the steps of contacting a tissue of interest with a peptide of the present disclosure, wherein the peptide is conjugated to a detectable agent and measuring the level of binding of the peptide, wherein an elevated level of binding, relative to normal tissue, is indicative that the tissue is a cancer, cancerous tissue or tumor tissue.

In some embodiments, the disclosure provides a method of imaging an organ or body region or region, tissue or structure of a subject, the method comprising administrating to the subject the peptide or a pharmaceutical composition disclosed herein and imaging the subject. In some embodiments such imaging is used to detect a condition associated with a function of the kidneys. In some cases the condition is an inflammation, a cancer, a degradation, a growth disturbance, genetic, a tear or an injury, or another suitable condition. In some case the condition is associated with a cancer or tumor of the kidneys. In some embodiments, such as those associated with cancers, the imaging can be associated with surgical removal of the diseased region, tissue, structure or cell of a subject.

Furthermore, the present disclosure provides methods for intraoperative imaging and resection of a diseased or inflamed tissue, cancer, cancerous tissue, or tumor tissue using a peptide of the present disclosure conjugated with a detectable agent. In some embodiments, the diseased or inflamed tissue, cancer, cancerous tissue, or tumor tissue is detectable by fluorescence imaging that allows for intraoperative visualization of the cancer, cancerous tissue, or tumor tissue using a peptide of the present disclosure. In some embodiments, the peptide of the present disclosure is conjugated to one or more detectable agents. For example, 1, 2, 3, 4, 5, 6, 7, 8, 9, or 10 detectable agents can be conjugated to a peptide of this disclosure. In a further embodiment, the detectable agent comprises a fluorescent moiety coupled to the peptide. In another embodiment, the detectable agent comprises a radionuclide. In some embodiments, imaging is achieved during open surgery. In further embodiments, imaging is accomplished using endoscopy or other non-invasive surgical techniques.

Renal Therapy with Peptides and Peptide-Conjugates

As discussed above and herein, the present disclosure provides peptides that home, target, migrate to, accumulate in, are directed to, and/or bind to specific regions, tissues, structures, or cells of the kidney and methods of using such peptides. End uses of such peptides include, for example, imaging, research, therapeutics, diagnostics, theranostics, pharmaceuticals, chemotherapy, chelation therapy, targeted drug delivery, and radiotherapy.

In one embodiment, the method includes administering an effective amount of a peptide of the present disclosure to a subject in need thereof. The term “effective amount,” as used herein, can refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case can be determined using techniques, such as a dose escalation study.

Multiple peptides or peptide-conjugates described herein can be administered in any order or simultaneously. For example, in some embodiments, multiple functional fragments of peptides derived from toxins or venom can be administered in any order or simultaneously. If simultaneously, the multiple peptides or peptide-conjugates described herein can be provided in a single, unified form, such as an intravenous injection, or in multiple forms, such as subsequent intravenous dosages.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal, or a lab animal afflicted with a disease or condition) with a peptide of the disclosure. The disease can be a renal disease. The disease can be treated as a result of the subject's renal tissue uptake of the peptide. The subject can be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment can be a prophylactic treatment provided to the subject. Treatment can be provided to the subject after clinical onset of disease. Treatment can be provided to the subject after 1 day, 1 week, 6 months, 12 months, or 2 years or more after clinical onset of the disease. Treatment can be provided to the subject for more than 1 day, 1 week, 1 month, 6 months, 12 months, 2 years or more after clinical onset of disease. Treatment can be provided to the subject for less than 1 day, 1 week, 1 month, 6 months, 12 months, or 2 years or more after clinical onset of the disease. Treatment can be administered daily, weekly, monthly or yearly. Treatment can also include treating a human in a clinical trial. A treatment can comprise administering to a subject a pharmaceutical composition, such as one or more of the pharmaceutical compositions described throughout the disclosure. A treatment can comprise delivering a peptide of the disclosure to a subject, either intravenously, subcutaneously, intramuscularly, by inhalation, dermally, topically, orally, sublingually, intrathecally, transdermally, intranasally, or via a peritoneal route. A treatment can comprise administering a peptide-active agent complex to a subject, either intravenously, subcutaneously, intramuscularly, by inhalation, dermally, topically, orally, intrathecally, transdermally, intransally, parenterally, orally, via a peritoneal route, nasally, or sublingually.

The various peptides and peptide-conjugates described herein can be used as therapy and administered for therapeutic applications, e.g., to a subject already suffering from a disease or condition, in an amount sufficient to cure or at least partially arrest the symptoms of the disease or condition, or to cure, heal, improve, or ameliorate the condition. As used herein, the terms “therapy” and “therapeutic” also encompass protective, preventative, and/or prophylactic applications, e.g., administration of a peptide or peptide-conjugate to a subject in order to prevent (either in whole or in part) or lessen a likelihood of developing, contracting, or worsening a condition. Amounts effective for this use can vary based on the severity and course of the disease or condition, previous therapy, the subject's health status, weight, and response to the drugs, and the judgment of the treating physician. As used herein, in some embodiments, the terms “therapy” and “therapeutic” can also be used in conjunction with aspects of a therapy or therapeutic effect to aid in understanding, for example, “renal therapeutic,” “chemotherapy,” or “chemotherapeutic,” as non-limiting examples.

In some embodiments, the peptides and peptide-conjugates of the present disclosure are used to treat a condition of the kidney, or a region, tissue, structure, or cell thereof. In certain embodiments, the condition is associated with a function of a subject's kidneys. The present disclosure encompasses various acute and chronic renal diseases, including glomerular, tubule-interstitial, and microvascular diseases. Examples of conditions applicable to the present disclosure include but are not limited to: acute kidney diseases and disorders (AKD), acute kidney injury (AKI) due to cardiovascular surgery, radiocontrast nephropathy, or induced by cisplatin or carboplatin, which can be treated prophylactically, established AKI including ischemic renal injury, endotoxemia-induced AKI, endotoxemia/sepsis syndrome, or established nephrotoxic AKI (e.g., rhabdomyolysis, radiocontrast nephropathy, cisplatin/carboplatin AKI, aminoglycoside nephrotoxicity), end stage renal disease, acute and rapidly progressive glomerulonephritis, acute presentations of nephrotic syndrome, acute pyelonephritis, acute renal failure, chronic glomerulonephritis, chronic heart failure, chronic interstitial nephritis, graft versus host disease after renal tranplant, chronic kidney disease (CKD) such as diabetic nephropathy, hypertensive nephrosclerosis, idiopathic chronic glomerulonephritis (e.g., focal glomerular sclerosis, membranous nephropathy, membranoproliferative glomerulonephritis, minimal change disease transition to chronic disease, anti-GBM disease, rapidly progressive cresentic glomerulonephritis, IgA nephropathy), secondary chronic glomerulonephritis (e.g., systemic lupus, polyarteritis nodosa, scleroderma, amyloidosis, endocarditis), hereditary nephropathy (e.g., polycystic kidney disease, Alport's syndrome), interstitial nephritis induced by drugs (e.g., Chinese herbs, NSAIDs), multiple myeloma or sarcoid, or renal transplantation such as donor kidney prophylaxis (treatment of donor kidney prior to transplantation), treatment post transplantation to treat delayed graft function, acute rejection, or chronic rejection, chronic liver disease, chronic pyelonephritis, diabetes, diabetic kidney disease, fibrosis, focal segmental glomerulosclerosis, Goodpasture's disease, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, renal fibrosis, and vasculitis. For example, in certain embodiments, the peptides and peptide-conjugates of the present disclosure are used to reduce acute kidney injury in order to prevent it from progressing to chronic kidney disease.

Alternatively or in combination, in some embodiments, the peptide and peptide-conjugates of the present disclosure are used to elicit a protective response such as ischemic preconditioning and/or acquired cytoresistance in a kidney of the subject. In some embodiments, ischemic preconditioning and/or acquired cytoresistance is induced by administering an agent (e.g., a peptide or peptide-conjugate of the present disclosure) that upregulates the expression of protective stress proteins, such as antioxidants, anti-inflammatory proteins, or protease inhibitors. In certain embodiments, the induced response protects the kidney by preserving kidney function in whole or in part and/or by reducing injury to renal tissues and cells, e.g., relative to the situation where no protective response is induced. The peptides and peptide-conjugates of the present disclosure can provide certain benefits compared to other agents for inducing ischemic preconditioning and/or acquired cytoresistance, such as a well-defined chemical structure and avoidance of low pH precipitation.

In some embodiments, the protective response is induced in order to protect the kidney or tissues or cells thereof from an injury or insult that is predicted to occur (e.g., associated with a planned event such as a medical procedure, is likely to occur due to a condition in the subject) or has already occurred. In certain embodiments, the induced response prevents or reduces the extent of damage to the kidney or tissues or cells thereof caused by the injury or insult. For instance, in certain embodiments, the peptides and peptide-conjugates induce acquired cytoresistance by activating protective pathways and/or upregulating expression of protective stress proteins. Optionally, the peptides and peptide-conjugates are capable of inducing such protective responses while causing minimal or no injury to the kidney.

In various embodiments, the injury or insult is associated with one or more of: surgery, radiocontrast imaging, cardiopulmonary bypass, balloon angioplasty, induced cardiac or cerebral ischemic-reperfusion injury, organ transplantation, sepsis, shock, low blood pressure, high blood pressure, kidney hypoperfusion, chemotherapy, drug administration, nephrotoxic drug administration, blunt force trauma, puncture, poison, or smoking. For instance, in certain embodiments, the injury or insult is associated with a medical procedure that has been or will be performed on the subject, such as one or more of: surgery, radiocontrast imaging, cardiopulmonary bypass, balloon angioplasty, induced cardiac or cerebral ischemic-reperfusion injury, organ transplantation, chemotherapy, drug administration, or nephrotoxic drug administration.

In some embodiments, the peptide itself exhibits a renal therapeutic effect. For example, in certain embodiments, the knotted peptide interacts with a renal ion channel, inhibits a protease, has antimicrobial activity, has anticancer activity, has anti-inflammatory activity, induces ischemic preconditioning or acquired cytoresistance, or produces a protective or therapeutic effect on a kidney of the subject, or a combination thereof. Optionally, the renal therapeutic effect exhibited by the peptide is a renal protective effect or renal prophylactic effect (e.g., ischemic preconditioning or acquired cytoresistance) that protects the kidney or a tissue or cell thereof from an upcoming injury or insult.

For example, in certain embodiments, a peptide of the present disclosure activates protective pathways and/or upregulates expression of protective stress proteins in the kidney or tissues or cells thereof. As another example, in certain embodiments, a peptide of the present disclosure accesses and suppresses intracellular injury pathways. In yet another example, in certain embodiments, a peptide of the present disclosure inhibits interstitial inflammation and prevents renal fibrosis. As a further example, in certain embodiments, a peptide of the present disclosure is administered prior to or currently with the administration of a nephrotoxic agent (e.g., aminoglycoside antibiotics such as gentamicin and minocycline, chemotherapeutics such as cisplatin, immunoglobulins or fragments thereof, mannitol, NSAIDs such as ketorolac or ibuprofen, cyclosporin, cyclophosphamide, radiocontrast dyes) in order to minimize its damaging effects, e.g., by blocking megalin-cubulin binding sites so that the nephrotoxic agent passes through the kidneys.

Alternatively or in combination, in some embodiments, the peptide is conjugated to a renal therapeutic agent that exhibits a renal therapeutic effect. In certain embodiments, the renal therapeutic agent is used to treat a condition of the kidney, or a region, tissue, structure, or cell thereof, such as the conditions provided herein. Examples of such renal therapeutic agents include but are not limited to: dexamethasone, a steroid, an anti-inflammatory agent, an antioxidant (e.g., glutathione, N acetyl cysteine), deferoxamine, feroxamine, iron, tin, a metal, a metal chelate, ethylene diamine tetraacetic acid (EDTA), an EDTA-Fe complex, dimercaptosuccinic acid (DMSA), 2,3-dimercapto-1-propanesulfonic acid (DMPS), penicillamine, an antibiotic such as gentamicin, vancomycin, minocin or mitomyclin, an iron chelator, a porphyrin, hemin, vitamin B12, a chemotherapeutic, an Nrf2 pathway activator such as bardoxolone, angiotensin-converting-enzyme (ACE) inhibitors such as ramipril, captopril, lisinopril, benazepril, quinapril, fosinopril, trandolapril, moexipril, enalaprilat, enalapril maleate, or perindopril erbumine, glycine polymers, or a combination thereof. Additional examples of a therapeutic agent that can be conjugated to the peptide can include QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO and EPO derivatives, agents that stimulate erthyropoietin such as epoeitn alfa or darbepoietin alfa, PDGF inhibitors, CRMD-001, Atrasentan, Tolvaptan, RWJ-676070, Abatacept, Sotatercept, an anti-infective agent, an anti-viral agent, an anti-fungal agent, an aminoglycoside, an immunosuppresant such tacrolimus, mycophenolic acid (e.g., mycophenolate mofetil), cyclosporine A, or azathioprine, a diuretic drug such as thiazides, bemetanide, ethacrynic acid, furosemidem torsemide, glucose, mannitol, amiloride, spironolactone, eplerenone, triamterene, potassium canrenoate, bendroflumethiazide, hydrochlorothiazide, vasopressin, amphotericin B, acetazolamide, tovaptan, conivaptan, dopamine, dorzolamide, bendrolumethiazide, hydrochlorothiazide, caffeine, theophylline, or theobromine, a statin, a senolytic such as navitoclax or obatoclax, a corticosteroid such as prednisone, betamethasone, fludrocortisone, deoxycorticosterone, aldosterone, cortisone, hydrocortisone, belcometasone, dexamethasone, mometasone, fluticasone, prednisolone, methylprednisolone, triamcinolone acetonide or triamcinolone, a glucocorticoid, a liposome, renin, SGLT2 modulator, or angiotensin.

For example, in some embodiments, a peptide of the present disclosure is conjugated to an anti-inflammatory agent such as dexamethasone in order to treat lupus affecting the kidney, vasculitis, Goodpasture's disease, focal segmental glomerulosclerosis, nephritic syndrome, or other renal disorders caused by inflammatory processes. As another example, in some embodiments, a peptide of the present disclosure is conjugated to chemotherapeutic for treating renal cell carcinoma. As a further example, in some embodiments, a peptide of the present disclosure is conjugated to a steroid for treating polycystic renal disease.

In certain embodiments, the renal therapeutic agent is a renal protective agent or renal prophylactic agent capable of eliciting a protective response in the kidney upon administration to a subject. As discussed above and herein, the protective response can protect the kidney or a tissue or cell thereof from an upcoming injury or insult. For example, the renal protective agent or renal prophylactic agent can activate protective pathways and/or upregulate expression of protective stress proteins in the kidney or tissues or cells thereof. Examples of such renal protective agents and renal prophylactic agents include but are not limited to: dexamethasone, a steroid, an anti-inflammatory agent, a nonsteroidal anti-inflammatory drug (NSAID) such as ketorolac or ibuprofen, deferoxamine, iron, tin, a metal, a metal chelate, ethylene diamine tetraacetic acid (EDTA), an EDTA-Fe complex, dimercaptosuccinic acid (DMSA), 2,3-dimercapto-1-propanesulfonic acid (DMPS), penicillamine, an antibiotic, an aminoglycoside, an iron chelator, a porphyrin, vitamin B12, or a combination thereof. In some embodiments, the renal protective agent or renal prophylactic agent comprises complexed or chelated iron, (e.g., via heme, deferoxamine, feroxamine, porphyrin, EDTA, etc.). In such embodiments, the peptide-conjugate can be used to deliver iron to the renal tissue for kidney preconditioning.

For example, in certain embodiments, a peptide of the present disclosure is conjugated to hemin, which signals through the heat shock/heme reactive element pathway in order to upregulate a set of diverse cytoprotective proteins. As another example, in certain embodiments, a peptide of the present disclosure is conjugated to an iron chelate or iron complex in order to deliver iron to the kidney to alter gene expression profiles and induce expression of cytoprotective proteins.

The peptides of the present disclosure enable specific targeting of renal therapeutic agents and other agents to the kidneys, which in some embodiments is beneficial for reducing undesirable effect associated with systemic delivery and/or delivery to non-target tissues. For example, patients with inflammation-driven renal diseases that are currently treated with systemic steroids can benefit from peptide-steroid conjugates of the present disclosure that would deliver the therapeutic specifically to the kidneys at sufficiently high concentrations to elicit a targeted therapeutic effect, while reducing acute systemic side effects. In patients suffering from chronic disease, this approach can advantageously spare much of the rest of the body from side effects associated with long-term use of steroidal compounds. As another example, the peptide-conjugates of the present disclosure can be used for targeted delivery of iron for kidney preconditioning, thus reducing or preventing toxicity associated with systemic iron delivery.

In some embodiments, a method of treating a condition in a subject in need thereof comprises administering to the subject a composition or pharmaceutical composition comprising any of the peptides or peptide-conjugates described herein. For example, in certain embodiments, the composition comprises any of the peptides described herein, such as a knotted peptide. Optionally, the composition comprises a moiety coupled to the peptide, such as an active agent (e.g., a renal therapeutic agent) or any other moiety described herein. In various embodiments, the pharmaceutical composition comprises any composition of the present disclosure or a salt thereof, and any of pharmaceutically acceptable carriers described herein. In various embodiments, the composition or pharmaceutical composition homes, targets, is directed to, accumulates in, migrates to, is retained by, or binds to the renal tissue of the subject following administration. The composition or pharmaceutical composition can provide a therapeutic effect on the renal tissue in order to treat the condition, as discussed above and herein.

In some embodiments, a method of protecting a kidney of a subject from injury comprises administering to the subject a composition or pharmaceutical composition comprising any of the peptides or peptide-conjugates described herein. For example, in certain embodiments, the composition comprises any of the peptides described herein, such as a knotted peptide. Optionally, the composition comprises a moiety coupled to the peptide, such as an active agent (e.g., a renal therapeutic agent) or any other moiety described herein. In various embodiments, the pharmaceutical composition comprises any composition of the present disclosure or a salt thereof, and any of pharmaceutically acceptable carriers described herein.

In some embodiments, the method further comprises inducing ischemic preconditioning and/or acquired cytoresistance in the kidney of the subject. The ischemic preconditioning and/or acquired cytoresistance can protect the kidney from an injury or insult, as described above and herein. The methods of the present disclosure allow such protective responses to be preemptively induced in order to protect the kidney from an upcoming injury or insult. For example, in certain embodiments, the composition or pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours prior to a predicted occurrence of the injury or insult.

Alternatively or in combination, the present disclosure includes methods for inducing a protective response in order to treat an injury or insult that has already occurred. For example, in certain embodiments, the composition or pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours after an occurrence of the injury or insult.

In some embodiments, the method further comprises performing a medical procedure on the subject. The medical procedure can potentially cause injury or insult to the subject's kidneys. The method of the present disclosure can be used to induce a protective response in order to protect the kidneys from an injury or insult associated with an upcoming medical procedure. For example, in certain embodiments, the composition or the pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours prior to performing the medical procedure.

Alternatively or in combination, the present disclosure includes methods for inducing a protective response in order to treat an injury or insult associated with a medical procedure that has already been performed on the subject. For example, in certain embodiments, the composition or the pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours after performing the medical procedure.

Peptides, peptide-conjugates, and/or pharmaceutical compositions can be packaged as a kit. In some embodiments, a kit includes written instructions on the use or administration of the peptides, peptide-conjugates, and/or pharmaceutical compositions, in accordance with the various methods described herein.

As used herein the term “and/or” is used as a functional word to indicate that two words or expressions are to be taken together or individually. For example, A and/or B encompasses A alone, B alone, and A and B together.

All features discussed in connection with any embodiment or embodiment herein can be readily adapted for use in other embodiments and embodiments herein. The use of different terms or reference numerals for similar features in different embodiments does not necessarily imply differences other than those expressly set forth. Accordingly, the present disclosure is intended to be described solely by reference to the appended claims, and not limited to the embodiments disclosed herein.

Unless otherwise specified, the presently described methods and processes can be performed in any order. For example, a method describing steps (a), (b), and (c) can be performed with step (a) first, followed by step (b), and then step (c). Or, the method can be performed in a different order such as, for example, with step (b) first followed by step (c) and then step (a), or any combinations thereof. Furthermore, such steps can be performed in combination with additional steps or methods. Furthermore, those steps can be performed simultaneously or separately unless otherwise specified with particularity.

The particulars shown herein are by way of example and for purposes of illustrative discussion of the preferred embodiments of the present disclosure only and are presented in the cause of providing what is believed to be the most useful and readily understood description of the principles and conceptual embodiments of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

While preferred embodiments of the present disclosure have been shown and described herein, it is to be understood that the disclosure is not limited to the particular embodiments of the disclosure described, as variations of the particular embodiments can be made and still fall within the scope of the appended claims. It is also to be understood that the terminology employed is for the purpose of describing particular embodiments of the disclosure, and is not intended to be limiting. Instead, the scope of the present disclosure is established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the disclosure provided herein.

All features discussed in connection with an embodiment or embodiment herein can be readily adapted for use in other embodiments and embodiments herein. The use of different terms or reference numerals for similar features in different embodiments does not necessarily imply differences other than those expressly set forth. Accordingly, the present disclosure is intended to be described solely by reference to the appended claims, and not limited to the embodiments disclosed herein.

### EXAMPLES

The following examples are included to further describe some aspects of the present disclosure, and should not be used to limit the scope of the invention

### Example 1

### Manufacture of Peptides in a Mammalian System

This example describes the manufacture of peptides of this disclosure in a mammalian system as briefly shown by FIG. 1. Knotted peptides were generated in mammalian cell culture using a published methodology. (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

The peptide sequence was reverse-translated into DNA, synthesized, and cloned in-frame with siderocalin using standard molecular biology techniques. (M. R. Green, Joseph Sambrook, Molecular Cloning, 2012 Cold Spring Harbor Press). The resulting construct was packaged into a lentivirus, transfected into HEK293 cells, expanded, isolated by immobilized metal affinity chromatography (IMAC), cleaved with tobacco etch virus protease, and purified to homogeneity by reverse-phase chromatography. Following purification, peptides were lyophilized and stored frozen.

### Example 2

### Radiolabeling of Peptide

This example describes radiolabeling of peptides of this disclosure. Several knotted peptides were radiolabeled by reductive methylation with ¹⁴C formaldehyde and sodium cyanoborohydride with standard techniques. The sequences were engineered to have the amino acids, “G” and “S” at the N terminus. See Methods in Enzymology V91:1983 p. 570 and JBC 254(11):1979 p. 4359. An excess of formaldehyde was used to ensure complete methylation (dimethylation of every free amine). The labeled peptides were isolated via solid-phase extraction on Strata-X columns (Phenomenex 8B-S100-AAK), rinsed with water with 5% methanol, and recovered in methanol with 2% formic acid. Solvent was subsequently removed in a blowdown evaporator with gentle heat and a stream of nitrogen gas. The final product was verified and characterized by high performance liquid chromatography (HPLC).

### Example 3

### Accumulation of Peptide in Renal Tissue

This example describes accumulation of peptides of this disclosure in renal tissue. ¹⁴C-methylated knotted peptides were intravenously dosed into mice at 30-100 nmol per mouse. After 4-24 hours in circulation, deeply anesthesized mice were euthanized by freezing in dry ice-chilled hexane. Cryosectioning was performed on a Bright-Hacker cryotome, taking 40 μm sagittal sections. Collected sections were allowed to freeze dry at −20° C. for 48-72 hours before being exposed to phosphor imager plates. Plates were exposed for 7 days then scanned on a RayTest CR-Bio35 scanner. Analysis was performed with AIDA WBA analysis software.

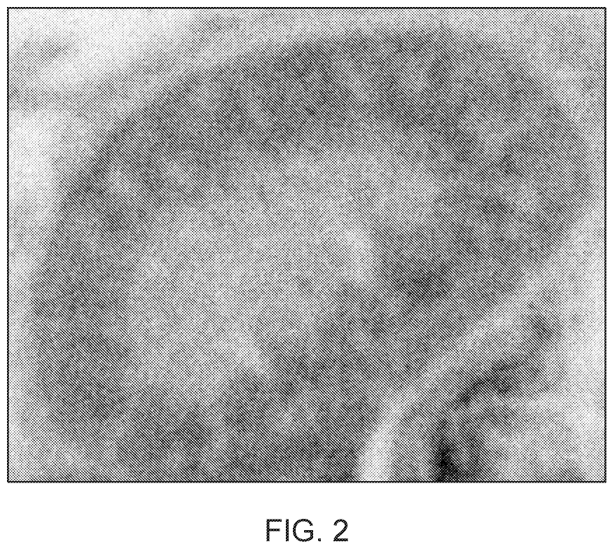

FIG. 2 illustrates the renal signal pattern for the fluoxetine control, which demonstrates non-interactive passage through the kidneys.

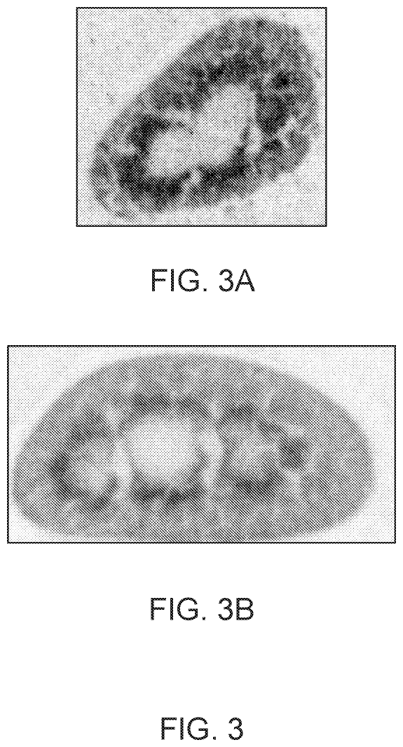

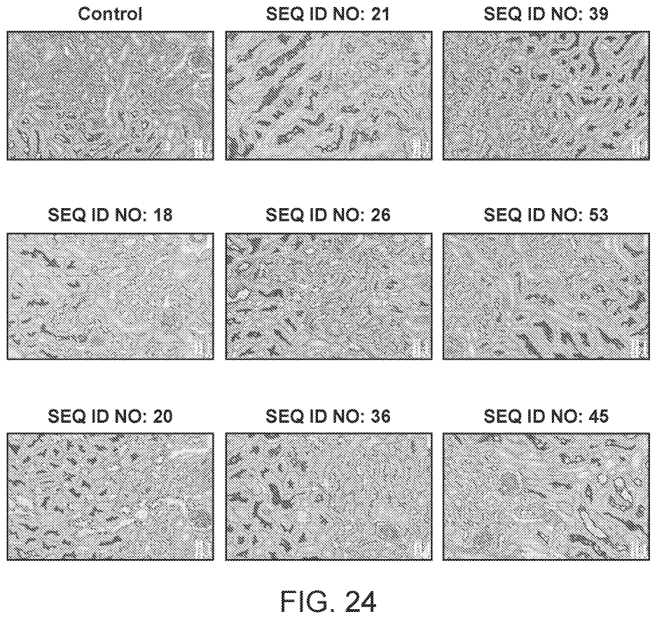

FIGS. 3A and 3B show accumulation of ¹⁴C signal for a peptide of SEQ ID NO: 4 at two time points, 3 hours (FIG. 3A) and 24 hours (FIG. 3B). This data suggests that the peptide is interacting with the kidney, likely cells of the proximal tubule. It is anticipated that freely filtered proteins would not display a persistent signal in the kidneys as observed here.

### Example 4

### Renal Biocompatibility of Peptides

This example describes renal biocompatibility of peptides of this disclosure. The peptides of SEQ ID NO: 18, SEQ ID NO: 20, SEQ ID NO: 21, SEQ ID NO: 26, SEQ ID NO: 36, SEQ ID NO: 39, SEQ ID NO: 45 and SEQ ID NO: 53 were intravenously dosed into mice at 100 nmol per mouse. After 24 hours, mice were anesthesized with Ketamine-Xylazine and euthanized. Plasma and kidneys were removed as quickly as possible after euthanization. A blood urea nitrogen (BUN) assay was performed to assess renal toxicity with a commercially available kit.

The results of the BUN assay for plasma are shown below in TABLE 2. The average BUN concentration in plasma for naïve mice was 25 mg/dL, with a standard error of 2 mg/dL. The average BUN concentration in plasma for peptide-dosed mice was 21, with a standard error of 1 mg/dL. This data demonstrates that none of the eight tested peptides exhibited renal toxicity.

TABLE 2
BUN concentration in plasma

| Naïve | Peptide | |
|---|---|---|
| BUN (mg/dL) | BUN (mg/dL) | SEQ ID NO |
| 25 | 21 | SEQ ID NO: 18 |
| 22 | 18 | SEQ ID NO: 18 |
| 28 | 29 | SEQ ID NO: 20 |
| | 21 | SEQ ID NO: 20 |
| | 23 | SEQ ID NO: 21 |
| | 17 | SEQ ID NO: 21 |
| | 19 | SEQ ID NO: 26 |
| | 22 | SEQ ID NO: 36 |
| | 21 | SEQ ID NO: 36 |
| | 23 | SEQ ID NO: 39 |
| | 18 | SEQ ID NO: 39 |
| | 19 | SEQ ID NO: 53 |
| | 22 | SEQ ID NO: 53 |
| | 21 | SEQ ID NO: 45 |
| | 22 | SEQ ID NO: 45 |

### Example 5

### Engineering of a Peptide for Renal Therapy

This example describes engineering of a peptide of this disclosure for renal therapy. A selected knottin (e.g., selected from a library of over 200,000 identified native knottins) is used as a scaffold for a peptide-based therapeutic of the present invention. The peptide is engineered to have two functional elements: (1) homing to the specific site of intended action in the kidney (e.g., glomerulus, proximal tubule); and (2) therapeutic activity (e.g., block an ion channel, reduce inflammation). The peptide can be engineered to exhibit therapeutic activity even in the absence of a conjugated therapeutic. The engineering of the peptide is accomplished by computational design that replaces native amino acids with those selected by computational software or researchers to increase binding and/or activity at the target. Alternatively, mammalian or Pichia display is used, in which many (e.g., tens or hundreds of thousands) of molecules are displayed on cell surfaces, and those with good binders are selected by flow cytometry. The leading candidates (e.g., identified by deep sequencing of flow-captured cells) are then used as the basis for further design. Iterative rounds of evolution using the above and related techniques are used to discover peptides that have both kidney targeting and therapeutic activity in the absence of a “payload” conjugate. The peptides are used in a renal therapy or renal therapeutic application of the present disclosure.

### Example 6

### Treatment of a Kidney Condition with a Peptide of the Disclosure

This example describes treatment of a kidney condition with peptides of this disclosure. A peptide of the disclosure (e.g., any of the peptides of SEQ ID NO: 1-SEQ ID NO: 118) is expressed recombinantly or chemically synthesized. The peptide is administered to a human or animal, where it binds to renal tissue and exhibits a therapeutic effect, e.g., via antioxidant or anti-inflammatory actions. For example, a peptide of the present disclosure is taken up by the proximal tubules, and gains access to and suppresses intracellular injury pathways. As another example, a peptide of the present disclosure migrates to the renal interstitium and inhibits interstitial inflammation and prevents renal fibrosis.

### Example 7

### Treatment of a Kidney Condition with a Peptide-Conjugate of the Disclosure

This example describes treatment of a kidney condition with a peptide-conjugation of this disclosure. A peptide of the disclosure (e.g., any of the peptides of SEQ ID NO: 1-SEQ ID NO: 118) is expressed recombinantly or chemically synthesized. The peptide is then conjugated to a therapeutic agent, such as deferoxamine, dexamethasone, or another anti-inflammatory agent, a chemotherapeutic, or a steroid. Coupling of the therapeutic agent to the peptide targets the therapeutic agent to the kidney. One or more peptide-conjugates are administered to a human or animal. The therapeutic agent is presented in the kidney at adequate concentration to provide a therapeutic effect, such as an antioxidant, anti-inflammatory, or a chemotherapeutic effect. Optionally, the concentration of the therapeutic agent in other tissues is sufficiently low so to cause few or no undesirable side effects.

For example, a peptide of the present disclosure conjugated to dexamethasone or other potent anti-inflammatory agents is used as therapy for lupus affecting the kidney, vasculitis, Goodpasture's disease, focal segmental glomerulo sclerosis, nephritic syndrome, or other renal disorders caused by inflammatory processes.

As another example, a peptide of the present disclosure is used to deliver a chemotherapeutic for treating renal cell carcinoma.

In a further example, a peptide of the present disclosure is used to deliver steroids for treating polycystic renal disease.

### Example 8

### Eliciting a Protective Response in the Kidney with a Peptide of the Disclosure

This peptide describes eliciting a protective response in the kidney with peptides of this disclosure. A peptide of the disclosure (e.g., any of the peptides of SEQ ID NO: 1-SEQ ID NO: 118) is expressed recombinantly or chemically synthesized. The peptide is administered to a human or animal, where it binds to renal tissue and induces ischemic preconditioning or acquired cytoresistance in the kidney. The peptide is administered to the subject prior to an anticipated injury to the kidney, such as surgery or imaging. The injury that occurs to the kidney is reduced by the peptide. Optionally, the progression of acute kidney injury to chronic kidney disease is reduced by the protective response.

### Example 9

### Protecting the Kidney from Nephrotoxic Agents with a Peptide of the Disclosure

This example describes protecting the kidney from nephrotoxic agents with peptides of this disclosure. A peptide of the disclosure (e.g., any of the peptides of SEQ ID NO: 1-SEQ ID NO: 118) is expressed recombinantly or chemically synthesized. The peptide is administered to a human or animal, where it binds to renal tissue, e.g., at megalin-cubulin binding sites. The peptide is administered to the subject prior to or currently with a nephrotoxic agent (e.g., aminoglycoside antibiotics such as gentamicin, vancomycin, and minocycline, chemotherapeutics such as cisplatin, immunoglobulins, mannitol, NSAIDs, cyclosporin, cyclophosphamide, radiocontrast dyes) in order to minimize its damaging effects, e.g., by blocking megalin-cubulin binding sites so that the nephrotoxic agent passes through the kidneys.

### Example 10

### Eliciting a Protective Response in the Kidney with a Peptide-Conjugate of the Disclosure

This example describes eliciting a protective response in the kidney with a peptide-conjugation of this disclosure. A peptide of the disclosure (e.g., any of the peptides of SEQ ID NO: 1-SEQ ID NO: 118) is expressed recombinantly or chemically synthesized. The peptide is then conjugated to a renal protective agent, such as a deferoxamine, or a chelate or porphyrin complex (e.g., hemin, an EDTA-Fe complex). Coupling of the protective agent to the peptide targets the protective agent to appropriate regions of the kidney with a suitable pharmacokinetic profile. One or more peptide-conjugates are administered to a human or animal. The peptide conjugate is administered to the subject prior to an anticipated injury to the kidney, such as surgery or imaging. The renal tissue injury that occurs in the kidney is reduced by the peptide conjugate. Optionally, the progression of kidney injury to chronic kidney disease is reduced by the protective response.

For example, a peptide of the present disclosure is conjugated to hemin, which signals through the heat shock/heme reactive element pathway. Once intracellular localization is achieved, an upregulation of a set of diverse cytoprotective proteins occurs. The peptide-hemin conjugate is administered to a subject who will undergo high-risk surgeries or radiocontrast administration. The peptide-hemin conjugate is administered one day prior to the procedure in order to allow sufficient time for the upregulation of protective proteins to occur.

As another example, a peptide of the present disclosure is used to deliver iron to the kidney, either as a chelate or porphyrin complex, in order to alter gene expression profiles and induce expression of cytoprotective proteins.

### Example 11

### Peptide Budesonide Conjugates

This example describes the conjugation of a peptide of this disclosure to budesonide. The succinic anhydride form of a budesonide (1.3 eq) and 4-dimethylamino pyridine (DMAP, 1.3 eq) were dissolved in acetone with stirring at ambient temperature. After 24 hours the acetone was removed under reduced pressure. The residue was dissolved in ethyl acetate, and washed three times with 0.1 M hydrochloric acid. The organic layer was then washed further with brine and dried over anhydrous sodium sulfate. The ethyl acetate was removed under reduced pressure to leave a gummy residue which was dissolved in 50% acetonitrile (aq), frozen and lyophilized to provide budesonide hemisuccinate as a white powder.

The hemisuccinate was dissolved in 50% dimethylformamide/dimethylsulfoxide in an oven-dried vial along with ethylcarbodiimide hydrochloride (EDC, 1.5 eq) and sulfo-N-hydroxysuccinimide (sulfo-NHS, 1.5 eq). The reaction was stirred at ambient temperature for 2 hours. The crude reaction mixture was used directly in subsequent conjugation reactions.

Peptide-succinate-budesonide conjugates were formed by reacting 1 equivalent of the crude NHS ester with 1 equivalent of peptide dissolved at 2 mg/mL in 50 mM phosphate buffered saline, pH 7.4. The conjugation reaction was monitored by liquid chromatography-mass spectrometry (LC-MS) and once completed was immediately purified on a preparative high-performance liquid chromatography (HPLC) system using a trifluoroacetic acid solvent system.

### Example 12

### Peptide Triamcinolone Acetonide Conjugates

This example describes the conjugation of a peptide of this disclosure to triamcinolone acetonide. The succinic anhydride form of a triamcinolone acetonide (1.3 eq) and 4-dimethylamino pyridine (DMAP, 1.3 eq) were dissolved in acetone with stirring at ambient temperature. After 24 hours the acetone was removed under reduced pressure. The residue was dissolved in ethyl acetate, and washed three times with 0.1 M hydrochloric acid. The organic layer was then washed further with brine and dried over anhydrous sodium sulfate. The ethyl acetate was removed under reduced pressure to leave a gummy residue which was dissolved in 50% acetonitrile (aq), frozen and lyophilized to provide triamcinolone acetonide hemisuccinate as a white powder.

The hemisuccinate is dissolved in 50% dimethylformamide/dimethylsulfoxide in an oven-dried vial along with ethylcarbodiimide hydrochloride (EDC, 1.5 eq) and sulfo-N-hydroxysuccinimide (sulfo-NHS, 1.5 eq). The reaction is stirred at ambient temperature for 2 hours. The crude reaction mixture is used directly in subsequent conjugation reactions.

Peptide-succinate-triamcinolone acetonide conjugates are formed by reacting 1 equivalent of the crude NHS ester with 1 equivalent of peptide dissolved at 2 mg/mL in 50 mM phosphate buffered saline, pH 7.4. The conjugation reaction is monitored by liquid chromatography-mass spectrometry (LC-MS) and once completed is immediately purified on a preparative high-performance liquid chromatography (HPLC) system using a trifluoroacetic acid solvent system.

### Example 13

### Peptide Dexamethasone Conjugates

This example describes the conjugation of a peptide of this disclosure to dexamethasone. The succinic anhydride form of a dexamethasone (1.3 eq) and 4-dimethylamino pyridine (DMAP, 1.3 eq) were dissolved in acetone with stirring at ambient temperature. After 24 hours the acetone was removed under reduced pressure. The residue was dissolved in ethyl acetate, and washed three times with 0.1 M hydrochloric acid. The organic layer was then washed further with brine and dried over anhydrous sodium sulfate. The ethyl acetate was removed under reduced pressure to leave a gummy residue which was dissolved in 50% acetonitrile (aq), frozen and lyophilized to provide dexamethasone hemisuccinate as a white powder.

The hemisuccinate was dissolved in 50% dimethylformamide/dimethylsulfoxide in an oven-dried vial along with ethylcarbodiimide hydrochloride (EDC, 1.5 eq) and sulfo-N-hydroxysuccinimide (sulfo-NHS, 1.5 eq). The reaction was stirred at ambient temperature for 2 hours. The crude reaction mixture was used directly in subsequent conjugation reactions.

Peptide-succinate-dexamethasone conjugates were formed by reacting 1 equivalent of the crude NHS ester with 1 equivalent of peptide dissolved at 2 mg/mL in 50 mM phosphate buffered saline, pH 7.4. The conjugation reaction was monitored by liquid chromatography-mass spectrometry (LC-MS) and once completed was immediately purified on a preparative high-performance liquid chromatography (HPLC) system using a trifluoroacetic acid solvent system.

### Example 14

### Assessment of Renal Injury

This example illustrates assessment of renal injury by peptides of this disclosure. Mice were injected intravenously at a dose of 100 nmol, which is approximately 16 mg/kg. PBS was administered as a negative control. At 24 hours post-peptide administration, mice were euthanized and plasma blood urea nitrogen (BUN) and plasma creatinine were measured. The blood urea nitrogen (BUN) assay was performed to assess renal toxicity with a commercially available kit. Plasma creatinine concentrations were determined using the colorimetrically corrected Jaffe reaction method. Additionally, the kidneys from these mice were removed, sectioned, and stained using periodic acid Schiff (PAS). FIG. 24 shows mice had normal renal physiology 24 hours after intravenous administration of 100 nmol of a peptide of SEQ ID NO: 18, SEQ ID NO: 20, SEQ ID NO: 21, SEQ ID NO: 26, SEQ ID NO: 36, SEQ ID NO: 39, SEQ ID NO: 45, or SEQ ID NO: 53, or a PBS injected negative control. Histological analysis corroborated the mRNA/protein data. TABLE 3 shows plasma BUN and plasma creatinine concentration. Results demonstrated that peptides of this disclosure do not cause renal injury, as determined by the lack of elevation of either BUN or plasma creatinine concentrations.

TABLE 3
Plasma BUN and Creatinine Concentrations

| | Plasma BUN | Plasma Creatinine |
|---|---|---|
| SEQ ID NO | (mg/dL) | (mg/dL) |
| SEQ ID NO: 18 | 19.5 | 0.43 |
| SEQ ID NO: 20 | 25 | 0.5 |
| SEQ ID NO: 21 | 20 | 0.33 |
| SEQ ID NO: 26 | 19 | 0.33 |
| SEQ ID NO: 36 | 21.5 | 0.4 |
| SEQ ID NO: 39 | 20.5 | 0.32 |
| SEQ ID NO: 53 | 20.5 | 0.35 |
| SEQ ID NO: 45 | 21.5 | 0.39 |
| PBS Control | 20 | 0.32 |

At four hours post-peptide administration, potential renal injury was assessed by measuring three stress-induced mRNAs (MCP-1, NGAL, and TNFa). The results were analyzed by simultaneously determining GAPDH product by RT-PCR and are shown in TABLE 4.

TABLE 4
mRNA, Plasma, and Creatinine Levels

| | mMCP-1/ | mTNFa/ | mNGAL/ | mHO-1/ | Plasma BUN | Plasma Creatinine |
|---|---|---|---|---|---|---|
| SEQ ID NO | GAPDH | GAPDH | GAPDH | GAPDH | (mg/dL) | (mg/dL) |
| SEQ ID NO: 5 | 0.79 | 2.55 | 1.76 | 0.62 | 23 | 0.23 |
| SEQ ID NO: 54 | 0.74 | 2.42 | 1.58 | 0.65 | 26 | 0.26 |
| SEQ ID NO: 46 | 0.64 | 2.87 | 1.39 | 0.58 | 22 | 0.29 |
| PBS Control | 0.72 | 2.59 | 1.52 | 0.60 | 20 | 0.32 |

None of the above tested peptides induced a notable increase in any of the three measured mRNA and was consistent with a lack of nephrotoxicity. BUN and plasma creatinine concentrations remained normal. All measured values in mice were well below the upper limits of normal mice, which are BUN <30 and creatinine <0.6 mg/dL.

### Example 15

### Fluorescent Peptide Delivery to Kidneys

This example illustrates fluorescent peptide delivery to kidneys. Peptides were labeled by reaction with NHS esters of Cy5.5 or AlexaFluor 647 (AF647). For comparison, the free AF647 fluorophore NHS esters were hydrolyzed to produce unreactive Cy5.5-COOH and AF647-COOH. A dose of 10 nmol of dye-labeled peptide or dye alone was administered intravenously and fluorescence was measured at 3 hours, 24 hours, and 48 hours after administration. At each time point, mice were frozen and sectioned for whole body fluorescence analysis, which was performed by scanning the sections on the Odyssey 2.1 at 84 um resolution using the 700 channel.

TABLE 5 shows quantification of fluorescence signal of SEQ ID NO: 55 conjugated to Cy5.5 (SEQ ID NO: 55-Cy5.5) or SEQ ID NO: 55 conjugated to AlexaFluor 647 (SEQ ID NO: 55-AF647) was compared to free fluorophore Cy5.5 or AF647, respectively. Average and standard deviation are presented from two mice per group.

TABLE 5
Fluorescence Signal in Kidneys

| SEQ ID NO | 3 hours | 24 hours | 48 hours |
|---|---|---|---|
| SEQ ID NO: | Saturated Signal | Saturated Signal | 648 ± 181 |
| 55-Cy5.5 | (>3200) | (>5200) | |
| Cy5.5 | 416 ± 170 | 88 ± 8 | 58 ± 11 |
| SEQ ID NO: | 1904 ± 309 | 2286 ± 775 | 524 ± 98 |
| 55-AF647 | | | |
| AF647 | 349 ± 100 | 215 ± 0.07 | 125 ± 48 |

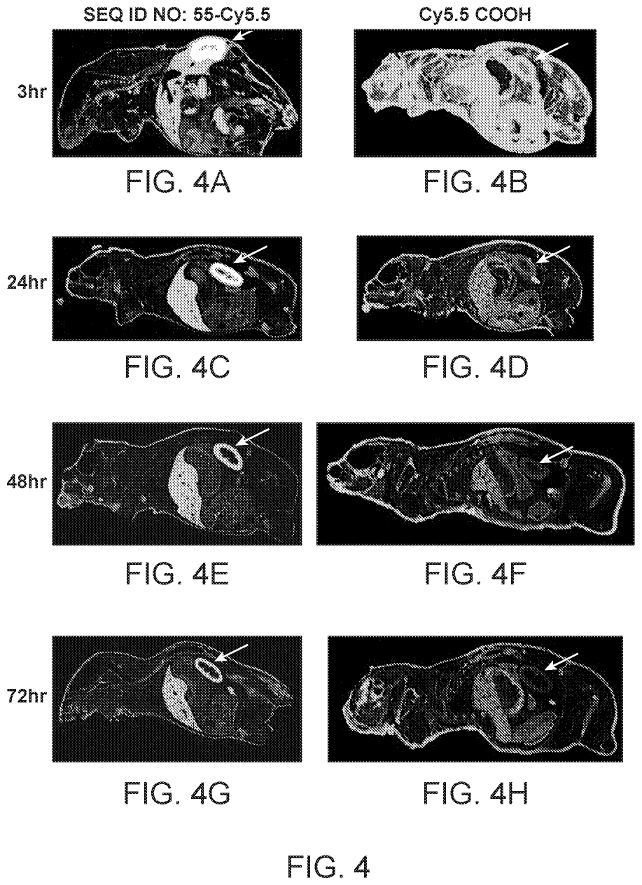

FIG. 4 shows whole body fluorescence images of mice after administration of SEQ ID NO: 55 conjugated to Cy5.5 (SEQ ID NO: 55-Cy5.5) (left) versus after administration of free Cy5.5-COOH alone (right). FIG. 4A shows a whole body fluorescence image of a mouse 3 hours after administration of 10 nmol SEQ ID NO: 55-Cy5.5. The arrow indicates the position and fluorescence signal in the kidney. FIG. 4B shows a whole body fluorescence image of a mouse 3 hours after administration of 10 nmol Cy5.5-COOH. The arrow indicates the position and fluorescence signal in the kidney. FIG. 4C shows a whole body fluorescence image of a mouse after 24 hours after administration of 10 nmol SEQ ID NO: 55-Cy5.5. The arrow indicates the position and fluorescence signal in the kidney. FIG. 4D shows a whole body fluorescence image of a mouse 24 hours after administration of 10 nmol Cy5.5-COOH. The arrow indicates the position and fluorescence signal in the kidney. FIG. 4E shows a whole body fluorescence image of a mouse 48 hours after administration of 10 nmol SEQ ID NO: 55-Cy5.5. The arrow indicates the position and fluorescence signal in the kidney. FIG. 4F shows a whole body fluorescence image of a mouse 48 hours after administration of 10 nmol Cy5.5-COOH. The arrow indicates the position and fluorescence signal in the kidney. FIG. 4G shows a whole body fluorescence image of a mouse 72 hours after administration of 10 nmol SEQ ID NO: 55-Cy5.5. The arrow indicates the position and fluorescence signal in the kidney. FIG. 4H shows a whole body fluorescence image of a mouse 72 hours after administration of 10 nmol Cy5.5-COOH. The arrow indicates the position and fluorescence signal in the kidney.

These results show that the peptide can deliver the conjugated molecules (Cy5.5 or AlexaFluor 647) to the kidney, resulting in accumulation of the dye in the kidneys and extended residence/AUC of the dye in the kidney; whereas dosage of the dye alone results in nonspecific distribution to many compartments of the body and reduced residence/AUC in the kidney.

### Example 16

### Whole Body Autoradiography

This example illustrates accumulation of peptides of the present disclosure in kidneys measured by whole body autoradiography. Peptides of the present disclosure were radiolabeled as described in EXAMPLE 2. 100 nmol of ¹⁴C labeled peptides were administered intravenously in mice. Radiolabeled peptide signal was quantified in the renal cortex and blood from the ventricle in 2-3 sections per mouse in 2 mice total. Whole body autoradiography analysis was performed with AIDA. Mice were euthanized at 3 hours and 24 hours post-administration and quantified signal is presented in TABLE 6.

TABLE 6
Radiolabeled Peptide Signal in Kidneys

| | Pixel density- | Signal ratio | Pixel density- | Signal ratio |
|---|---|---|---|---|
| | Bkg/area | Kidney:Blood | Bkg/area | Kidney:Blood |
| SEQ ID NO: | (3 hours) | (3 hours) | (24 hours) | (24 hours) |
| SEQ ID NO: 5 | 1.40E+07 | 27.2 | 1.81E+06 | 6.4 |
| SEQ ID NO: 54 | 1.48E+06 | 0.8 | 1.95E+06 | 7.2 |
| SEQ ID NO: 55 | 2.73E+07 | 42.2 | 2.25E+06 | 10.7 |
| SEQ ID NO: 46 | 8.83E+05 | 4.7 | 1.49E+06 | 6 |
| SEQ ID NO: 56 | 1.48E+07 | 29.9 | 2.30E+06 | 7.5 |
| SEQ ID NO: 57 | 4.63E+06 | 8 | 1.03E+06 | 6.4 |
| SEQ ID NO: 58 | 7.73E+06 | 9.9 | 1.30E+06 | 4.1 |
| SEQ ID NO: 59 | 3.47E+07 | 31.2 | 2.13E+06 | 6.1 |

The above data demonstrate that there is uniqueness among peptides with regard to accumulation and retention in the kidney, in which the peptides were localized for a longer period of time in the kidney as compared to other low molecular weight proteins, and which may be due to variation in biochemical properties, protease resistance, hydrophobicity, or charge.

SEQ ID NO: 55 and SEQ ID NO: 119 (GSGVPINVRSRGSRDSLDPSRRAGMRFGRSINSRSHSTP), a linearized version of SEQ ID NO: 55, were radiolabeled with ¹⁴C as described in EXAMPLE 2 and administered intravenously in mice at a dose of 100 nmol. Radiolabeled peptide signal was quantified in the renal cortex and blood from the ventricle in 2-3 sections per mouse in 2 mice total. Whole body autoradiography analysis was performed with AIDA. Mice were euthanized at 3 hours and 24 hours post-administration and quantified signal is presented in TABLE 7.

TABLE 7
Radiolabeled Peptide Signal in Knottin and Linearized Peptides

| | Pixel Density- | Signal ratio | Pixel Density- | Signal ratio |
|---|---|---|---|---|
| | Bkg/area | Kidney:Blood | Bkg/area | Kidney:Blood |
| SEQ ID NO | (3 hours) | (3 hours) | (24 hours) | (24 hours) |
| SEQ ID NO: 55 | 2.73E+07 | 42.4 | 2.25E+06 | 10.7 |
| SEQ ID NO: 119 | 1.71E+06 | 5.1 | 2.41E+06 | 6.9 |

Comparison between SEQ ID NO: 55 and SEQ ID NO: 119 at 3 hours post-administration demonstrated that the knottin structure was valuable in trafficking and accumulating peptides to the kidney. The increase of signal at 24 hours post-administration for SEQ ID NO: 119 possibly demonstrated that this linearized peptide was susceptible to degradation and the signal was a result of free ¹⁴C-Gly re-circulating through the kidney.

### Example 17

### Confocal Imaging of Kidneys

This example illustrates confocal imaging of kidneys from mice administered peptides of the present disclosure. A dose of 10 nmol of AlexFluor 647 (AF647) labeled peptide was administered intravenously in mice (2 per group). Mice were euthanized 20 hours post-peptide administration and kidneys were harvested and cut into 2 mm sections. Adjacent sections were scanned on an Odyssey instrument at 54 μm resolution in the 700 nm channel or imaged on a Zeiss laser scanning microscope (LSM) 780 confocal microscope at 6× and 20× magnification.

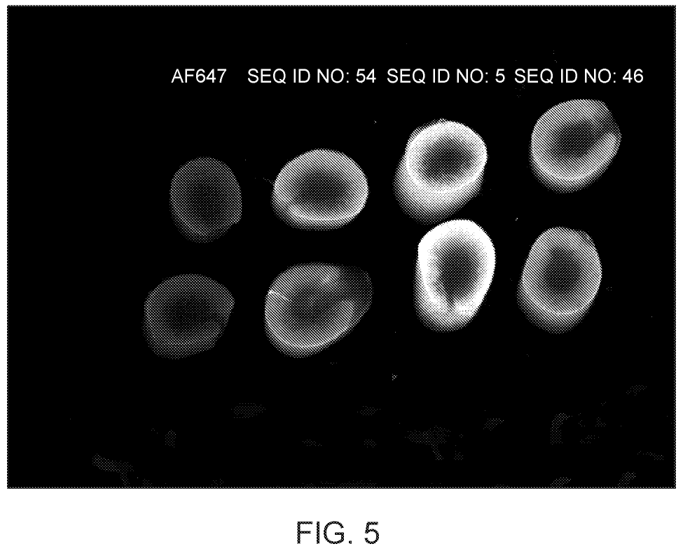

FIG. 5 shows fluorescence of kidney sections from mice, in which each mouse received 10 nmol free fluorophore (AF647), 10 nmol SEQ ID NO: 54 conjugated to AF647, 10 nmol SEQ ID NO: 5 conjugated to AF647, or 10 nmol SEQ ID NO: 46 conjugated to AF647. Each kidney was from an independent mouse (2 mice per group).

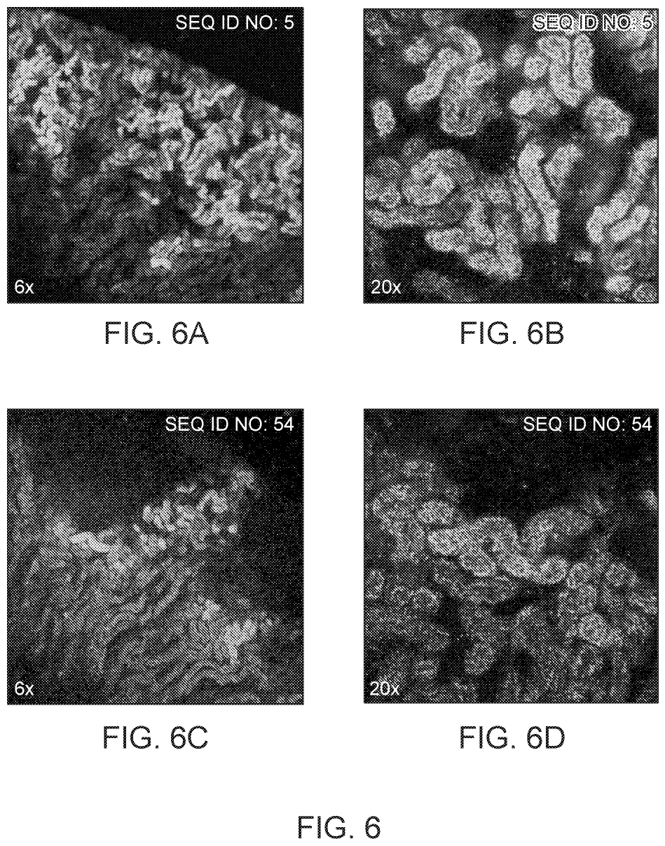

FIG. 6 shows SEQ ID NO: 5 conjugated to AF647 and SEQ ID NO: 54 conjugated to AF647 fluorescence signal in confocal images of the kidney cortex. FIG. 6A shows fluorescence signal of SEQ ID NO: 5 conjugated to AF647 in the kidney cortex 20 hours after of administration of 10 nmol of the peptide-dye conjugate at 6× magnification. FIG. 6B shows fluorescence signal of SEQ ID NO: 5 conjugated to AF647 in the kidney cortex 20 hours after administration of 10 nmol of the peptide-dye conjugate at 20× magnification. FIG. 6C shows fluorescence signal of SEQ ID NO: 5 conjugated to AF647 in the kidney cortex 20 hours after administration of 10 nmol of the peptide-dye conjugate at 6× magnification. FIG. 6D shows fluorescence signal of SEQ ID NO: 5 conjugated to AF647 in the kidney cortex 20 hours after of administration of 10 nmol of the peptide-dye conjugate at 20× magnification.

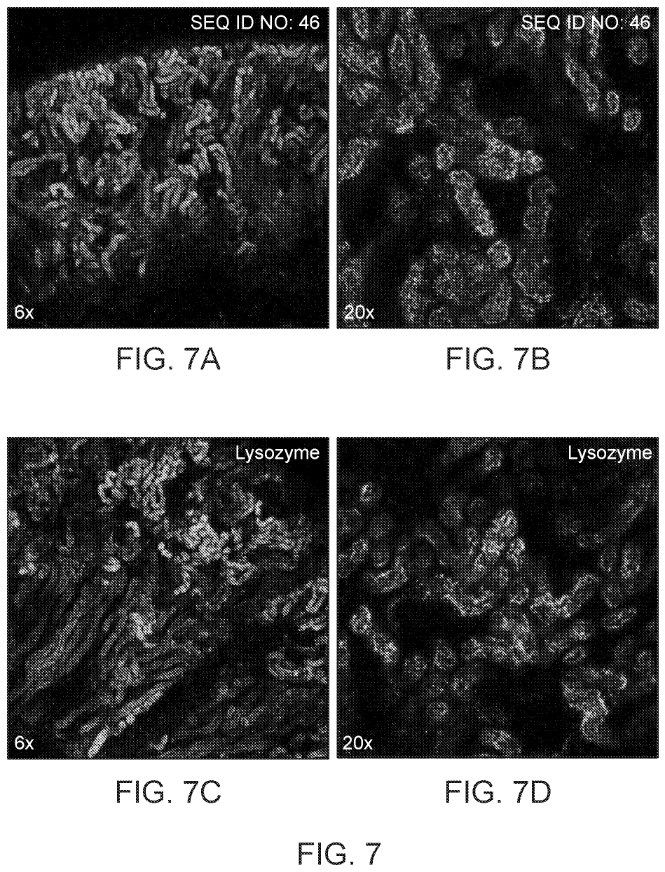

FIG. 7 shows SEQ ID NO: 46 conjugated to AF647 fluorescence signal in confocal images of the kidney cortex. FIG. 7A shows fluorescence signal of SEQ ID NO: 46 conjugated to AF647 in the kidney cortex 20 hours after administration of 10 nmol of the peptide-dye conjugate at 6× magnification. FIG. 7B shows fluorescence signal of SEQ ID NO: 46 conjugated to AF647 in the kidney cortex 20 hours after administration of 10 nmol of the peptide-dye conjugate at 20× magnification. FIG. 7C shows fluorescence signal in the kidney cortex 20 hours after administration of 10 nmol of a lysozyme-dye conjugate at 6× magnification. FIG. 7D shows fluorescence signal in the kidney cortex 20 hours after of administration of 10 nmol of a lysozyme-dye conjugate at 20× magnification.

Therefore, FIG. 5 shows that the peptides can accumulate the conjugated dye in the cortex of the kidney, and FIG. 6 and FIG. 7 show that the peptides can accumulate the conjugate dye in the proximal tubules in the kidney, as confirmed by the positive control lysozyme which has been shown to accumulate in the proximal tubules.

### Example 18

### Renal Accumulation and Urinary Excretion of Peptide

This example describes evaluation of renal accumulation and urinary excretion of peptides of this disclosure by liquid scintillation counting (LSC). Peptides were labeled with ¹⁴C as described in EXAMPLE 2. A dose of 100 nmol of radiolabeled peptides were administered intravenously in mice (3 per group) and mice were euthanized at 30 minutes, 1 hour, 2 hours, 4 hours, 8 hours, 24 hours, 48 hours, and 72 hours post administrationUrine, plasma, muscle, kidney, and the kidney cortex was harvested. Plasma (20 μl) and urine (5 μl) were analyzed for signal by LSC. Kidney and kidney cortex (muscle data not shown) were weighed and homogenized in 300 μl of Tris-based homogenization buffer with protease inhibitors using steel beads with a Qiagen TissuLyzer. Tissue homogenate (100 μl, uncentrifuged) was analyzed for signal by LSC.

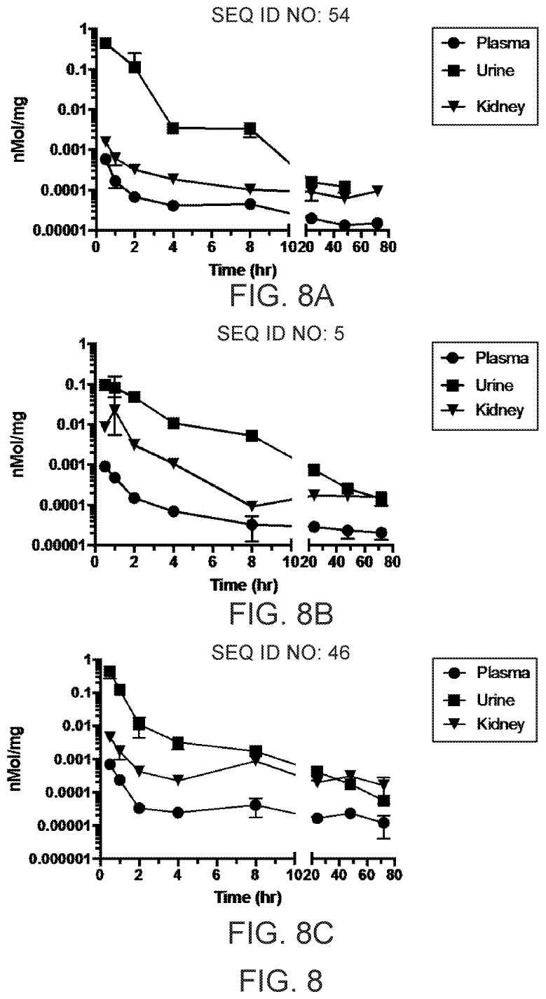

FIG. 8 shows the peptide concentration in plasma, urine, and kidney over time. FIG. 8A shows peptide concentration in plasma, urine, and kidney after intravenous administration of 50 nmol of radiolabeled SEQ ID NO: 54 peptide. FIG. 8B shows the peptide concentration in plasma, urine, and kidney after intravenous administration of 50 nmol of radiolabeled peptide of SEQ ID NO: 5. FIG. 8C shows the peptide concentration in plasma, urine, and kidney after intravenous administration of 50 nmol of a radiolabeled peptide of SEQ ID NO: 46.

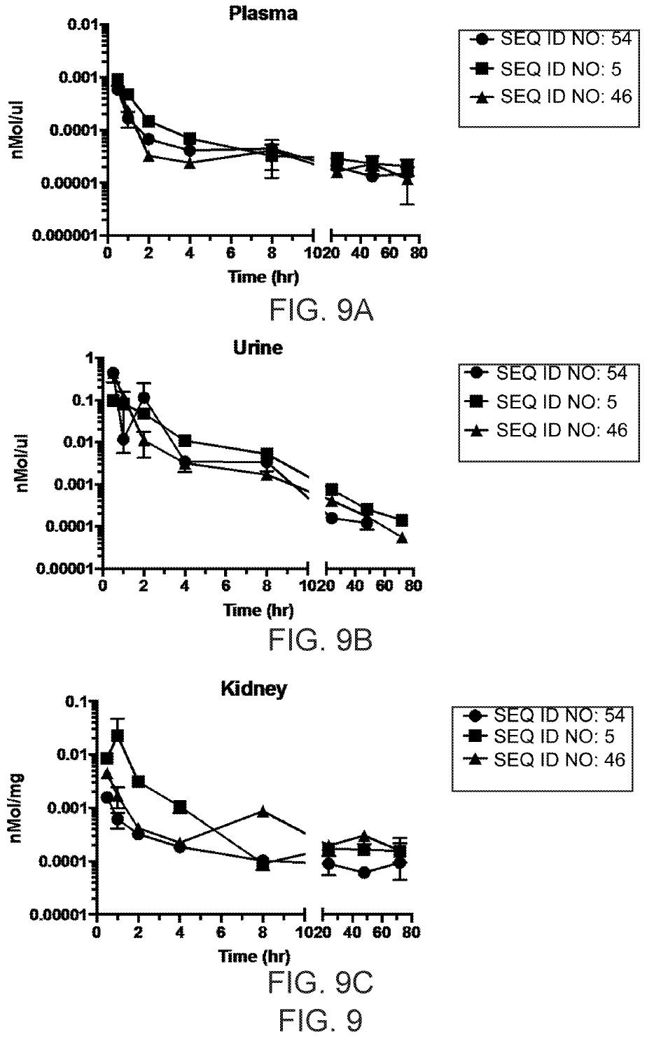

FIG. 9 shows the peptide concentration in plasma, urine, or kidney over time. FIG. 9A shows the peptide concentration in plasma after intravenous administration of 50 nmol radiolabeled SEQ ID NO: 54, 50 nmol radiolabeled SEQ ID NO: 5, or 50 nmol radiolabeled SEQ ID NO: 46. FIG. 9B shows the peptide concentration in urine after intravenous administration of 50 nmol radiolabeled SEQ ID NO: 54, 50 nmol radiolabeled SEQ ID NO: 5, or 50 nmol radiolabeled SEQ ID NO: 46 in urine. FIG. 9C shows the peptide concentration in kidney after intravenous administration of 50 nmol radiolabeled SEQ ID NO: 54, radiolabeled SEQ ID NO: 5, or radiolabeled SEQ ID NO: 46.

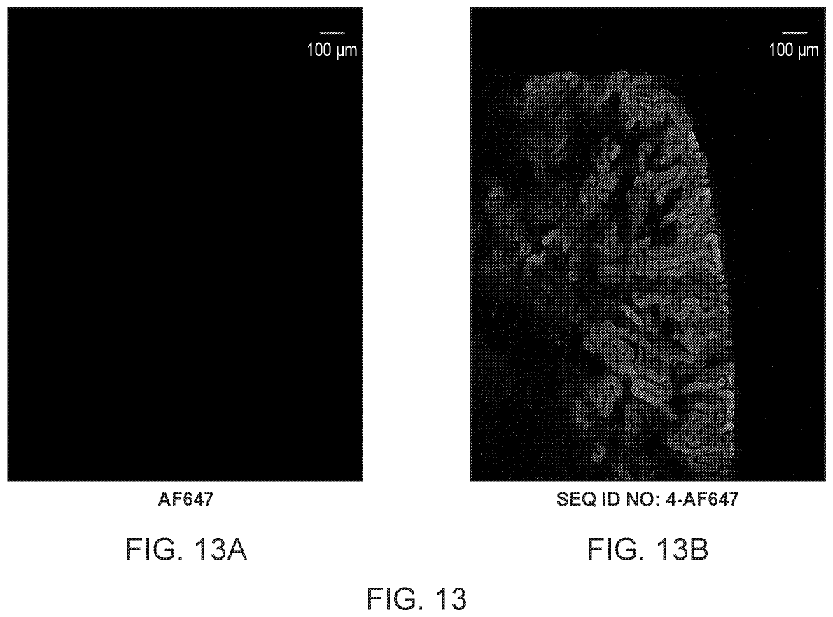

FIG. 13 shows fluorescence signal in the kidneys 30 minutes after adminstration of either nmol free AF647 fluorophore or 10 nmol SEQ ID NO: 4 conjugated to AF647 (SEQ ID NO: 4-AF647). Kidneys were isolated, sectioned, and imaged using a Zeiss confocal microscopy. FIG. 13A shows fluorescence signal from free AF647 fluorophore at 10× magnification. FIG. 13B shows fluorescence signal of SEQ ID NO: 4-AF647 at 40× magnification. This shows the peptide can deliver and accumulate dye when attached as a conjugate in the kidney proximal tubules whereas free dye was not seen accumulating.

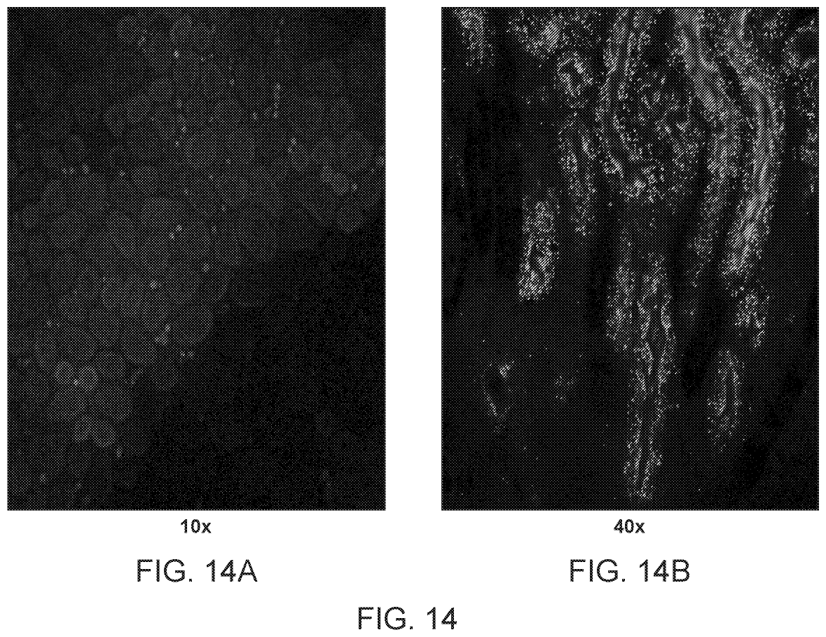

FIG. 14 shows fluorescence signal in the kidney 30 minutes after administration of 10 nmol SEQ ID NO: 46 conjugated to AF647 (SEQ ID NO: 46-AF647). Kidneys were isolated, sectioned, and imaged using a Zeiss confocal microscope. FIG. 14A shows fluorescence signal at 10× magnification. FIG. 14B shows fluorescence signal at 40× magnification.

### Example 19

### Competitive Renal Uptake Studies

This example describes competitive uptake studies of peptides of this disclosure in kidneys. Peptides of this disclosure were compared to known kidney homers (“competitors”) to assess the efficiency and strength of kidney targeting. Three competitors were tested against a peptide of SEQ ID NO: 4, and kidney uptake was quantified by fluorescence imaging of whole organs on a Spectrum IVIS imager.

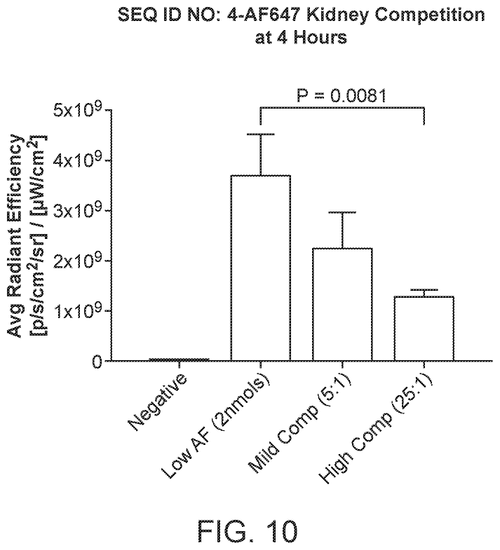

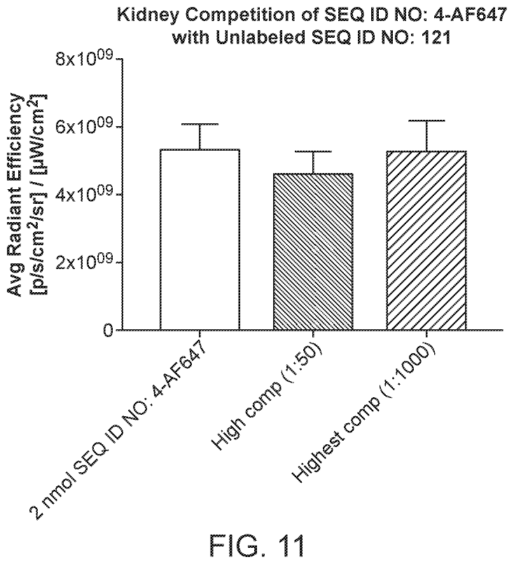

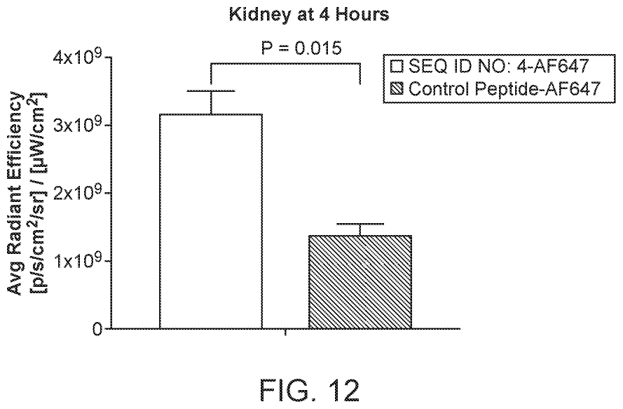

FIG. 10 shows competitive renal uptake between a peptide of SEQ ID NO: 4 conjugated to AlexaFluor647 (AF647) and an unlabeled SEQ ID NO: 4 peptide 4 hours after intravenous administration of 2 nmol of SEQ ID NO: 4-AF647 co-injected with either 0 nmol of SEQ ID NO: 4 peptide (“low AF”), 10 nmol of SEQ ID NO: 4 co-injected with 2 nmol of SEQ ID NO: 4-AF647 (5:1), or 50 nmol of SEQ ID NO: 4 co-injected with 2 nmol of SEQ ID NO: 4-AF647 (25:1). Kidneys from uninjected mice were used as a negative control. Fluorescence signal in each group was quantified to determine the average radiant efficiency in the kidneys from three mice per cohort. Data are shown as mean and error bars indicate standard deviation. A p-value of 0.0081 was calculated by a T-test, and the error bars indicate standard deviation. In this experiment, the unlabeled SEQ ID NO: 4 peptide competed with the SEQ ID NO: 4-AF647 as shown by decreased fluorescence and thus, decreased accumulation of the dye labeled peptide in the kidney. This indicates that SEQ ID NO: 4 peptide uptake was specific and saturable. In contrast, FIG. 11 shows no competitive renal uptake between a peptide of SEQ ID NO: 4 conjugated to AlexaFluor647 (AF647) and unlabeled KKEEEKKEEEKKEEEKK peptide (SEQ ID NO: 121, a known renal targeting peptide; see Bioconjug Chem. 2016 Apr. 20; 27(4):1050-7) 1 hour after intravenous administration of 2 nmol of a peptide of SEQ ID NO: 4-AF647, 2 nmol of a peptide of SEQ ID NO: 4-AF647 co-injected with 100 nmol of an unlabeled peptide of SEQ ID NO: 121 (1:50), or 2 nmol of peptide of SEQ ID NO: 4-AF647 co-injected with 2000 nmol of an unlabeled peptide of SEQ ID NO: 121 (1:1000). Fluorescence signal in each group was quantified to determine the average radiant efficiency in the kidneys from three mice per cohort. Data are shown as mean and error bars indicate standard deviation. Kidney uptake of a peptide of SEQ ID NO: 4-AF647 was not dampened by SEQ ID NO: 121 peptide even at the highest ratio of competitor. The SEQ ID NO: 121 peptide failed to compete with uptake of the peptide of SEQ ID NO: 4 in kidneys. Since SEQ ID NO: 121 has been hypothesized to bind to megalin, these results potentially indicate that SEQ ID NO: 4 peptide may accumulate in the proximal tubules by a different mechanism or receptor, or may bind to megalin more strongly than SEQ ID NO: 121 peptide. FIG. 12 also shows no competitive renal uptake between a peptide of SEQ ID NO: 4 conjugated to AlexaFluor647 (AF647) and a control peptide conjugated to AF647 (control peptide-AF647), 4 hours after intravenous administration of 10 nmol of a peptide of SEQ ID NO: 4-AF647 or 10 nmol of control peptide-AF647. Fluorescence signal in each group was quantified to determine the average radiant efficiency in the kidneys from three mice per cohort. Data are shown as mean and error bars indicate standard deviation. A p-value of 0.015 was calculated by a Student's unpaired t-test. The peptide of SEQ ID NO: 4 was taken up in the kidneys to a significantly higher extent than the control peptide.

### Example 20

### Peptide Stability

This example describes peptide stability in the presence of pepsin, trypsin, a reducing agent, or elevated temperature. Peptides were first suspended in 500u1 of ddH₂O to a stock concentration of 2 mg/ml. Reactions were prepared by adding 12.5k of peptide from the stock solution to a 10 mM solution of DTT in PBS and allowed to incubate at room temperature for 30 minutes. Other reactions were prepared with 12.5ₖ peptide and 5 μg trypsin in 25 mM Tris/75 mM NaCl buffer (pH 7.0) and incubated for 30 minutes at 37.5° C. These reactions were then quenched with 5 μg of soybean trypsin inhibitor (I) and, in some cases, 10 mM dithiothreitol (DTT). Other reactions were prepared with 12.5 μg peptide and 20 μg pepsin in pH 1.05, 2% (w/v) sodium chloride in 0.7% (v/v) hydrochloric acid and incubated for 30 minutes at 37.5° C. Temperature stability was tested by incubating peptides in non-reducing conditions at 70° C. for 1 hour. Stability was evaluated by RP-HPLC.

TABLE 8 shows a summary of peptides of this disclosure and their stability.

TABLE 8
Peptide Stability

| SEQ ID NO | Trypsin | Pepsin | Temperature (70° C.) | Reduction |
|---|---|---|---|---|
| 1 | Partially Resistant | Not Tested | Not Tested | Not Resistant |
| 5 | Resistant | Resistant | Not Tested | Resistant |
| 7 | Partially Resistant | Not Tested | Resistant | Not Resistant |
| 8 | Partially Resistant | Not Tested | Resistant | Not Resistant |
| 9 | Partially Resistant | Not Tested | Not Tested | Not Resistant |
| 10 | Not Resistant | Resistant | Resistant | Not Resistant |
| 11 | Resistant | Resistant | Not Tested | Resistant |
| 12 | Not Resistant | Not Tested | Not Tested | Partially Resistant |
| 14 | Not Resistant | Not Resistant | Resistant | Not Resistant |
| 16 | Not Resistant | Not Resistant | Resistant | Not Resistant |
| 17 | Partially Resistant | Not Tested | Not Tested | Not Resistant |
| 18 | Partially Resistant | Not Tested | Not Tested | Partially Resistant |
| 19 | Partially Resistant | Not Tested | Not Tested | Not Resistant |
| 20 | Partially Resistant | Not Tested | Not Tested | Resistant |
| 21 | Partially Resistant | Not Tested | Not Tested | Not Resistant |
| 22 | Partially Resistant | Not Tested | Not Tested | Not Resistant |
| 23 | Partially Resistant | Not Tested | Not Tested | Not Resistant |
| 24 | Partially Resistant | Not Tested | Not Tested | Resistant |
| 25 | Partially Resistant | Not Tested | Not Tested | Not Resistant |
| 26 | Partially Resistant | Not Tested | Not Tested | Not Resistant |
| 27 | Not Resistant | Not Resistant | Resistant | Not Resistant |
| 28 | Not Resistant | Not Tested | Not Tested | Partially Resistant |
| 29 | Partially Resistant | Not Tested | Not Tested | Not Resistant |
| 30 | Partially Resistant | Not Tested | Not Tested | Not Resistant |
| 31 | Partially Resistant | Not Tested | Not Tested | Not Resistant |
| 32 | Not Resistant | Resistant | Resistant | Not Resistant |
| 33 | Not Resistant | Resistant | Resistant | Not Resistant |
| 34 | Partially Resistant | Not Tested | Not Tested | Not Resistant |
| 35 | Partially Resistant | Not Tested | Not Tested | Not Resistant |
| 36 | Not Resistant | Resistant | Resistant | Not Resistant |
| 37 | Partially Resistant | Not Tested | Not Tested | Not Resistant |
| 38 | Not Resistant | Resistant | Resistant | Not Resistant |
| 39 | Partially Resistant | Not Tested | Not Tested | Not Resistant |
| 40 | Partially Resistant | Not Tested | Not Tested | Not Resistant |

### Example 21

### Preclinical Testing of Competitive Inhibition of Toxic Protein Uptake by Kidneys

This example illustrates preclinical validation in mice of competitive inhibition of toxic protein uptake by kidneys. Myoglobin is a toxic protein, which can accumulate in proximal tubules via megalin-mediated endocytosis. Peptides of this disclosure, which are injected in a subject at the time of kidney myoglobin exposure, will compete for megalin-mediated uptake.

A subject is injected intramuscularly with glycerol, leading to muscle injury with myoglobin release (also referred to herein as a “myoglobin challenge”). The subject in preclinical testing is a mouse. At the time of myoglobin injection, the subject is intravenously administered a peptide of this disclosure at one of a range of doses (0.1-2 mg/mouse) or saline as a negative control. Four hours after administration, the degree of myoglobin uptake by the kidney is tested using a spectrophotometric assay. The severity of myoglobin injury is assessed by testing for siderocalin mRNA (a biomarker of this process) upregulation.

Increasing the dose of the administered peptide of this disclosure causes a reciprocal decrease in myoglobin uptake in the kidney. Treatment of a subject with peptides of this disclosure results in dose-dependent blunting of siderocalin mRNA induction. In negative control subjects, which do not receive a peptide of this disclosure, glycerol injection causes an approximate 10-fold increase in siderocalin mRNA expression.

### Example 22

### Preclinical Testing of Alleviation of Renal Inflammation

This example illustrates preclinical validation in a subject of the alleviation of renal inflammation following endotoxin injection. A peptide of the present disclosure is conjugated to dexamethasone as described in EXAMPLE 11. The subject in preclinical testing is a mouse. Mice are injected intravenously with E. Coli endotoxin at 1 mg/kg to induce renal inflammation and co-injected intravenously either with saline as a negative control or with increasing doses of a peptide of this disclosure (0.1-2 mg/mouse). Four hours post-administration, severity of renal inflammation is assessed by measuring inflammatory mediator mRNAs, such as TNFa and monocyte chemoattractant protein (MCP)-1.

Co-injection of peptides of this disclosure causes dose-dependent blunting of mRNA upregulation. In negative control subjects, which do not receive a peptide of this disclosure, endotoxin injections induces an approximate 5-fold increase in TNFa and MCP-1 mRNA expression within 4 hours of endotoxin injection.

### Example 23

### Peptide Detectable Agent Conjugates

This example describes the dye labeling of peptides. A peptide of the disclosure is expressed recombinantly or chemically synthesized, and then the N-terminus of the peptide is conjugated to an detectable agent via an NHS ester using DCC or EDC to produce a peptide-detectable agent conjugate. The detectable agent is the fluorophore dye is a cyanine dye, such as Cy5.5 or an Alexa fluorophore, such as Alexa647.

The peptide detectable agent conjugates are administered to a subject. The subject can be a human or a non-human animal. After administration, the peptide detectable agent conjugates home to the kidneys. The subject, or a biopsy from the subject, is imaged to visualize localization of the peptide detectable agent conjugates to the kidney. In some aspects, diagnosis of renal disorders is based on the visualization of the peptide detectable agent conjugates in kidneys after administration.

### Example 24

### Peptide Deferoxamine Conjugates

This example describes conjugation of peptides of this disclosure to deferoxamine, an iron chelator. A peptide of the disclosure is expressed recombinantly or chemically synthesized, and then the N-terminus of the peptide is conjugated to deferoxamine via an NHS ester using DCC or EDC to produce a peptide-deferoxamine conjugate. Alternatively, a peptide can be conjugated to a deferoxamine by common techniques known in the art, such those described in Bioconjugate Techniques by Greg T. Hermanson (2013).

The peptide-deferoxamine conjugates are administered to a subject. The subject can be a human or non-human animal. The subject can have a pre-existing condition, such as iron poisoning. After administration, the peptide-deferoxamine conjugates home to the kidneys. Peptide-deferoxamine conjugates are used to treat iron poising by enhancing elimination of iron in urine.

### Example 25

### Peptide Bardoxolone Conjugates

This example describes conjugation of peptides of this disclosure to bardoxolone, an Nrf2 pathway activator. A peptide of the disclosure is expressed recombinantly or chemically synthesized, and then the N-terminus of the peptide is conjugated to bardoxolone to produce a peptide-bardoxolone conjugate. Optionally, a hydrolytically labile ester linkage is used in the conjugation, such that free bardoxolone is released after delivery to the kidney and/or proximal tubule.

The peptide-bardoxolone conjugates are administered to a subject. The subject can be a human or non-human animal. Optionally, a higher ratio of bardoxolone is seen in the kidney versus in serum after administration of the peptide-bardoxolone conjugate than when bardoxolone is administered alone. The subject can have a pre-existing condition, such as a renal disease. After administration, the peptide-bardoxolone conjugates home to the kidneys. Peptide-bardoxolone conjugates is used to treat patients with renal disease.

### Example 26

### Peptide Enalapril Conjugates

This example describes conjugation of peptides of this disclosure to enalapril, an angiotensin-converting-enzyme (ACE). A peptide of the disclosure is expressed recombinantly or chemically synthesized, and then using a carboxylic acid to make an ester, the peptide is conjugated to enalapril to produce a peptide-enalapril conjugate.

The peptide-enalapril conjugates are administered to a subject. The subject can be a human or non-human animal. The subject can have a pre-existing condition, such as hypertension, diabetic nephropathy, or heart failure. After administration, the peptide-enalapril conjugates home to the kidneys. Peptides-enalapril conjugates are used to prevent loss in kidney function in subjects with one of the above pre-existing conditions.

### Example 27

### Peptide Glycine Polymer Conjugates

This example describes conjugation of peptides of this disclosure to glycine polymers. A peptide of the disclosure is expressed recombinantly or chemically synthesized, and then the N-terminus of the peptide is conjugated to glycine polymers to produce a peptide-glycine polymer conjugate.

The peptide-glycine polymers conjugates are administered to a subject. The subject can be a human or non-human animal. The subject can have a pre-existing condition, such as kidney disease. The peptide-glycine polymer conjugate is used as a cytoprotectant. After administration, the peptide-glycine polymers conjugates are homed to the kidneys. Peptide-glycine polymer conjugates are used to prevent loss in kidney function in a subject.

### Example 28

### Peptide Antioxidant Conjugates

This example describes conjugation of peptides of this disclosure to an antioxidant. A peptide of the disclosure is expressed recombinantly or chemically synthesized, and then the N-terminus of the peptide is conjugated to an antioxidant to produce a peptide-antioxidant conjugate. The antioxidant can be glutathione or N acetyl cysteine.

The peptide-antioxidant conjugates are administered to a subject. The subject can be a human or non-human animal. The subject can have a pre-existing condition, such as diabetic nephropathy or post-ischemic or nephrotoxic AKI. After administration, the peptide-antioxidant conjugates are homed to the kidneys. Peptide-antioxidant conjugates are used to prevent loss in kidney function and protect renal function in subjects with one of the above pre-existing conditions.

### Example 29

### Prophylaxis Against Acute Kidney Injury

This example describes prophylaxis against acute kidney injury (AKI) with the peptides of the present disclosure. A peptide of this disclosure is expressed recombinantly or chemically synthesized. In some cases, the peptide is subsequently conjugated to an active agent. The peptide or peptide-active agent conjugate is administered to a subject in need thereof. The subject is a human or non-human animal. The subject in need thereof is at risk for acute kidney injury as a result of cardiovascular surgery, radiocontrast nephropathy, or cisplatin/carboplatin use. The peptide or peptide-conjugate is delivered via intravenous administration. Upon administration, the peptide or peptide conjugate rapidly targets the kidneys, and is used as prophylaxis against AKI.

### Example 30

### Treatment of Established Acute Kidney Injury

This example describes treatment of acute kidney injury (AKI) with the peptides of the present disclosure. A peptide of this disclosure is expressed recombinantly or chemically synthesized. In some cases, the peptide is subsequently conjugated to an active agent. The peptide or peptide-active agent conjugate is administered to a subject in need thereof. The subject is a human or non-human animal. The subject in need thereof has ischemic renal injury, endotoxemia-induced AKI, or established nephrotoxic AKI. The peptide or peptide-conjugate is delivered via intravenous administration. Upon administration, the peptide or peptide conjugate rapidly targets the kidneys, and is used to treat AKI.

### Example 31

### Treatment of Diabetic Nephropathy

This example describes treatment of diabetic nephropathy with the peptides of the present disclosure. A peptide of this disclosure is expressed recombinantly or chemically synthesized. In some cases, the peptide is subsequently conjugated to an active agent. The peptide or peptide-active agent conjugate is administered to a subject in need thereof. The subject is a human or non-human animal. The subject in need thereof is diagnosed with diabetic nephropathy. The peptide or peptide-conjugate is delivered via intravenous administration. Upon administration, the peptide or peptide conjugate rapidly targets the kidneys, and is used to treat diabetic nephropathy.

### Example 32

### Treatment of Hypertensive Nephrosclerosis

This example describes treatment of hypertensive nephrosclerosis with the peptides of the present disclosure. A peptide of this disclosure is expressed recombinantly or chemically synthesized. In some cases, the peptide is subsequently conjugated to an active agent. The peptide or peptide-active agent conjugate is administered to a subject in need thereof. The subject is a human or non-human animal. The subject in need thereof is has hypertensive nephrosclerosis. The peptide or peptide-conjugate is delivered via intravenous administration. Upon administration, the peptide or peptide conjugate is rapidly targeted to the kidneys, and is used to treat hypertensive nephrosclerosis.

### Example 33

### Treatment of Chronic Glomerulonephritis

This example describes treatment of chronic glomerulonephritis with the peptides of the present disclosure. A peptide of this disclosure is expressed recombinantly or chemically synthesized. In some cases, the peptide is subsequently conjugated to an active agent. The peptide or peptide-active agent conjugate is administered to a subject in need thereof. The subject is a human or non-human animal. The subject in need thereof is diagnosed with idiopathic or secondary chronic glomerulonephritis. The peptide or peptide-conjugate is delivered via intravenous administration. Upon administration, the peptide or peptide conjugate rapidly targets the kidneys, and is used to treat chronic glomerulonephritis.

### Example 34

### Treatment of Hereditary Nephropathy

This example describes treatment of hereditary nephropathy with the peptides of the present disclosure. A peptide of this disclosure is expressed recombinantly or chemically synthesized. In some cases, the peptide is subsequently conjugated to an active agent. The peptide or peptide-active agent conjugate is administered to a subject in need thereof. The subject is a human or non-human animal. The subject in need thereof is diagnosed with hereditary nephropathy, such as polycystic kidney disease or Alport's syndrome. The peptide or peptide-conjugate is delivered via intravenous administration. Upon administration, the peptide or peptide conjugate rapidly targets the kidneys, and is used to treat hereditary nephropathy.

### Example 35

### Treatment of Interstitial Nephritis

This example describes treatment of interstitial nephritis with the peptides of the present disclosure. A peptide of this disclosure is expressed recombinantly or chemically synthesized. In some cases, the peptide is subsequently conjugated to an active agent. The peptide or peptide-active agent conjugate is administered to a subject in need thereof. The subject is a human or non-human animal. The subject in need thereof is diagnosed with interstitial nephritis induced by drug use (e.g. Chinese herb induced nephropathy, NSAID induced nephropathy), multiple myeloma, or sarcoid. The peptide or peptide-conjugate is delivered via intravenous administration. Upon administration, the peptide or peptide conjugate rapidly targets the kidneys, and is used to treat interstitial nephritis.

### Example 36

### Use of Peptides in Renal Transplantation

This example describes the use of peptides of the present disclosure in renal transplantation. A peptide of this disclosure is expressed recombinantly or chemically synthesized. In some cases, the peptide is subsequently conjugated to an active agent. The peptide or peptide-active agent conjugate is administered to a subject in need thereof. The active agent is an anti-rejection drug such as prednisone, azathioprine, mycophenolate mofetil, mycophemolic acid, sirolimius, cyclosporine, or tacrolimus, and the subject is a human or non-human animal. A donor kidney is needed by the subject, which is treated with the peptide or peptide conjugate prior to transplantation. Alternatively, the subject is treated post-transplantation for delayed graft function, acute kidney rejection, or chronic rejection. For post-transplantation treatment, the peptide or peptide-conjugate is delivered via intravenous administration. Upon administration, the peptide or peptide conjugate rapidly targets the kidneys, and is used to treat post-transplantation kidney conditions.

### Example 37

### Use of Peptides to Treat Diabetes or High Blood Pressure

This example describes the use of peptides of the present disclosure to treat diabetes or high blood pressure. A peptide of this disclosure is expressed recombinantly or chemically synthesized. The peptide is administered to a subject in need thereof. Ion channels in the kidney (such as sodium channels or potassium channels) are modulated by the peptide, or the reuptake of glucose is blocked by the peptide. The subject is a human or non-human animal. The subject in need thereof is diagnosed with diabetes or high blood pressure. The peptide is delivered via intravenous administration. Upon administration, the peptide rapidly targets the kidneys and modulates sodium, potassium, or glucose transport in kidneys and is used to treat diabetes or high blood pressure.

### Example 38

### Use of Peptides to Prevent Renal Fibrosis

This example describes the use of peptides of the present disclosure to prevent renal fibrosis. A peptide of this disclosure is expressed recombinantly or chemically synthesized. The peptide is conjugated to a platelet derived growth factor (PDGF) inhibitor. The peptide-drug conjugate is administered to a subject in need thereof. The subject is a human or non-human animal. The subject in need thereof is at risk of renal fibrosis. The peptide is delivered via intravenous administration. Upon administration, the peptide rapidly targets the kidneys and prevents renal fibrosis.

### Example 39

### Oral Delivery to the Kidney

This example describes the oral delivery of peptides of the present disclosure. A peptide of this disclosure is expressed recombinantly or chemically synthesized. In some cases, the peptide is subsequently conjugated to an active agent. The peptide or peptide-active agent conjugate is administered orally to a subject in need thereof. The subject is a human or non-human animal. Upon administration, peptide or peptide-active agent rapidly targets the kidneys. Optionally, the peptide is formulated with agents to enhance oral delivery, such as permeation enhancers such as SNAC, 5-CNAC, sodium caprylate, an aromatic alcohol, EDTA, a sodium alkyl sulfate, or a citrate, or protease inhibitors. Some of the peptide is absorbed and traffics to the kidney.

### Example 40

### Peptide Validation

This example describes validation of peptides of the present disclosure made using the methods provided herein. Validation was carried out by evaluating expression using RP-HPLC and SDS-PAGE.

Various peptides were suspended in 500 μl of ddH₂O at a stock concentration of 2 mg/ml. This was then diluted in accord with the reaction conditions to prevent adverse buffering effects. Reactions were prepared with 12.5 μg peptide dissolved in solution and additionally suspended in a 10 mM solution of DTT. RP-HPLC was then run on samples using an Agilent 1260 HPLC equipped with a C-18 Poroshell 120B column. Sample were analyzed by a gradient method with a mobile phase of Solvent A (water with 0.1% TFA) and Solvent B (acetonitrile with 0.1% TFA). Solvent B was ramped up from 5%-45% of the mobile phase over a period of 10 minutes. Peptides were assessed for reduction by HPLC and by SDS-PAGE and compared to non-reduced peptide.

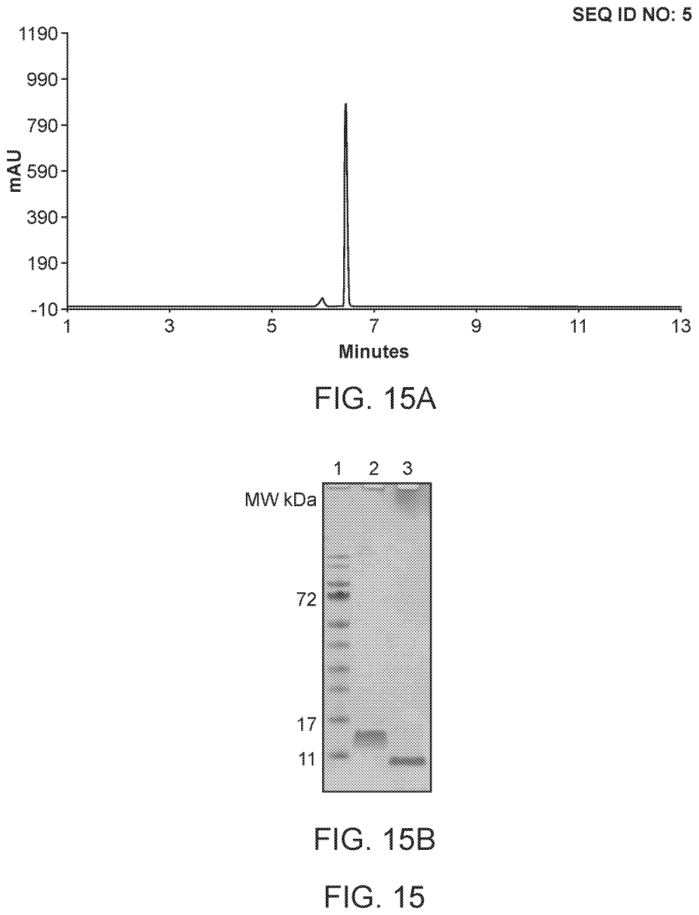

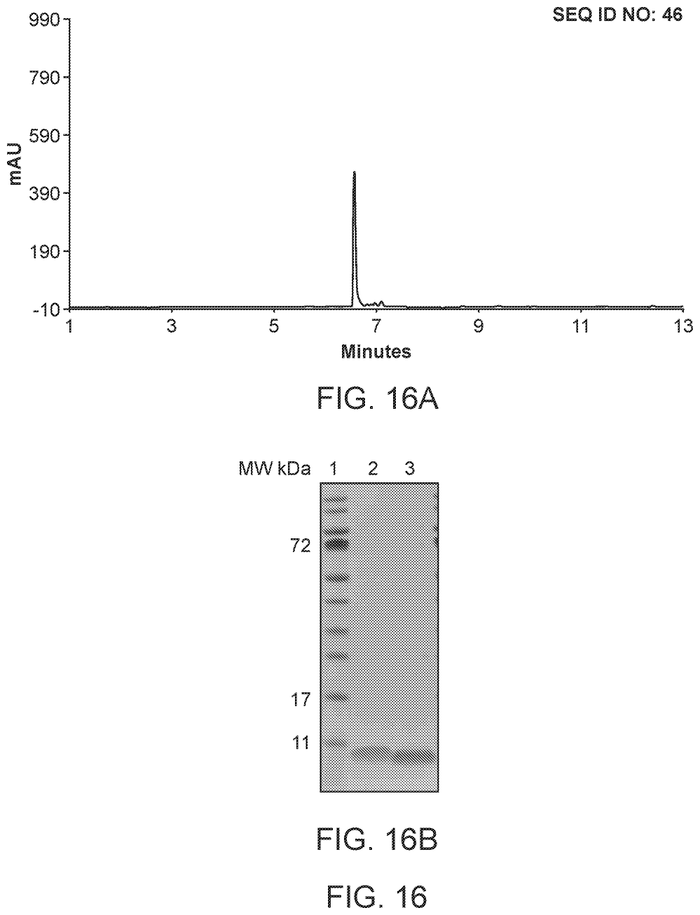

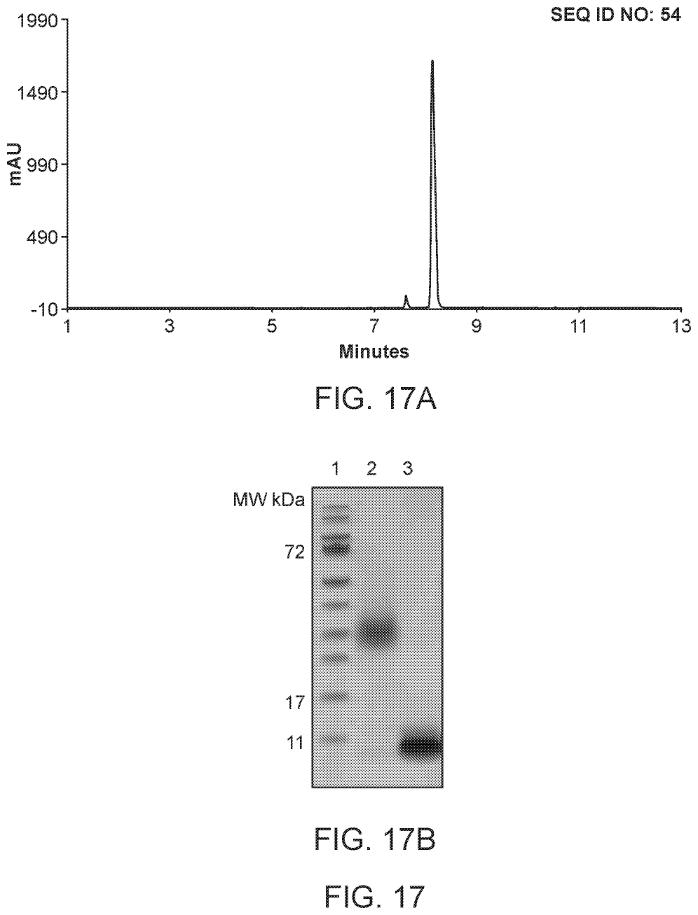

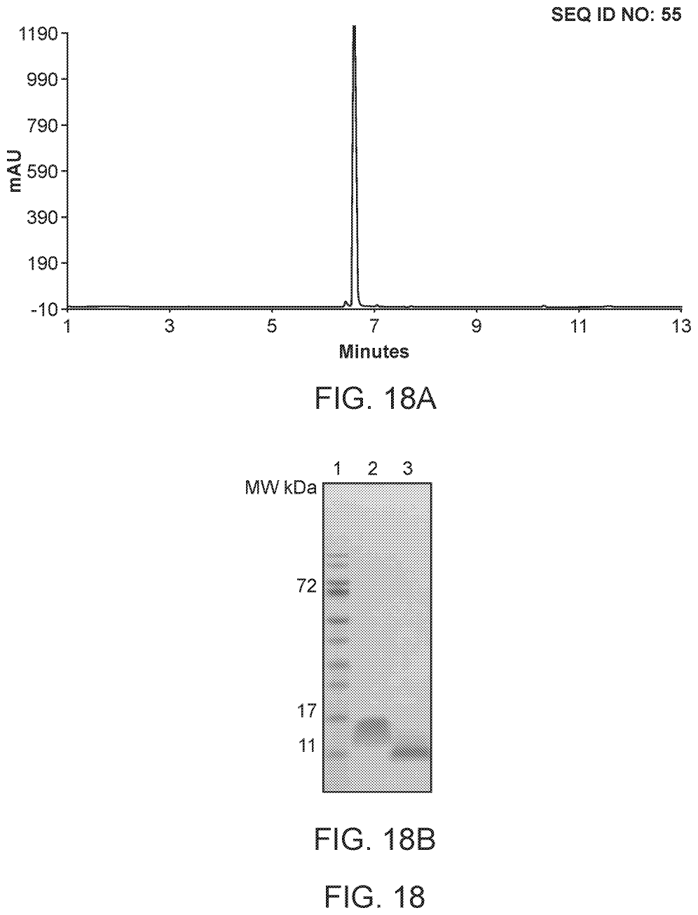

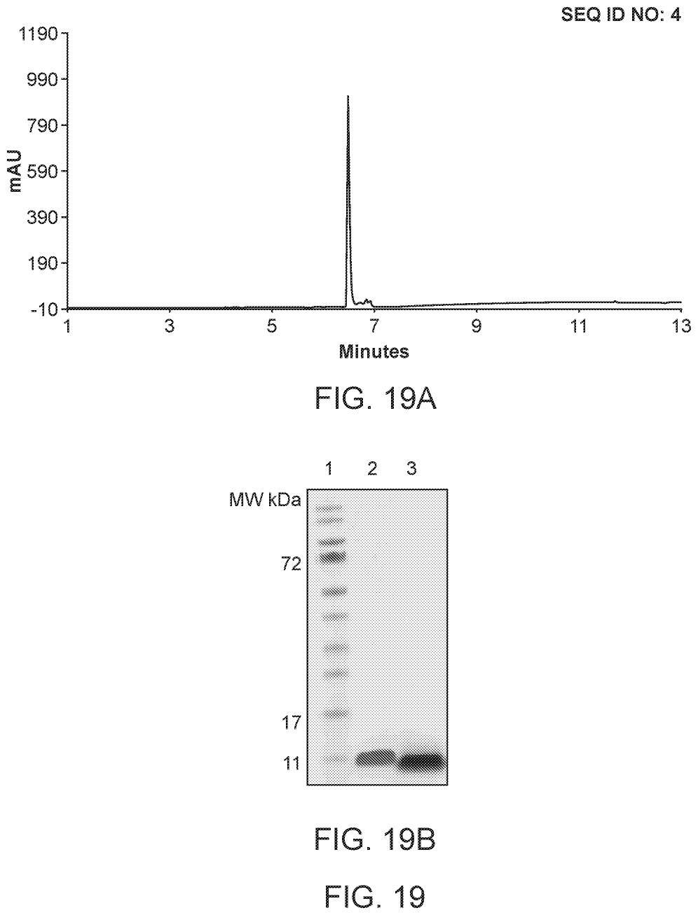

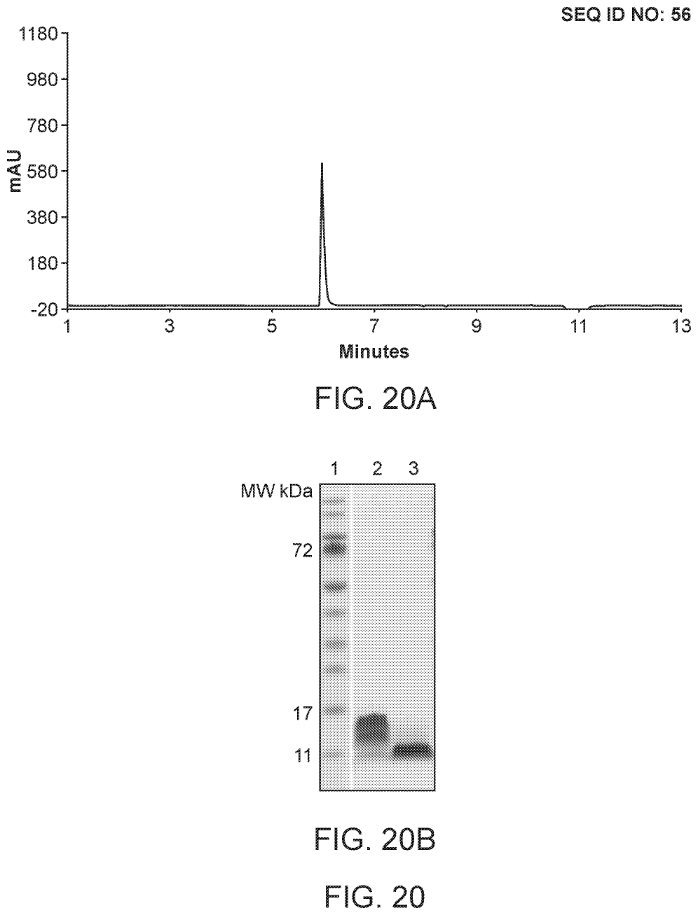

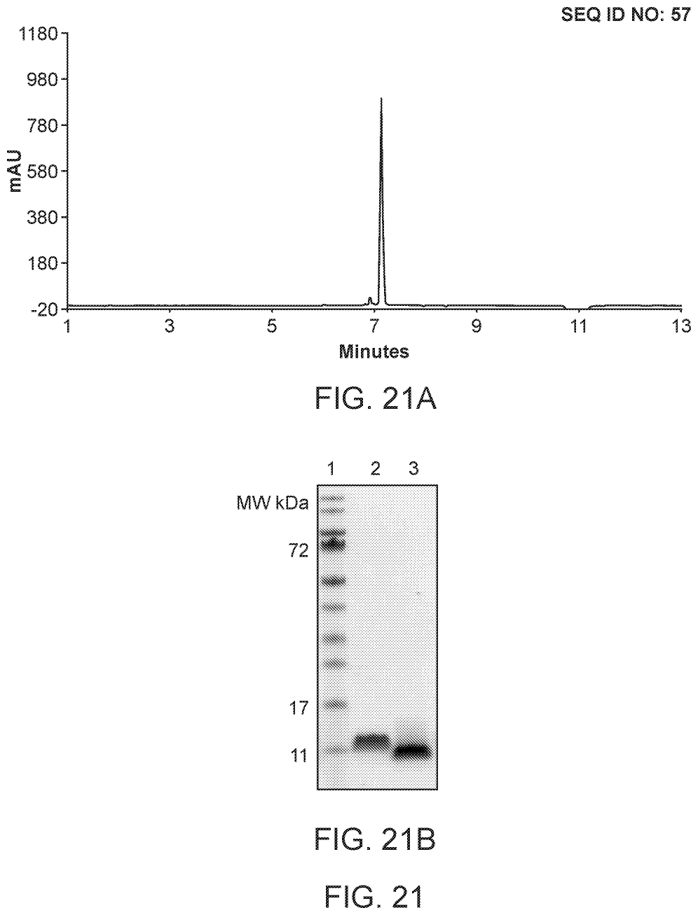

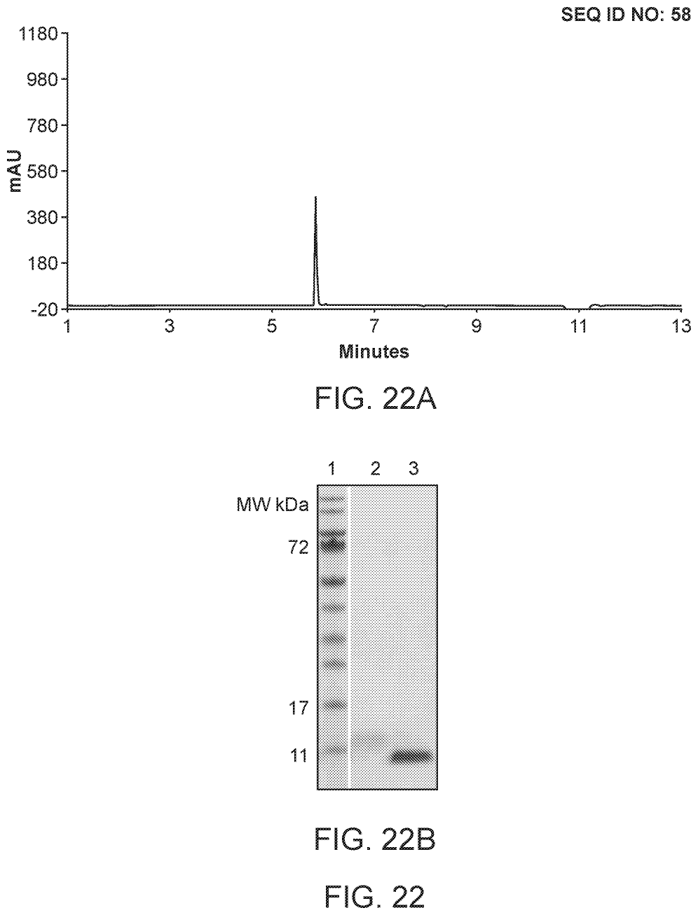

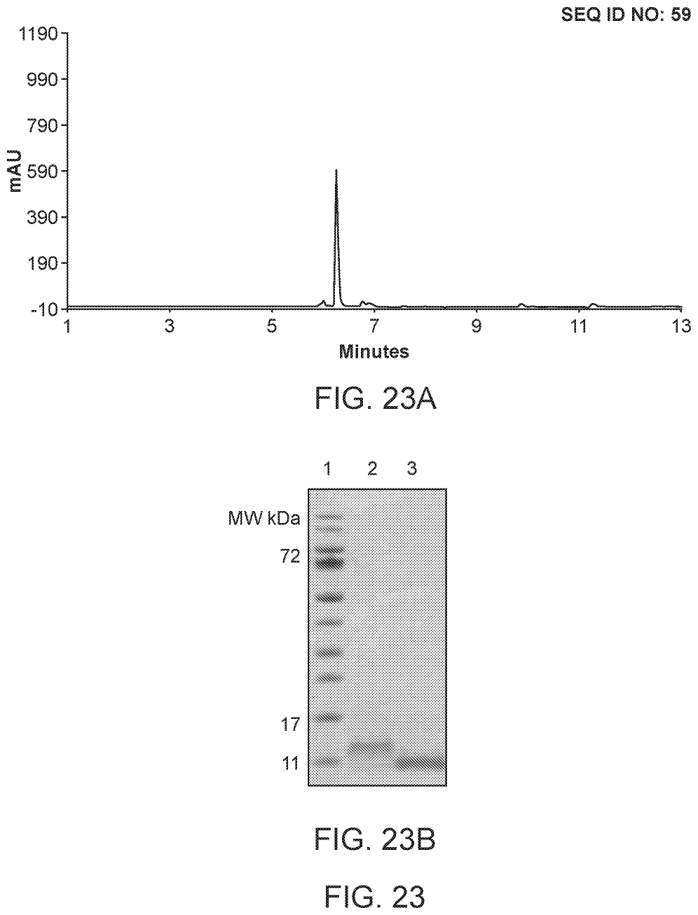

FIG. 15 shows stability results from a peptide of SEQ ID NO: 5. FIG. 15A shows the HPLC chromatogram of a non-reduced peptide of SEQ ID NO: 5. FIG. 15B shows an SDS-PAGE of a non-reduced (Lane 2) and reduced (Lane 3) of a peptide of SEQ ID NO: 5. FIG. 16 shows stability results from a peptide of SEQ ID NO: 46. FIG. 16A shows the HPLC chromatogram of a non-reduced peptide of SEQ ID NO: 46. FIG. 16B shows an SDS-PAGE of a non-reduced (Lane 2) and reduced (Lane 3) of a peptide of SEQ ID NO: 46. FIG. 17 shows stability results from a peptide of SEQ ID NO: 54. FIG. 17A shows the HPLC chromatogram of a non-reduced peptide of SEQ ID NO: 54. FIG. 17B shows an SDS-PAGE of a non-reduced (Lane 2) and reduced (Lane 3) of a peptide of SEQ ID NO: 54. FIG. 18 shows stability results from a peptide of SEQ ID NO: 55. FIG. 18A shows the HPLC chromatogram of a non-reduced peptide of SEQ ID NO: 55. FIG. 18B shows an SDS-PAGE of a non-reduced (Lane 2) and reduced (Lane 3) of a peptide of SEQ ID NO: 55. FIG. 19 shows stability results from a peptide of SEQ ID NO: 4. FIG. 19A shows the HPLC chromatogram of a non-reduced peptide of SEQ ID NO: 4. FIG. 19B shows an SDS-PAGE of a non-reduced (Lane 2) and reduced (Lane 3) of a peptide of SEQ ID NO: 4. FIG. 20 shows stability results from a peptide of SEQ ID NO: 56. FIG. 20A shows the HPLC chromatogram of a non-reduced peptide of SEQ ID NO: 56. FIG. 20B shows an SDS-PAGE of a non-reduced (Lane 2) and reduced (Lane 3) of a peptide of SEQ ID NO: 56. FIG. 21 shows stability results from a peptide of SEQ ID NO: 57. FIG. 21A shows the HPLC chromatogram of a non-reduced peptide of SEQ ID NO: 57. FIG. 21B shows an SDS-PAGE of a non-reduced (Lane 2) and reduced (Lane 3) of a peptide of SEQ ID NO: 57. FIG. 22 shows stability results from a peptide of SEQ ID NO: 58. FIG. 22A shows the HPLC chromatogram of a non-reduced peptide of SEQ ID NO: 58. FIG. 22B shows an SDS-PAGE of a non-reduced (Lane 2) and reduced (Lane 3) of a peptide of SEQ ID NO: 58. FIG. 23 shows stability results from a peptide of SEQ ID NO: 59. FIG. 23A shows the HPLC chromatogram of a non-reduced peptide of SEQ ID NO: 59. FIG. 23B shows an SDS-PAGE of a non-reduced (Lane 2) and reduced (Lane 3) of a peptide of SEQ ID NO: 59.

### Example 41

### Peptide Isoelectric Point

This example describes the isoelectric point of peptides of this disclosure. TABLE 9 shows the isoelectric point (pI) value for various peptides of this disclosure as calculated using the EMBOSS method. The pI refers to the isoelectric point and is the pH at which the net charge of the peptide is zero.

TABLE 9

| SEQ ID NO | pI |
|---|---|
| 1 | 10.409 |
| 2 | 9.408 |
| 3 | 10.608 |
| 4 | 8.389 |
| 5 | 9.235 |
| 6 | 8.211 |
| 7 | 7.756 |
| 8 | 8.59 |
| 9 | 9.689 |
| 10 | 6.103 |
| 11 | 9.23 |
| 12 | 9.22 |
| 13 | 10.288 |
| 14 | 8.121 |
| 15 | 9.1 |
| 16 | 8.603 |
| 17 | 8.885 |
| 18 | 9.69 |
| 19 | 9.122 |
| 20 | 9.167 |
| 21 | 8.202 |
| 22 | 8.634 |
| 23 | 8.115 |
| 24 | 8.626 |
| 25 | 8.869 |
| 26 | 9.179 |
| 27 | 8.866 |
| 28 | 8.403 |
| 29 | 7.638 |
| 30 | 7.751 |
| 31 | 8.412 |
| 32 | 8.624 |
| 33 | 8.866 |
| 34 | 8.37 |
| 35 | 7.962 |
| 36 | 9.171 |
| 37 | 8.86 |
| 38 | 8.623 |
| 39 | 6.975 |
| 40 | 5.659 |
| 41 | 7.608 |
| 42 | 7.606 |
| 43 | 8.211 |
| 44 | 9.123 |
| 45 | 12.132 |
| 46 | 8.116 |
| 47 | 8.631 |
| 48 | 8.631 |
| 49 | 8.631 |
| 50 | 6.117 |
| 51 | 8.384 |
| 52 | 8.116 |
| 53 | 9.097 |
| 54 | 6.103 |
| 55 | 9.292 |
| 56 | 10.39 |
| 57 | 9.292 |
| 58 | 8.895 |
| 59 | 10.409 |

While preferred embodiments of the present invention have been shown and described herein, it will be apparent to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-124. (canceled)

125. A method of treating a renal disorder in a subject in need thereof, the method comprising
administering to the subject in need thereof a peptide conjugate comprising a peptide and an active agent, wherein the active agent is a renal protective agent or a renal therapeutic agent;
homing the peptide conjugate to a renal tissue of the subject in need thereof; and
treating the renal disorder with the active agent.

126. The method of claim 125, wherein the peptide has at least 85% sequence identity to a peptide of any one of SEQ ID NO: 1-SEQ ID NO: 118 and SEQ ID NO: 122-SEQ ID NO: 180, or a fragment thereof.

127. The method of claim 125, wherein the active agent is selected from a glucocorticoid, an Nrf2 pathway activator, an ACE inhibitor, an antioxidant, an anti-rejection drug, an NSAID, an antibiotic, a senolytic, or a steroid.

128. The method of claim 125, wherein the active agent is selected from dexamethasone, triamcinolone acetonide, budesonide, mometasone, prednisone, azathioprine, mycophenolate mofetil, mycophemolic acid, sirolimius, cyclosporine, THR-184, or tacrolimus.

129. The method of claim 125, wherein the renal protective agent is selected from deferoxamine, hemin, iron chelate, porphyrin complex, or glycine polymers.

130. The method of claim 127, the Nrf2 pathway activator is bardoxolone.

131. The method of claim 127, wherein the ACE inhibitor is enalapril.

132. The method of claim 127, wherein the antioxidant is selected from glutathione or N-acetyl cysteine.

133. The method of claim 127, wherein the NSAID is ketorolac or ibuprofen.

134. The method of claim 127, wherein the antibiotic is selected from gentamicin, vancomycin, minocin, or mitomyclin.

135. The method of claim 125, wherein the renal disorder is selected from chronic kidney disease, diabetic nephropathy, renal fibrosis, lupus nephritis, acute kidney injury, chronic glomerulonephritis, interstitial nephritis, renal transplantation, high blood pressure, hypertensive nephrosclerosis, glomerulonephritis, polycystic kidney disease, or urinary obstruction.

136. The method of claim 125, wherein the peptide comprises at least 30 residues.

137. The method of claim 125, wherein the peptide comprises at least 4 cysteine residues.

138. The method of claim 125, wherein the peptide is coupled to a half-life modifying agent selected from a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin.

139. The method of claim 125, wherein the renal tissue is one or more of: a cortex region, a glomerulus, a proximal tubule, a medulla region, a descending tubule, an ascending tubule, a loop of Henle, or a Bowman's capsule of the subject.

140. The method of claim 125, the administering comprises inhalation, intranasally, orally, topically, intravenously, subcutaneously, intramuscularly administration, intraperitoneally, or any combination thereof.

141. The method of claim 125, the administering is once per day, week, or month, or once per two weeks, two months, or three months.

142. The method of claim 125, wherein the treating the renal disorder comprises protecting, preventing, prophylactically treating, arresting, reversing, ameliorating, reducing, or alleviating a symptom of the renal disorder.

143. The method of claim 142, wherein the protecting comprises protecting the renal tissue from injury.

144. The method of claim 125, wherein the treating the renal disorder comprises inducing ischemic preconditioning or acquired cytoresistance in the kidney of the subject.

145. The method of claim 125, wherein the peptide and the active agent are covalently or non-covalently linked, directly or via a linker.

146. A pharmaceutical composition comprising:
a peptide, wherein the peptide has at least 85% sequence identity to any one of SEQ ID NO: 1-SEQ ID NO: 118 and SEQ ID NO: 122-SEQ ID NO: 180, or a fragment thereof; and
a pharmaceutically acceptable excipient.

147. The composition of claim 146, wherein the peptide is further linked to an active agent, wherein the active agent comprises a renal protective agent or a renal therapeutic agent.

148. The composition of claim 147, wherein the active agent comprises a glucocorticoid, an Nrf2 pathway activator, an ACE inhibitor, an antioxidant, an anti-rejection drug, an NSAID, an antibiotic, a senolytic, THR-184, a steroid, dexamethasone, triamcinolone acetonide, budesonide, mometasone, prednisone, azathioprine, mycophenolate mofetil, mycophemolic acid, sirolimius, cyclosporine, tacrolimus, deferoxamine, hemin, iron chelate, porphyrin complex, glycine polymers, bardoxolone, enalapril, glutathione, N-acetyl cysteine, ketorolac, ibuprofen, gentamicin, vancomycin, minocin, or mitomyclin.

149. A peptide conjugate comprising
a peptide, wherein the peptide has at least 85% sequence identity to any one of SEQ ID NO: 1-SEQ ID NO: 118 and SEQ ID NO: 122-SEQ ID NO: 180, or a fragment thereof; and
an active agent, wherein the active agent comprises a renal protective agent or a renal therapeutic agent.

150. The peptide conjugate of claim 149, wherein the active agent comprises a glucocorticoid, an Nrf2 pathway activator, an ACE inhibitor, an antioxidant, an anti-rejection drug, an NSAID, an antibiotic, a senolytic, THR-184, a steroid, dexamethasone, triamcinolone acetonide, budesonide, mometasone, prednisone, azathioprine, mycophenolate mofetil, mycophemolic acid, sirolimius, cyclosporine, tacrolimus, deferoxamine, hemin, iron chelate, porphyrin complex, glycine polymers, bardoxolone, enalapril, glutathione, N-acetyl cysteine, ketorolac, ibuprofen, gentamicin, vancomycin, minocin, or mitomyclin.

151. A peptide comprising a sequence having at least 85% sequence identity to any one of SEQ ID NO: 1-SEQ ID NO: 118 and SEQ ID NO: 122-SEQ ID NO: 180, or a fragment thereof.

152. The peptide of claim 151, wherein the peptide is further linked to an active agent, wherein the active agent comprises a renal protective agent or a renal therapeutic agent.

153. The peptide of claim 152, wherein the active agent comprises a glucocorticoid, an Nrf2 pathway activator, an ACE inhibitor, an antioxidant, an anti-rejection drug, an NSAID, an antibiotic, a senolytic, THR-184, a steroid, dexamethasone, triamcinolone acetonide, budesonide, mometasone, prednisone, azathioprine, mycophenolate mofetil, mycophemolic acid, sirolimius, cyclosporine, tacrolimus, deferoxamine, hemin, iron chelate, porphyrin complex, glycine polymers, bardoxolone, enalapril, glutathione, N-acetyl cysteine, ketorolac, ibuprofen, gentamicin, vancomycin, minocin, or mitomyclin.


SEQUENCE LISTING (180 sequences)

SEQ ID NO: 1 - protein, 34 aa, Artificial Sequence
  Description of Artificial Sequence: Synthetic polypeptide
GSDCLPHLRRCRADNDCCGRRCRRRGTNAERRCR

SEQ ID NO: 2 - protein, 46 aa, Artificial Sequence
  Description of Artificial Sequence: Synthetic polypeptide
GSDCKYKFENWGACDGGTGTKVRQGTLKKARYNAQCQETIRVTKPC

SEQ ID NO: 3 - protein, 63 aa, Artificial Sequence
  Description of Artificial Sequence: Synthetic polypeptide
GSDCKYKFENWGACDGGTGTKVRQGTLKKARYNAQCQETIRVTKPCTPKTKAKAKAKKGKGKD

SEQ ID NO: 4 - protein, 36 aa, Artificial Sequence
  Description of Artificial Sequence: Synthetic polypeptide
GSSCEPGRTFRDRCNTCRCGADGRSAACTLRACPNQ

SEQ ID NO: 5 - protein, 39 aa, Artificial Sequence
  Description of Artificial Sequence: Synthetic polypeptide
GSQFTNVSCTTSRECWSVCQRLHNTSRGRCMNRRCRCYS

SEQ ID NO: 6 - protein, 38 aa, Artificial Sequence
  Description of Artificial Sequence: Synthetic polypeptide
GSMCMPCFTTDHQMARRCDDCCGGRGRGRCYGPQCLCR

SEQ ID NO: 7 - protein, 39 aa, Artificial Sequence
  Description of Artificial Sequence: Synthetic polypeptide
GSISIGIKCSPSIDLCEGQCRIRKYFTGYCSGDTCHCSG

SEQ ID NO: 8 - protein, 39 aa, Artificial Sequence
  Description of Artificial Sequence: Synthetic polypeptide
GSEVIRCSGSKQCYGPCKQQTGCTNSKCMNKVCKCYGCG

SEQ ID NO: 9 - protein, 37 aa, Artificial Sequence
  Description of Artificial Sequence: Synthetic polypeptide
GSSEKDCIKHLQRCRENKDCCSKKCSRRGTNPEKRCR

SEQ ID NO: 10 - protein, 36 aa, Artificial Sequence
  Description of Artificial Sequence: Synthetic polypeptide
GSSCAKPRENCNRMNILCCRGECVCPTFGDCFCYGD

SEQ ID NO: 11 - protein, 39 aa, Artificial Sequence
  Description of Artificial Sequence: Synthetic polypeptide
GSGVPINVKCRGSRDCLDPCKKAGMRFGKCINSKCHCTP

SEQ ID NO: 12 - protein, 39 aa, Artificial Sequence
  Description of Artificial Sequence: Synthetic polypeptide
GSVRIPVSCKHSGQCLKPCKDAGMRFGKCMNGKCDCTPK

SEQ ID NO: 13 - protein, 34 aa, Artificial Sequence
  Description of Artificial Sequence: Synthetic polypeptide
GSGIVCKVCKIICGMQGKKVNICKAPIKCKCKKG

SEQ ID NO: 14 - protein, 39 aa, Artificial Sequence
  Description of Artificial Sequence: Synthetic polypeptide
GSDCVRFWGKCSQTSDCCPHLACKSKWPRNICVWDGSVG

SEQ ID NO: 15 - protein, 35 aa, Artificial Sequence
  Description of Artificial Sequence: Synthetic polypeptide
GSAVCVYRTCDKDCKRRGYRSGKCINNACKCYPYG

SEQ ID NO: 16 - protein, 35 aa, Artificial Sequence
  Description of Artificial Sequence: Synthetic polypeptide
GSGCFGYKCDYYKGCCSGYVCSPTWKWCVRPGPGR

SEQ ID NO: 17 - protein, 40 aa, Artificial Sequence
  Description of Artificial Sequence: Synthetic polypeptide
GSQVQTNVKCQGGSCASVCRREIGVAAGKCINGKCVCYRN

SEQ ID NO: 18 - protein, 35 aa, Artificial Sequence
  Description of Artificial Sequence: Synthetic polypeptide
GSGDCLPHLKRCKENNDCCSKKCKRRGANPEKRCR

SEQ ID NO: 19 - protein, 37 aa, Artificial Sequence
  Description of Artificial Sequence: Synthetic polypeptide
GSNFKVEGACSKPCRKYCIDKGARNGKCINGRCHCYY

SEQ ID NO: 20 - protein, 40 aa, Artificial Sequence
  Description of Artificial Sequence: Synthetic polypeptide
GSQKILSNRCNNSSECIPHCIRIFGTRAAKCINRKCYCYP

SEQ ID NO: 21 - protein, 44 aa, Artificial Sequence
  Description of Artificial Sequence: Synthetic polypeptide
GSDRDSCIDKSRCSKYGYYQECQDCCKKAGHNGGTCMFFKCKCA

SEQ ID NO: 22 - protein, 34 aa, Artificial Sequence
  Description of Artificial Sequence: Synthetic polypeptide
GSAVCNLKRCQLSCRSLGLLGKCIGDKCECVKHG

SEQ ID NO: 23 - protein, 39 aa, Artificial Sequence
  Description of Artificial Sequence: Synthetic polypeptide
GSQFCGTNGKPCVNGQCCGALRCVVTYHYADGVCLKMNP

SEQ ID NO: 24 - protein, 38 aa, Artificial Sequence
  Description of Artificial Sequence: Synthetic polypeptide
GSRPTDIKCSASYQCFPVCKSRFGKTNGRCVNGLCDCF

SEQ ID NO: 25 - protein, 37 aa, Artificial Sequence
  Description of Artificial Sequence: Synthetic polypeptide
GSNCAGYMRECKEKLCCSGYVCSSRWKWCVLPAPWRR

SEQ ID NO: 26 - protein, 39 aa, Artificial Sequence
  Description of Artificial Sequence: Synthetic polypeptide
GSQFTDVKCTGSKQCWPVCKQMFGKPNGKCMNGKCRCYS

SEQ ID NO: 27 - protein, 40 aa, Artificial Sequence
  Description of Artificial Sequence: Synthetic polypeptide
GSQIDTNVKCSGSSKCVKICIDRYNTRGAKCINGRCTCYP

SEQ ID NO: 28 - protein, 40 aa, Artificial Sequence
  Description of Artificial Sequence: Synthetic polypeptide
GSAEIIRCSGTRECYAPCQKLTGCLNAKCMNKACKCYGCV

SEQ ID NO: 29 - protein, 38 aa, Artificial Sequence
  Description of Artificial Sequence: Synthetic polypeptide
GSSDYCSNDFCFFSCRRDRCARGDCENGKCVCKNCHLN

SEQ ID NO: 30 - protein, 41 aa, Artificial Sequence
  Description of Artificial Sequence: Synthetic polypeptide
GSCIGEGVPCDENDPRCCFGLVCLKPTLHGIWYKSYYCYKK

SEQ ID NO: 31 - protein, 33 aa, Artificial Sequence
  Description of Artificial Sequence: Synthetic polypeptide
GSSCAKPGEMCMRIKCCDGQCGCNRGTGRCFCK

SEQ ID NO: 32 - protein, 37 aa, Artificial Sequence
  Description of Artificial Sequence: Synthetic polypeptide
GSACLAEYQKCEGSTVPCCPGLSCSAGRFRKTKLCTK

SEQ ID NO: 33 - protein, 37 aa, Artificial Sequence
  Description of Artificial Sequence: Synthetic polypeptide
GSVVIGQRCYRSPDCYSACKKLVGKATGKCTNGRCDC

SEQ ID NO: 34 - protein, 34 aa, Artificial Sequence
  Description of Artificial Sequence: Synthetic polypeptide
GSACQFWSCNSSCISRGYRQGYCWGIQYKYCQCQ

SEQ ID NO: 35 - protein, 38 aa, Artificial Sequence
  Description of Artificial Sequence: Synthetic polypeptide
GSRCPPCFTTNPNMEADCRKCCGGRGYCASYQCICPGG

SEQ ID NO: 36 - protein, 39 aa, Artificial Sequence
  Description of Artificial Sequence: Synthetic polypeptide
GSVFINVKCRGSPECLPKCKEAIGKSAGKCMNGKCKCYP

SEQ ID NO: 37 - protein, 40 aa, Artificial Sequence
  Description of Artificial Sequence: Synthetic polypeptide
GSQVSTNKKCSNTSQCYKTCEKVVGVAAGKCMNGKCICYP

SEQ ID NO: 38 - protein, 38 aa, Artificial Sequence
  Description of Artificial Sequence: Synthetic polypeptide
GSECLEIFKACNPSNDQCCKSSKLVCSRKTRWCKYQIG

SEQ ID NO: 39 - protein, 56 aa, Artificial Sequence
  Description of Artificial Sequence: Synthetic polypeptide
GSQDKCKKVYENYPVSKCQLANQCNYDCKLDKHARSGECFYDEKRNLQCICDYCEY

SEQ ID NO: 40 - protein, 24 aa, Artificial Sequence
  Description of Artificial Sequence: Synthetic peptide
GSGHACYRNCWREGNDEETCKERC

SEQ ID NO: 41 - protein, 38 aa, Artificial Sequence
  Description of Artificial Sequence: Synthetic polypeptide
GSMCMPCFTTDTQMQERCDRCCGGGGRGRCWGPQCLCI

SEQ ID NO: 42 - protein, 38 aa, Artificial Sequence
  Description of Artificial Sequence: Synthetic polypeptide
GSMCMPCFTTEQRMAIICDDCCGGFGRGRCYGPQCLCR

SEQ ID NO: 43 - protein, 38 aa, Artificial Sequence
  Description of Artificial Sequence: Synthetic polypeptide
GSICIPCFTTDHQIARRCDDCCGGRGRGRCYGPQCICR

SEQ ID NO: 44 - protein, 44 aa, Artificial Sequence
  Description of Artificial Sequence: Synthetic polypeptide
GSRCQLQGFNCVVRSYGLPTIPCCRGLTCRSYFPGSTYGRCQRY

SEQ ID NO: 45 - protein, 40 aa, Artificial Sequence
  Description of Artificial Sequence: Synthetic polypeptide
GSSFGLCRLRRGFCARGRCRFPSIPIGRCSRFVQCCRRVW

SEQ ID NO: 46 - protein, 35 aa, Artificial Sequence
  Description of Artificial Sequence: Synthetic polypeptide
GSSCEPGTTFRDRCNTCRCGSDGRSAACTLRACPQ

SEQ ID NO: 47 - protein, 38 aa, Artificial Sequence
  Description of Artificial Sequence: Synthetic polypeptide
GSSCTPGTTFRDRCNTCRCSSNGRSAACTLRACPPGSY

SEQ ID NO: 48 - protein, 38 aa, Artificial Sequence
  Description of Artificial Sequence: Synthetic polypeptide
GSSCTPGTTFRNRCNTCRCGSNGRSASCTLMACPPGSY

SEQ ID NO: 49 - protein, 38 aa, Artificial Sequence
  Description of Artificial Sequence: Synthetic polypeptide
GSSCTPGATFRNRCNTCRCGSNGRSASCTLMACPPGSY

SEQ ID NO: 50 - protein, 33 aa, Artificial Sequence
  Description of Artificial Sequence: Synthetic polypeptide
GSSCQPGTTYQRGCNTCRCLEDGQTEACTLRLC

SEQ ID NO: 51 - protein, 38 aa, Artificial Sequence
  Description of Artificial Sequence: Synthetic polypeptide
GSSCTPGATYREGCNICRCRSDGRSGACTRRICPVDSN

SEQ ID NO: 52 - protein, 34 aa, Artificial Sequence
  Description of Artificial Sequence: Synthetic polypeptide
GSSCQPGTTFRRDCNTCVCNRDGTNAACTLRACL

SEQ ID NO: 53 - protein, 46 aa, Artificial Sequence
  Description of Artificial Sequence: Synthetic polypeptide
GGYSRCQLQGFNCVVRSYGLPTIPCCRGLTCRSYFPGSTYGRCQRY

SEQ ID NO: 54 - protein, 36 aa, Artificial Sequence
  Description of Artificial Sequence: Synthetic polypeptide
GSSCARPRENCNRMNILCCRGECVCPTFGDCFCYGD

SEQ ID NO: 55 - protein, 39 aa, Artificial Sequence
  Description of Artificial Sequence: Synthetic polypeptide
GSGVPINVRCRGSRDCLDPCRRAGMRFGRCINSRCHCTP

SEQ ID NO: 56 - protein, 37 aa, Artificial Sequence
  Description of Artificial Sequence: Synthetic polypeptide
GSSERDCIRHLQRCRENRDCCSRRCSRRGTNPERRCR

SEQ ID NO: 57 - protein, 39 aa, Artificial Sequence
  Description of Artificial Sequence: Synthetic polypeptide
GSVRIPVSCRHSGQCLRPCRDAGMRFGRCMNGRCDCTPR

SEQ ID NO: 58 - protein, 40 aa, Artificial Sequence
  Description of Artificial Sequence: Synthetic polypeptide
GSQVQTNVRCQGGSCASVCRREIGVAAGRCINGRCVCYRN

SEQ ID NO: 59 - protein, 35 aa, Artificial Sequence
  Description of Artificial Sequence: Synthetic polypeptide
GSGDCLPHLRRCRENNDCCSRRCRRRGANPERRCR

SEQ ID NO: 60 - protein, 32 aa, Artificial Sequence
  Description of Artificial Sequence: Synthetic polypeptide
DCLPHLRRCRADNDCCGRRCRRRGTNAERRCR

SEQ ID NO: 61 - protein, 44 aa, Artificial Sequence
  Description of Artificial Sequence: Synthetic polypeptide
DCKYKFENWGACDGGTGTKVRQGTLKKARYNAQCQETIRVTKPC

SEQ ID NO: 62 - protein, 61 aa, Artificial Sequence
  Description of Artificial Sequence: Synthetic polypeptide
DCKYKFENWGACDGGTGTKVRQGTLKKARYNAQCQETIRVTKPCTPKTKAKAKAKKGKGKD

SEQ ID NO: 63 - protein, 34 aa, Artificial Sequence
  Description of Artificial Sequence: Synthetic polypeptide
SCEPGRTFRDRCNTCRCGADGRSAACTLRACPNQ

SEQ ID NO: 64 - protein, 37 aa, Artificial Sequence
  Description of Artificial Sequence: Synthetic polypeptide
QFTNVSCTTSRECWSVCQRLHNTSRGRCMNRRCRCYS

SEQ ID NO: 65 - protein, 36 aa, Artificial Sequence
  Description of Artificial Sequence: Synthetic polypeptide
MCMPCFTTDHQMARRCDDCCGGRGRGRCYGPQCLCR

SEQ ID NO: 66 - protein, 37 aa, Artificial Sequence
  Description of Artificial Sequence: Synthetic polypeptide
ISIGIKCSPSIDLCEGQCRIRKYFTGYCSGDTCHCSG

SEQ ID NO: 67 - protein, 37 aa, Artificial Sequence
  Description of Artificial Sequence: Synthetic polypeptide
EVIRCSGSKQCYGPCKQQTGCTNSKCMNKVCKCYGCG

SEQ ID NO: 68 - protein, 35 aa, Artificial Sequence
  Description of Artificial Sequence: Synthetic polypeptide
SEKDCIKHLQRCRENKDCCSKKCSRRGTNPEKRCR

SEQ ID NO: 69 - protein, 34 aa, Artificial Sequence
  Description of Artificial Sequence: Synthetic polypeptide
SCAKPRENCNRMNILCCRGECVCPTFGDCFCYGD

SEQ ID NO: 70 - protein, 37 aa, Artificial Sequence
  Description of Artificial Sequence: Synthetic polypeptide
GVPINVKCRGSRDCLDPCKKAGMRFGKCINSKCHCTP

SEQ ID NO: 71 - protein, 37 aa, Artificial Sequence
  Description of Artificial Sequence: Synthetic polypeptide
VRIPVSCKHSGQCLKPCKDAGMRFGKCMNGKCDCTPK

SEQ ID NO: 72 - protein, 32 aa, Artificial Sequence
  Description of Artificial Sequence: Synthetic polypeptide
GIVCKVCKIICGMQGKKVNICKAPIKCKCKKG

SEQ ID NO: 73 - protein, 37 aa, Artificial Sequence
  Description of Artificial Sequence: Synthetic polypeptide
DCVRFWGKCSQTSDCCPHLACKSKWPRNICVWDGSVG

SEQ ID NO: 74 - protein, 33 aa, Artificial Sequence
  Description of Artificial Sequence: Synthetic polypeptide
AVCVYRTCDKDCKRRGYRSGKCINNACKCYPYG

SEQ ID NO: 75 - protein, 33 aa, Artificial Sequence
  Description of Artificial Sequence: Synthetic polypeptide
GCFGYKCDYYKGCCSGYVCSPTWKWCVRPGPGR

SEQ ID NO: 76 - protein, 38 aa, Artificial Sequence
  Description of Artificial Sequence: Synthetic polypeptide
QVQTNVKCQGGSCASVCRREIGVAAGKCINGKCVCYRN

SEQ ID NO: 77 - protein, 33 aa, Artificial Sequence
  Description of Artificial Sequence: Synthetic polypeptide
GDCLPHLKRCKENNDCCSKKCKRRGANPEKRCR

SEQ ID NO: 78 - protein, 35 aa, Artificial Sequence
  Description of Artificial Sequence: Synthetic polypeptide
NFKVEGACSKPCRKYCIDKGARNGKCINGRCHCYY

SEQ ID NO: 79 - protein, 38 aa, Artificial Sequence
  Description of Artificial Sequence: Synthetic polypeptide
QKILSNRCNNSSECIPHCIRIFGTRAAKCINRKCYCYP

SEQ ID NO: 80 - protein, 42 aa, Artificial Sequence
  Description of Artificial Sequence: Synthetic polypeptide
DRDSCIDKSRCSKYGYYQECQDCCKKAGHNGGTCMFFKCKCA

SEQ ID NO: 81 - protein, 32 aa, Artificial Sequence
  Description of Artificial Sequence: Synthetic polypeptide
AVCNLKRCQLSCRSLGLLGKCIGDKCECVKHG

SEQ ID NO: 82 - protein, 37 aa, Artificial Sequence
  Description of Artificial Sequence: Synthetic polypeptide
QFCGTNGKPCVNGQCCGALRCVVTYHYADGVCLKMNP

SEQ ID NO: 83 - protein, 36 aa, Artificial Sequence
  Description of Artificial Sequence: Synthetic polypeptide
RPTDIKCSASYQCFPVCKSRFGKTNGRCVNGLCDCF

SEQ ID NO: 84 - protein, 35 aa, Artificial Sequence
  Description of Artificial Sequence: Synthetic polypeptide
NCAGYMRECKEKLCCSGYVCSSRWKWCVLPAPWRR

SEQ ID NO: 85 - protein, 37 aa, Artificial Sequence
  Description of Artificial Sequence: Synthetic polypeptide
QFTDVKCTGSKQCWPVCKQMFGKPNGKCMNGKCRCYS

SEQ ID NO: 86 - protein, 38 aa, Artificial Sequence
  Description of Artificial Sequence: Synthetic polypeptide
QIDTNVKCSGSSKCVKICIDRYNTRGAKCINGRCTCYP

SEQ ID NO: 87 - protein, 38 aa, Artificial Sequence
  Description of Artificial Sequence: Synthetic polypeptide
AEIIRCSGTRECYAPCQKLTGCLNAKCMNKACKCYGCV

SEQ ID NO: 88 - protein, 36 aa, Artificial Sequence
  Description of Artificial Sequence: Synthetic polypeptide
SDYCSNDFCFFSCRRDRCARGDCENGKCVCKNCHLN

SEQ ID NO: 89 - protein, 39 aa, Artificial Sequence
  Description of Artificial Sequence: Synthetic polypeptide
CIGEGVPCDENDPRCCFGLVCLKPTLHGIWYKSYYCYKK

SEQ ID NO: 90 - protein, 31 aa, Artificial Sequence
  Description of Artificial Sequence: Synthetic polypeptide
SCAKPGEMCMRIKCCDGQCGCNRGTGRCFCK

SEQ ID NO: 91 - protein, 35 aa, Artificial Sequence
  Description of Artificial Sequence: Synthetic polypeptide
ACLAEYQKCEGSTVPCCPGLSCSAGRFRKTKLCTK

SEQ ID NO: 92 - protein, 35 aa, Artificial Sequence
  Description of Artificial Sequence: Synthetic polypeptide
VVIGQRCYRSPDCYSACKKLVGKATGKCTNGRCDC

SEQ ID NO: 93 - protein, 32 aa, Artificial Sequence
  Description of Artificial Sequence: Synthetic polypeptide
ACQFWSCNSSCISRGYRQGYCWGIQYKYCQCQ

SEQ ID NO: 94 - protein, 36 aa, Artificial Sequence
  Description of Artificial Sequence: Synthetic polypeptide
RCPPCFTTNPNMEADCRKCCGGRGYCASYQCICPGG

SEQ ID NO: 95 - protein, 37 aa, Artificial Sequence
  Description of Artificial Sequence: Synthetic polypeptide
VFINVKCRGSPECLPKCKEAIGKSAGKCMNGKCKCYP

SEQ ID NO: 96 - protein, 38 aa, Artificial Sequence
  Description of Artificial Sequence: Synthetic polypeptide
QVSTNKKCSNTSQCYKTCEKVVGVAAGKCMNGKCICYP

SEQ ID NO: 97 - protein, 36 aa, Artificial Sequence
  Description of Artificial Sequence: Synthetic polypeptide
ECLEIFKACNPSNDQCCKSSKLVCSRKTRWCKYQIG

SEQ ID NO: 98 - protein, 54 aa, Artificial Sequence
  Description of Artificial Sequence: Synthetic polypeptide
QDKCKKVYENYPVSKCQLANQCNYDCKLDKHARSGECFYDEKRNLQCICDYCEY

SEQ ID NO: 99 - protein, 22 aa, Artificial Sequence
  Description of Artificial Sequence: Synthetic peptide
GHACYRNCWREGNDEETCKERC

SEQ ID NO: 100 - protein, 36 aa, Artificial Sequence
  Description of Artificial Sequence: Synthetic polypeptide
MCMPCFTTDTQMQERCDRCCGGGGRGRCWGPQCLCI

SEQ ID NO: 101 - protein, 36 aa, Artificial Sequence
  Description of Artificial Sequence: Synthetic polypeptide
MCMPCFTTEQRMAIICDDCCGGFGRGRCYGPQCLCR

SEQ ID NO: 102 - protein, 36 aa, Artificial Sequence
  Description of Artificial Sequence: Synthetic polypeptide
ICIPCFTTDHQIARRCDDCCGGRGRGRCYGPQCICR

SEQ ID NO: 103 - protein, 42 aa, Artificial Sequence
  Description of Artificial Sequence: Synthetic polypeptide
RCQLQGFNCVVRSYGLPTIPCCRGLTCRSYFPGSTYGRCQRY

SEQ ID NO: 104 - protein, 38 aa, Artificial Sequence
  Description of Artificial Sequence: Synthetic polypeptide
SFGLCRLRRGFCARGRCRFPSIPIGRCSRFVQCCRRVW

SEQ ID NO: 105 - protein, 33 aa, Artificial Sequence
  Description of Artificial Sequence: Synthetic polypeptide
SCEPGTTFRDRCNTCRCGSDGRSAACTLRACPQ

SEQ ID NO: 106 - protein, 36 aa, Artificial Sequence
  Description of Artificial Sequence: Synthetic polypeptide
SCTPGTTFRDRCNTCRCSSNGRSAACTLRACPPGSY

SEQ ID NO: 107 - protein, 36 aa, Artificial Sequence
  Description of Artificial Sequence: Synthetic polypeptide
SCTPGTTFRNRCNTCRCGSNGRSASCTLMACPPGSY

SEQ ID NO: 108 - protein, 36 aa, Artificial Sequence
  Description of Artificial Sequence: Synthetic polypeptide
SCTPGATFRNRCNTCRCGSNGRSASCTLMACPPGSY

SEQ ID NO: 109 - protein, 31 aa, Artificial Sequence
  Description of Artificial Sequence: Synthetic polypeptide
SCQPGTTYQRGCNTCRCLEDGQTEACTLRLC

SEQ ID NO: 110 - protein, 36 aa, Artificial Sequence
  Description of Artificial Sequence: Synthetic polypeptide
SCTPGATYREGCNICRCRSDGRSGACTRRICPVDSN

SEQ ID NO: 111 - protein, 32 aa, Artificial Sequence
  Description of Artificial Sequence: Synthetic polypeptide
SCQPGTTFRRDCNTCVCNRDGTNAACTLRACL

SEQ ID NO: 112 - protein, 44 aa, Artificial Sequence
  Description of Artificial Sequence: Synthetic polypeptide
YSRCQLQGFNCVVRSYGLPTIPCCRGLTCRSYFPGSTYGRCQRY

SEQ ID NO: 113 - protein, 34 aa, Artificial Sequence
  Description of Artificial Sequence: Synthetic polypeptide
SCARPRENCNRMNILCCRGECVCPTFGDCFCYGD

SEQ ID NO: 114 - protein, 37 aa, Artificial Sequence
  Description of Artificial Sequence: Synthetic polypeptide
GVPINVRCRGSRDCLDPCRRAGMRFGRCINSRCHCTP

SEQ ID NO: 115 - protein, 35 aa, Artificial Sequence
  Description of Artificial Sequence: Synthetic polypeptide
SERDCIRHLQRCRENRDCCSRRCSRRGTNPERRCR

SEQ ID NO: 116 - protein, 37 aa, Artificial Sequence
  Description of Artificial Sequence: Synthetic polypeptide
VRIPVSCRHSGQCLRPCRDAGMRFGRCMNGRCDCTPR

SEQ ID NO: 117 - protein, 38 aa, Artificial Sequence
  Description of Artificial Sequence: Synthetic polypeptide
QVQTNVRCQGGSCASVCRREIGVAAGRCINGRCVCYRN

SEQ ID NO: 118 - protein, 33 aa, Artificial Sequence
  Description of Artificial Sequence: Synthetic polypeptide
GDCLPHLRRCRENNDCCSRRCRRRGANPERRCR

SEQ ID NO: 119 - protein, 39 aa, Artificial Sequence
  Description of Artificial Sequence: Synthetic polypeptide
GSGVPINVRSRGSRDSLDPSRRAGMRFGRSINSRSHSTP

SEQ ID NO: 120 - skipped

SEQ ID NO: 121 - protein, 17 aa, Artificial Sequence
  Description of Artificial Sequence: Synthetic peptide
KKEEEKKEEEKKEEEKK

SEQ ID NO: 122 - protein, 34 aa, Artificial Sequence
  Description of Artificial Sequence: Synthetic polypeptide
GGDCLPHLRRCRADNDCCGRRCRRRGTNAERRCR

SEQ ID NO: 123 - protein, 46 aa, Artificial Sequence
  Description of Artificial Sequence: Synthetic polypeptide
GGDCKYKFENWGACDGGTGTKVRQGTLKKARYNAQCQETIRVTKPC

SEQ ID NO: 124 - protein, 63 aa, Artificial Sequence
  Description of Artificial Sequence: Synthetic polypeptide
GGDCKYKFENWGACDGGTGTKVRQGTLKKARYNAQCQETIRVTKPCTPKTKAKAKAKKGKGKD

SEQ ID NO: 125 - protein, 36 aa, Artificial Sequence
  Description of Artificial Sequence: Synthetic polypeptide
GGSCEPGRTFRDRCNTCRCGADGRSAACTLRACPNQ

SEQ ID NO: 126 - protein, 39 aa, Artificial Sequence
  Description of Artificial Sequence: Synthetic polypeptide
GGQFTNVSCTTSRECWSVCQRLHNTSRGRCMNRRCRCYS

SEQ ID NO: 127 - protein, 38 aa, Artificial Sequence
  Description of Artificial Sequence: Synthetic polypeptide
GGMCMPCFTTDHQMARRCDDCCGGRGRGRCYGPQCLCR

SEQ ID NO: 128 - protein, 39 aa, Artificial Sequence
  Description of Artificial Sequence: Synthetic polypeptide
GGISIGIKCSPSIDLCEGQCRIRKYFTGYCSGDTCHCSG

SEQ ID NO: 129 - protein, 39 aa, Artificial Sequence
  Description of Artificial Sequence: Synthetic polypeptide
GGEVIRCSGSKQCYGPCKQQTGCTNSKCMNKVCKCYGCG

SEQ ID NO: 130 - protein, 37 aa, Artificial Sequence
  Description of Artificial Sequence: Synthetic polypeptide
GGSEKDCIKHLQRCRENKDCCSKKCSRRGTNPEKRCR

SEQ ID NO: 131 - protein, 36 aa, Artificial Sequence
  Description of Artificial Sequence: Synthetic polypeptide
GGSCAKPRENCNRMNILCCRGECVCPTFGDCFCYGD

SEQ ID NO: 132 - protein, 39 aa, Artificial Sequence
  Description of Artificial Sequence: Synthetic polypeptide
GGGVPINVKCRGSRDCLDPCKKAGMRFGKCINSKCHCTP

SEQ ID NO: 133 - protein, 39 aa, Artificial Sequence
  Description of Artificial Sequence: Synthetic polypeptide
GGVRIPVSCKHSGQCLKPCKDAGMRFGKCMNGKCDCTPK

SEQ ID NO: 134 - protein, 34 aa, Artificial Sequence
  Description of Artificial Sequence: Synthetic polypeptide
GGGIVCKVCKIICGMQGKKVNICKAPIKCKCKKG

SEQ ID NO: 135 - protein, 39 aa, Artificial Sequence
  Description of Artificial Sequence: Synthetic polypeptide
GGDCVRFWGKCSQTSDCCPHLACKSKWPRNICVWDGSVG

SEQ ID NO: 136 - protein, 35 aa, Artificial Sequence
  Description of Artificial Sequence: Synthetic polypeptide
GGAVCVYRTCDKDCKRRGYRSGKCINNACKCYPYG

SEQ ID NO: 137 - protein, 35 aa, Artificial Sequence
  Description of Artificial Sequence: Synthetic polypeptide
GGGCFGYKCDYYKGCCSGYVCSPTWKWCVRPGPGR

SEQ ID NO: 138 - protein, 40 aa, Artificial Sequence
  Description of Artificial Sequence: Synthetic polypeptide
GGQVQTNVKCQGGSCASVCRREIGVAAGKCINGKCVCYRN

SEQ ID NO: 139 - protein, 35 aa, Artificial Sequence
  Description of Artificial Sequence: Synthetic polypeptide
GGGDCLPHLKRCKENNDCCSKKCKRRGANPEKRCR

SEQ ID NO: 140 - protein, 37 aa, Artificial Sequence
  Description of Artificial Sequence: Synthetic polypeptide
GGNFKVEGACSKPCRKYCIDKGARNGKCINGRCHCYY

SEQ ID NO: 141 - protein, 40 aa, Artificial Sequence
  Description of Artificial Sequence: Synthetic polypeptide
GGQKILSNRCNNSSECIPHCIRIFGTRAAKCINRKCYCYP

SEQ ID NO: 142 - protein, 44 aa, Artificial Sequence
  Description of Artificial Sequence: Synthetic polypeptide
GGDRDSCIDKSRCSKYGYYQECQDCCKKAGHNGGTCMFFKCKCA

SEQ ID NO: 143 - protein, 34 aa, Artificial Sequence
  Description of Artificial Sequence: Synthetic polypeptide
GGAVCNLKRCQLSCRSLGLLGKCIGDKCECVKHG

SEQ ID NO: 144 - protein, 39 aa, Artificial Sequence
  Description of Artificial Sequence: Synthetic polypeptide
GGQFCGTNGKPCVNGQCCGALRCVVTYHYADGVCLKMNP

SEQ ID NO: 145 - protein, 38 aa, Artificial Sequence
  Description of Artificial Sequence: Synthetic polypeptide
GGRPTDIKCSASYQCFPVCKSRFGKTNGRCVNGLCDCF

SEQ ID NO: 146 - protein, 37 aa, Artificial Sequence
  Description of Artificial Sequence: Synthetic polypeptide
GGNCAGYMRECKEKLCCSGYVCSSRWKWCVLPAPWRR

SEQ ID NO: 147 - protein, 39 aa, Artificial Sequence
  Description of Artificial Sequence: Synthetic polypeptide
GGQFTDVKCTGSKQCWPVCKQMFGKPNGKCMNGKCRCYS

SEQ ID NO: 148 - protein, 40 aa, Artificial Sequence
  Description of Artificial Sequence: Synthetic polypeptide
GGQIDTNVKCSGSSKCVKICIDRYNTRGAKCINGRCTCYP

SEQ ID NO: 149 - protein, 40 aa, Artificial Sequence
  Description of Artificial Sequence: Synthetic polypeptide
GGAEIIRCSGTRECYAPCQKLTGCLNAKCMNKACKCYGCV

SEQ ID NO: 150 - protein, 38 aa, Artificial Sequence
  Description of Artificial Sequence: Synthetic polypeptide
GGSDYCSNDFCFFSCRRDRCARGDCENGKCVCKNCHLN

SEQ ID NO: 151 - protein, 41 aa, Artificial Sequence
  Description of Artificial Sequence: Synthetic polypeptide
GGCIGEGVPCDENDPRCCFGLVCLKPTLHGIWYKSYYCYKK

SEQ ID NO: 152 - protein, 33 aa, Artificial Sequence
  Description of Artificial Sequence: Synthetic polypeptide
GGSCAKPGEMCMRIKCCDGQCGCNRGTGRCFCK

SEQ ID NO: 153 - protein, 37 aa, Artificial Sequence
  Description of Artificial Sequence: Synthetic polypeptide
GGACLAEYQKCEGSTVPCCPGLSCSAGRFRKTKLCTK

SEQ ID NO: 154 - protein, 37 aa, Artificial Sequence
  Description of Artificial Sequence: Synthetic polypeptide
GGVVIGQRCYRSPDCYSACKKLVGKATGKCTNGRCDC

SEQ ID NO: 155 - protein, 34 aa, Artificial Sequence
  Description of Artificial Sequence: Synthetic polypeptide
GGACQFWSCNSSCISRGYRQGYCWGIQYKYCQCQ

SEQ ID NO: 156 - protein, 38 aa, Artificial Sequence
  Description of Artificial Sequence: Synthetic polypeptide
GGRCPPCFTTNPNMEADCRKCCGGRGYCASYQCICPGG

SEQ ID NO: 157 - protein, 39 aa, Artificial Sequence
  Description of Artificial Sequence: Synthetic polypeptide
GGVFINVKCRGSPECLPKCKEAIGKSAGKCMNGKCKCYP

SEQ ID NO: 158 - protein, 40 aa, Artificial Sequence
  Description of Artificial Sequence: Synthetic polypeptide
GGQVSTNKKCSNTSQCYKTCEKVVGVAAGKCMNGKCICYP

SEQ ID NO: 159 - protein, 38 aa, Artificial Sequence
  Description of Artificial Sequence: Synthetic polypeptide
GGECLEIFKACNPSNDQCCKSSKLVCSRKTRWCKYQIG

SEQ ID NO: 160 - protein, 56 aa, Artificial Sequence
  Description of Artificial Sequence: Synthetic polypeptide
GGQDKCKKVYENYPVSKCQLANQCNYDCKLDKHARSGECFYDEKRNLQCICDYCEY

SEQ ID NO: 161 - protein, 24 aa, Artificial Sequence
  Description of Artificial Sequence: Synthetic peptide
GGGHACYRNCWREGNDEETCKERC

SEQ ID NO: 162 - protein, 38 aa, Artificial Sequence
  Description of Artificial Sequence: Synthetic polypeptide
GGMCMPCFTTDTQMQERCDRCCGGGGRGRCWGPQCLCI

SEQ ID NO: 163 - protein, 38 aa, Artificial Sequence
  Description of Artificial Sequence: Synthetic polypeptide
GGMCMPCFTTEQRMAIICDDCCGGFGRGRCYGPQCLCR

SEQ ID NO: 164 - protein, 38 aa, Artificial Sequence
  Description of Artificial Sequence: Synthetic polypeptide
GGICIPCFTTDHQIARRCDDCCGGRGRGRCYGPQCICR

SEQ ID NO: 165 - protein, 44 aa, Artificial Sequence
  Description of Artificial Sequence: Synthetic polypeptide
GGRCQLQGFNCVVRSYGLPTIPCCRGLTCRSYFPGSTYGRCQRY

SEQ ID NO: 166 - protein, 40 aa, Artificial Sequence
  Description of Artificial Sequence: Synthetic polypeptide
GGSFGLCRLRRGFCARGRCRFPSIPIGRCSRFVQCCRRVW

SEQ ID NO: 167 - protein, 35 aa, Artificial Sequence
  Description of Artificial Sequence: Synthetic polypeptide
GGSCEPGTTFRDRCNTCRCGSDGRSAACTLRACPQ

SEQ ID NO: 168 - protein, 38 aa, Artificial Sequence
  Description of Artificial Sequence: Synthetic polypeptide
GGSCTPGTTFRDRCNTCRCSSNGRSAACTLRACPPGSY

SEQ ID NO: 169 - protein, 38 aa, Artificial Sequence
  Description of Artificial Sequence: Synthetic polypeptide
GGSCTPGTTFRNRCNTCRCGSNGRSASCTLMACPPGSY

SEQ ID NO: 170 - protein, 38 aa, Artificial Sequence
  Description of Artificial Sequence: Synthetic polypeptide
GGSCTPGATFRNRCNTCRCGSNGRSASCTLMACPPGSY

SEQ ID NO: 171 - protein, 33 aa, Artificial Sequence
  Description of Artificial Sequence: Synthetic polypeptide
GGSCQPGTTYQRGCNTCRCLEDGQTEACTLRLC

SEQ ID NO: 172 - protein, 38 aa, Artificial Sequence
  Description of Artificial Sequence: Synthetic polypeptide
GGSCTPGATYREGCNICRCRSDGRSGACTRRICPVDSN

SEQ ID NO: 173 - protein, 34 aa, Artificial Sequence
  Description of Artificial Sequence: Synthetic polypeptide
GGSCQPGTTFRRDCNTCVCNRDGTNAACTLRACL

SEQ ID NO: 174 - protein, 46 aa, Artificial Sequence
  Description of Artificial Sequence: Synthetic polypeptide
GSYSRCQLQGFNCVVRSYGLPTIPCCRGLTCRSYFPGSTYGRCQRY

SEQ ID NO: 175 - protein, 36 aa, Artificial Sequence
  Description of Artificial Sequence: Synthetic polypeptide
GGSCARPRENCNRMNILCCRGECVCPTFGDCFCYGD

SEQ ID NO: 176 - protein, 39 aa, Artificial Sequence
  Description of Artificial Sequence: Synthetic polypeptide
GGGVPINVRCRGSRDCLDPCRRAGMRFGRCINSRCHCTP

SEQ ID NO: 177 - protein, 37 aa, Artificial Sequence
  Description of Artificial Sequence: Synthetic polypeptide
GGSERDCIRHLQRCRENRDCCSRRCSRRGTNPERRCR

SEQ ID NO: 178 - protein, 39 aa, Artificial Sequence
  Description of Artificial Sequence: Synthetic polypeptide
GGVRIPVSCRHSGQCLRPCRDAGMRFGRCMNGRCDCTPR

SEQ ID NO: 179 - protein, 40 aa, Artificial Sequence
  Description of Artificial Sequence: Synthetic polypeptide
GGQVQTNVRCQGGSCASVCRREIGVAAGRCINGRCVCYRN

SEQ ID NO: 180 - protein, 35 aa, Artificial Sequence
  Description of Artificial Sequence: Synthetic polypeptide
GGGDCLPHLRRCRENNDCCSRRCRRRGANPERRCR